SEQUENCE LISTING (97 sequences)

SEQ ID NO: 1 - DNA, 20 bp, synthetic construct
  source 1..20: mol_type = other DNA
cgcatacagtcttrcaggct

SEQ ID NO: 2 - DNA, 20 bp, synthetic construct
  source 1..20: mol_type = other DNA
ggagttgtgatgggagtgat

SEQ ID NO: 3 - DNA, 22 bp, synthetic construct
  source 1..22: mol_type = other DNA
tccattggtcttctgtcacccg

SEQ ID NO: 4 - DNA, 21 bp, synthetic construct
  source 1..21: mol_type = other DNA
ctctacggaccaaacttcatc

SEQ ID NO: 5 - DNA, 22 bp, synthetic construct
  source 1..22: mol_type = other DNA
ggctgtgaagtcgtcagaacac

SEQ ID NO: 6 - DNA, 20 bp, synthetic construct
  source 1..20: mol_type = other DNA
tgaacctcagctacaaacag

SEQ ID NO: 7 - DNA, 20 bp, synthetic construct
  source 1..20: mol_type = other DNA
cctcacttcactgcactgta

SEQ ID NO: 8 - DNA, 24 bp, synthetic construct
  source 1..24: mol_type = other DNA
ttcacatgtcccagcactaccaga

SEQ ID NO: 9 - DNA, 23 bp, synthetic construct
  source 1..23: mol_type = other DNA
ttcactacagtcgctacatcacc

SEQ ID NO: 10 - DNA, 20 bp, synthetic construct
  source 1..20: mol_type = other DNA
acatctcgcccttcagccac

SEQ ID NO: 11 - DNA, 20 bp, synthetic construct
  source 1..20: mol_type = other DNA
aggatgtggatgctgcctac

SEQ ID NO: 12 - DNA, 22 bp, synthetic construct
  source 1..22: mol_type = other DNA
caggtggtggagaaagttgaga

SEQ ID NO: 13 - DNA, 30 bp, synthetic construct
  source 1..30: mol_type = other DNA
acaggatccatgttgtggaaaataaccgat

SEQ ID NO: 14 - DNA, 22 bp, synthetic construct
  source 1..22: mol_type = other DNA
agaaacgaagatccccagatga

SEQ ID NO: 15 - DNA, 19 bp, synthetic construct
  source 1..19: mol_type = other DNA
accctggctgtggagaagg

SEQ ID NO: 16 - DNA, 22 bp, synthetic construct
  source 1..22: mol_type = other DNA
gttaatgacatcaaaggcaccc

SEQ ID NO: 17 - DNA, 20 bp, synthetic construct
  source 1..20: mol_type = other DNA
gctgaccttcacactcccca

SEQ ID NO: 18 - DNA, 20 bp, synthetic construct
  source 1..20: mol_type = other DNA
cagatacctggagaacggga

SEQ ID NO: 19 - DNA, 22 bp, synthetic construct
  source 1..22: mol_type = other DNA
tggcacccatttacacctacac

SEQ ID NO: 20 - DNA, 23 bp, synthetic construct
  source 1..23: mol_type = other DNA
ctcaacacaataaaggagctggg

SEQ ID NO: 21 - DNA, 21 bp, synthetic construct
  source 1..21: mol_type = other DNA
tttctcaggcccaattttggt

SEQ ID NO: 22 - DNA, 21 bp, synthetic construct
  source 1..21: mol_type = other DNA
ggagcacacgctagttcagaa

SEQ ID NO: 23 - DNA, 20 bp, synthetic construct
  source 1..20: mol_type = other DNA
tctgggcatcccaagttgac

SEQ ID NO: 24 - DNA, 23 bp, synthetic construct
  source 1..23: mol_type = other DNA
taaccctggctttggaatcaaaa

SEQ ID NO: 25 - DNA, 20 bp, synthetic construct
  source 1..20: mol_type = other DNA
tggtcatcgcccaagcactg

SEQ ID NO: 26 - DNA, 22 bp, synthetic construct
  source 1..22: mol_type = other DNA
gagcaacttctttcaaccacag

SEQ ID NO: 27 - DNA, 22 bp, synthetic construct
  source 1..22: mol_type = other DNA
agcattacaacaacctgctacg

SEQ ID NO: 28 - DNA, 25 bp, synthetic construct
  source 1..25: mol_type = other DNA
cttgaaggacagacatgactttgga

SEQ ID NO: 29 - DNA, 20 bp, synthetic construct
  source 1..20: mol_type = other DNA
aaactcatgagcagtctgca

SEQ ID NO: 30 - DNA, 20 bp, synthetic construct
  source 1..20: mol_type = other DNA
tggcttttcagctctgcatc

SEQ ID NO: 31 - DNA, 20 bp, synthetic construct
  source 1..20: mol_type = other DNA
cgccttggaagagtcactca

SEQ ID NO: 32 - DNA, 20 bp, synthetic construct
  source 1..20: mol_type = other DNA
agttccgggcctgtatccag

SEQ ID NO: 33 - DNA, 22 bp, synthetic construct
  source 1..22: mol_type = other DNA
tcgcttctgctgaaggactgca

SEQ ID NO: 34 - DNA, 18 bp, synthetic construct
  source 1..18: mol_type = other DNA
atgcggccgagtgtctgg

SEQ ID NO: 35 - DNA, 20 bp, synthetic construct
  source 1..20: mol_type = other DNA
gtcaggggtggttattgcat

SEQ ID NO: 36 - DNA, 17 bp, synthetic construct
  source 1..17: mol_type = other DNA
tccagtcacggtcagca

SEQ ID NO: 37 - DNA, 22 bp, synthetic construct
  source 1..22: mol_type = other DNA
ctcttctgcctgctgcactttg

SEQ ID NO: 38 - DNA, 20 bp, synthetic construct
  source 1..20: mol_type = other DNA
cgaggatggccgtcatggcg

SEQ ID NO: 39 - DNA, 20 bp, synthetic construct
  source 1..20: mol_type = other DNA
cagttcgtgaggttcgacag

SEQ ID NO: 40 - DNA, 23 bp, synthetic construct
  source 1..23: mol_type = other DNA
ctggacatagtcatagtgctgga

SEQ ID NO: 41 - DNA, 21 bp, synthetic construct
  source 1..21: mol_type = other DNA
gtcgggggcttcaacttagac

SEQ ID NO: 42 - DNA, 20 bp, synthetic construct
  source 1..20: mol_type = other DNA
cacatctcctccctgagcac

SEQ ID NO: 43 - DNA, 23 bp, synthetic construct
  source 1..23: mol_type = other DNA
ttcactacagtcgctacatcacc

SEQ ID NO: 44 - DNA, 21 bp, synthetic construct
  source 1..21: mol_type = other DNA
tgccctcacggtaatgttgga

SEQ ID NO: 45 - DNA, 20 bp, synthetic construct
  source 1..20: mol_type = other DNA
caaattccatggcaccgtca

SEQ ID NO: 46 - DNA, 19 bp, synthetic construct
  source 1..19: mol_type = other DNA
gtagcagcattgtacaggg

SEQ ID NO: 47 - DNA, 22 bp, synthetic construct
  source 1..22: mol_type = other DNA
gtttgggaaagtgcttcctttt

SEQ ID NO: 48 - DNA, 22 bp, synthetic construct
  source 1..22: mol_type = other DNA
gtttggatcgtgcatcctttta

SEQ ID NO: 49 - DNA, 19 bp, synthetic construct
  source 1..19: mol_type = other DNA
gtgcctctagatggaagca

SEQ ID NO: 50 - DNA, 19 bp, synthetic construct
  source 1..19: mol_type = other DNA
gttgaaggcgcttcccttt

SEQ ID NO: 51 - DNA, 24 bp, synthetic construct
  source 1..24: mol_type = other DNA
gtgtgatgttgawatgacatggtc

SEQ ID NO: 52 - DNA, 20 bp, synthetic construct
  source 1..20: mol_type = other DNA
gatggaggcataaggatttt

SEQ ID NO: 53 - DNA, 22 bp, synthetic construct
  source 1..22: mol_type = other DNA
agaccatccacctccacttctc

SEQ ID NO: 54 - DNA, 22 bp, synthetic construct
  source 1..22: mol_type = other DNA
ccactattccttggctagagta

SEQ ID NO: 55 - DNA, 21 bp, synthetic construct
  source 1..21: mol_type = other DNA
acctgctctcggagccgttca

SEQ ID NO: 56 - DNA, 20 bp, synthetic construct
  source 1..20: mol_type = other DNA
tggtggtaggaagagtaaag

SEQ ID NO: 57 - DNA, 20 bp, synthetic construct
  source 1..20: mol_type = other DNA
caggttttctttccctagct

SEQ ID NO: 58 - DNA, 26 bp, synthetic construct
  source 1..26: mol_type = other DNA
tcacatgtgtgagaggggcagtgtgc

SEQ ID NO: 59 - DNA, 22 bp, synthetic construct
  source 1..22: mol_type = other DNA
ttgattttcctctcctttgctc

SEQ ID NO: 60 - DNA, 21 bp, synthetic construct
  source 1..21: mol_type = other DNA
catggcggtgaccatgctgga

SEQ ID NO: 61 - DNA, 20 bp, synthetic construct
  source 1..20: mol_type = other DNA
caccacagatgtgtcggaga

SEQ ID NO: 62 - DNA, 22 bp, synthetic construct
  source 1..22: mol_type = other DNA
gtgcttgagcttgtggatgaag

SEQ ID NO: 63 - DNA, 30 bp, synthetic construct
  source 1..30: mol_type = other DNA
atactcgagtttcctgtgtttctccatttt

SEQ ID NO: 64 - DNA, 22 bp, synthetic construct
  source 1..22: mol_type = other DNA
ctgttgctgttgcctgtacgtt

SEQ ID NO: 65 - DNA, 18 bp, synthetic construct
  source 1..18: mol_type = other DNA
atggactcgaagcgcaca

SEQ ID NO: 66 - DNA, 21 bp, synthetic construct
  source 1..21: mol_type = other DNA
ccccatctcaacaggaaaacc

SEQ ID NO: 67 - DNA, 21 bp, synthetic construct
  source 1..21: mol_type = other DNA
caaaggtgaagtcctgctccc

SEQ ID NO: 68 - DNA, 20 bp, synthetic construct
  source 1..20: mol_type = other DNA
cagtatgatctccgcagggt

SEQ ID NO: 69 - DNA, 22 bp, synthetic construct
  source 1..22: mol_type = other DNA
atgtcaggagaggccccataga

SEQ ID NO: 70 - DNA, 23 bp, synthetic construct
  source 1..23: mol_type = other DNA
aaaggggatacaggtttttccac

SEQ ID NO: 71 - DNA, 21 bp, synthetic construct
  source 1..21: mol_type = other DNA
gctgtcggaggtatatggtgg

SEQ ID NO: 72 - DNA, 21 bp, synthetic construct
  source 1..21: mol_type = other DNA
ggtctctattgaggggaaggt

SEQ ID NO: 73 - DNA, 21 bp, synthetic construct
  source 1..21: mol_type = other DNA
tccgccttttgatctccgatt

SEQ ID NO: 74 - DNA, 23 bp, synthetic construct
  source 1..23: mol_type = other DNA
tgggtacaaggtaaaatggcttt

SEQ ID NO: 75 - DNA, 20 bp, synthetic construct
  source 1..20: mol_type = other DNA
ggcggtaaatcgtccatgct

SEQ ID NO: 76 - DNA, 21 bp, synthetic construct
  source 1..21: mol_type = other DNA
cacttccttctgccaaacttg

SEQ ID NO: 77 - DNA, 21 bp, synthetic construct
  source 1..21: mol_type = other DNA
gttggggtcagccatactcag

SEQ ID NO: 78 - DNA, 21 bp, synthetic construct
  source 1..21: mol_type = other DNA
ggatggtttcagccttttgga

SEQ ID NO: 79 - DNA, 22 bp, synthetic construct
  source 1..22: mol_type = other DNA
aggagatcttcagtttcggagg

SEQ ID NO: 80 - DNA, 21 bp, synthetic construct
  source 1..21: mol_type = other DNA
ccgctacatctgaatgacctg

SEQ ID NO: 81 - DNA, 20 bp, synthetic construct
  source 1..20: mol_type = other DNA
gaagcctcaggtcccaattc

SEQ ID NO: 82 - DNA, 20 bp, synthetic construct
  source 1..20: mol_type = other DNA
gagccggtacagccaatggt

SEQ ID NO: 83 - DNA, 22 bp, synthetic construct
  source 1..22: mol_type = other DNA
cctccagaaccttcagcgtcag

SEQ ID NO: 84 - DNA, 19 bp, synthetic construct
  source 1..19: mol_type = other DNA
gctccagcgagcggtagtg

SEQ ID NO: 85 - DNA, 20 bp, synthetic construct
  source 1..20: mol_type = other DNA
agtgaggaacaagccagagc

SEQ ID NO: 86 - DNA, 21 bp, synthetic construct
  source 1..21: mol_type = other DNA
cagcctcagagtgtttcttct

SEQ ID NO: 87 - DNA, 22 bp, synthetic construct
  source 1..22: mol_type = other DNA
atgggctacaggcttgtcactc

SEQ ID NO: 88 - DNA, 23 bp, synthetic construct
  source 1..23: mol_type = other DNA
cacattccgtgtctcctggtccc

SEQ ID NO: 89 - DNA, 19 bp, synthetic construct
  source 1..19: mol_type = other DNA
cagccgtacatgctctgga

SEQ ID NO: 90 - DNA, 21 bp, synthetic construct
  source 1..21: mol_type = other DNA
acctgtgtctgtttaggacca

SEQ ID NO: 91 - DNA, 20 bp, synthetic construct
  source 1..20: mol_type = other DNA
cctggctggctggtattagc

SEQ ID NO: 92 - DNA, 20 bp, synthetic construct
  source 1..20: mol_type = other DNA
tatcttgccacccatccttg

SEQ ID NO: 93 - DNA, 22 bp, synthetic construct
  source 1..22: mol_type = other DNA
ttgattttcctctcctttgctc

SEQ ID NO: 94 - DNA, 21 bp, synthetic construct
  source 1..21: mol_type = other DNA
tgatgctcaaaggcagggtag

SEQ ID NO: 95 - DNA, 20 bp, synthetic construct
  source 1..20: mol_type = other DNA
atcgccccacttgattttgg

SEQ ID NO: 96 - DNA, 20 bp, synthetic construct
  source 1..20: mol_type = other DNA
gtggcctggtcactcttaac

SEQ ID NO: 97 - DNA, 21 bp, synthetic construct
  source 1..21: mol_type = other DNA
caaataaaggcagctgctgtg

(19) United States
(12) Patent Application Publication
(10) Pub. No.: US 2023/0393122 A1
(43) Pub. Date: Dec. 7, 2023

(54) STEM CELL COMPOSITIONS FOR CULTURING CORONAVIRUSES AND METHODS OF MAKING AND USING THEREOF

(71) Applicant: Centre for Translational Stem Cell Biology Limited, Hong Kong (CN)

(72) Inventors: Degong Ruan, Hong Kong (CN); Pengtao Liu, Hong Kong (CN); Fang Liu, Hong Kong (CN)

(21) Appl. No.: 18/345,934

(22) Filed: Jun. 30, 2023

Related U.S. Application Data
(60) Provisional application No. 63/402,032, filed on Aug. 29, 2022.

(30) Foreign Application Priority Data
Jun. 6, 2022 (CN) 202210631030.7

(51) Int. Cl. G01N 33/50; C12N 7/00; C12Q 1/6851; C12Q 1/70

(52) U.S. Cl. CPC G01N 33/5091; C12N 7/00; C12Q 1/6851; C12Q 1/701; C12N 2770/20052; C12N 2770/20021; G01N 2333/165

(57) ABSTRACT

Disclosed are methods for culturing coronavirus particles in early syncytiotrophoblasts (eSTBs). The derived eSTBs are mononucleated or bi-nucleated cells with high ACE2 expression and are not multi-nucleated or mature cells. The methods can also include assessing the eSTBs for coronavirus susceptible markers. Also disclosed are compositions and methods (i) for inducing the differentiation of eSTBs and mature STBs from trophoblast stem cells (TSCs), (ii) for inducing the differentiation of TSCs from EPSCs, primed and naïve stem cells, pre-implantation embryos, placental stem cells, and iPSCs, and (ii) for producing TSCs by reprogramming non-trophoblast cells. The disclosed compositions and methods can be used for producing large quantities of coronavirus particles, including human, non-human, and variant coronavirus particles for virus production, the vaccine inductry, disease modeling studies, screening and evaluation of antiviral reagents, compound candidates, testing kits, and evaluation of clinical therapies.

### CROSS REFERENCE TO RELATED APPLICATIONS

This application claims benefit of and priority to U.S. Provisional Application No. 63/402,032, filed Aug. 29, 2022, and Chinese Application No. 202210631030.7, filed Jun. 6, 2022, which are specifically incorporated by reference herein in their entireties.

### REFERENCE TO THE SEQUENCE LISTING

The Sequence Listing submitted as a text file named “UHK_01180_US_ST26.xml” created on Jun. 19, 2023, and having a size of 86,578 bytes is hereby incorporated by reference pursuant to 37 C.F.R. § 1.834(c)(1).

### FIELD OF THE INVENTION

The disclosed invention is generally directed to compositions and methods for culturing coronavirus in early syncytiotrophoblasts.

### BACKGROUND OF THE INVENTION

Vero cells have long been used as the cell substrate of kinds of viruses, but it is an animal derived cell substrate. The viruses produced by the Vero cell line might induce different host cell reactions in humans. Several human cell lines for virus production, such as WI38 cell and PER C6 cell, are embryonic derived cell lines, which are quite limited for virus research and industry.

The human placenta protects the fetus using multiple cellular and molecular defense mechanisms at the maternal-fetal interface safeguarding against infection during pregnancy, but certain viruses still replicate in the placenta and infect the fetus (Pereira, 2018). SARS-CoV-2 infection of the human placenta and resultant damages, although relatively uncommon, have been reported that caused placental inflammation, trophoblast necrosis and chronic histiocytic intervillositis (placentitis), and miscarriage and stillbirth (Algarroba et al., 2020; Baud et al., 2020; Hosier et al., 2020; Male, 2022; Roberts et al., 2021; Schwartz et al., 2022; Valdespino-Vázquez et al., 2021; Vivanti et al., 2020). In rare cases, the exposure to maternal viremia followed by placental infection may lead to vertical transmission from mother to fetus (Allotey et al., 2022; Dong et al., 2020b; Vivanti et al., 2020). A recent report revealed that statistically better pregnancy outcomes are one health benefit of COVID-19 vaccination (Stock et al., 2022).

The human placenta consists of both maternal and fetal tissues (Burton and Fowden, 2015). The extraembryonic ectoderm in early post-implantation embryos generates the proliferative mononucleated trophoblast progenitors known as villous cytotrophoblasts (vCTBs), which can differentiate into invasive extravillous trophoblasts (EVTs) in the anchoring villi that grow out into the maternal decidua. vCTBs can also undergo active cellular fusion into non-proliferative multinucleated STBs that form a physical barrier to pathogens (Zeldovich et al., 2013).

SARS-CoV-2 infects cells via its spike (S) protein binding to the host entry receptor ACE2 (Letko et al., 2020; Zhou et al., 2020) and primed by the transmembrane serine protease 2 (TMPRSS2) (Hoffmann et al., 2020). Molecular assays and single-cell RNA sequencing (scRNAseq) studies have identified ACE2 and TMPRSS2 co-expression in a small number of first trimester STBs and second trimester EVTs (Ashary et al., 2020; Chen et al., 2020; Pique-Regi et al., 2020), which are gradually decreased during pregnancy (Ashary et al., 2020). Indeed, term placenta trophoblasts are not susceptible to SARS-CoV-2 and do not express ACE2 (Colson et al., 2021). Additionally, ACE2 shedding may help prevent SARS-CoV-2 infection from continuing to spread in the placenta (Taglauer et al., 2022). These molecular studies are in line with the overall low risk of COVID-19 to pregnant women. On the other hand, little is known about COVID-19's risk to early pregnancy since the impacts could be unnoticed and it is technically and ethically challenging to study normal trophoblasts of early pregnancy stages. Laboratory model organisms such as the mouse are used to study SARS-CoV-2 but they have substantial differences from humans in trophoblast biology and placenta development. Therefore, 2D and 3D cellular models of normal human trophoblasts are needed to decipher SARS-CoV-2 infection in trophoblasts and in early pregnancy.

Expanded potential stem cells (EPSCs) of several species derived from cleavage stage preimplantation embryos retain developmental potential to both extraembryonic as well as embryonic cell lineages (Gao et al., 2019; Yang et al., 2017a; Yang et al., 2017b; Zhao et al., 2021b). In particular, human EPSCs (hEPSCs) directly generated human TSCs (hTSCs) in vitro (Cinkornpumin et al., 2020; Gao et al., 2019). Standard human embryonic stem cells (hESCs) could also be induced to generate trophoblast-like cells (Amita et al., 2013; Horii et al., 2016; Xu et al., 2002) and to derive hTSC lines (Wei et al., 2021). hTSCs were recently established from human naïve ESCs (Castel et al., 2020; Cinkornpumin et al., 2020; Dong et al., 2020a; Guo et al., 2021; Jo et al., 2021), which may reflect the unique property of human naïve epiblast to regenerate trophoblast (Guo et al., 2021). Thus, there is an urgent need for improved cell models of normal human trophoblasts for coronavirus research. There is also an urgent need of improved cell models for producing coronavirus vaccines and for antiviral drug evaluation and discovery.

Therefore, it is an object of the invention to provide improved compositions and methods for isolating, propagating, and producing coronaviruses by hTSC and hSTB.

It is another object of the invention to provide improved compositions and methods for screening agents for the production of coronavirus vaccines and for antiviral drug evaluation and discovery.

### BRIEF SUMMARY OF THE INVENTION

A stem-cell based system for producing coronavirus particles has been established in which trophoblast stem cells (TSCs) are used to generate early syncytiotrophoblasts (eSTBs) for infection with coronavirus particles. TSCs can be derived from expanded potential stem cells (EPSCs), placental tissues, peri-implantation embryos, and induced pluripotent stem cells, or by reprogramming somatic cells such as fibroblasts and blood cells using genetic factors or small molecules. In some forms, the TSCs can be derived from naïve stem cells and/or primed stem cells. Typically, TSCs from any one of these sources are similar in gene expression and have differentiation potential to derive the disclosed eSTBs.

Disclosed are methods for culturing coronavirus particles in eSTBs, said method including incubating eSTBs in coronavirus medium containing coronavirus particles, whereby the coronavirus particles infect and replicate in the eSTBs.

In some forms, the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles, and/or variant particles thereof. Preferably the coronavirus particles are SARS-CoV-2 particles.

Typically, the derived eSTBs are isolated by selecting cells expressing eSTB-like morphology, cells expressing an eSTB-like molecular signature, or cells expressing both eSTB-like morphology and an eSTB-like molecular signature. In some forms, the eSTBs are mononucleated cells or bi-nucleated. In some forms, the eSTBs are not multi-nucleated or mature cells.

In some forms, the methods of culturing coronavirus particles in eSTBs also include assessing a portion of the isolated eSTBs for coronavirus susceptible markers. Typically, the coronavirus susceptible markers are increased ACE2 and/or increased TMPRSS2. In some forms, the assessment of the portion of the eSTBs for coronavirus susceptible markers is done via virus replication kinetic assays including but not limited to RT-qPCR, plaque assays, Trans-well invasion assays, and/or RNA sequencing.

In some forms, the methods of culturing coronavirus particles in eSTBs further include quantifying the coronavirus load in the coronavirus medium via for example, a plaque assay, RT-qPCR, and/or RNA sequencing. In some forms, when the coronavirus particles are SARS-CoV-2 particles, the coronavirus load is detected via RT-qPCR using the primers SEQ ID NO:1 and SEQ ID NO:51.

In some forms, the coronavirus medium contains basal medium, wherein basal medium is DMEM/F-12 or DMEM. In some forms, the coronavirus particles are isolated from a sample containing the coronavirus particles such as for example coronavirus infected VeroE6 cells.

Also disclosed are methods for screening an agent for an effect on early syncytiotrophoblasts (eSTBs). In some forms, the method includes contacting the eSTBs with the agent and determining the effect of the agent on survival, proliferation, differentiation, or morphologic, genetic, or functional parameters of the eSTBs.

In some forms, the effect of the agent is indicative of the agent being safe for treatment of a subject. In some forms, the effect of the agent is indicative of the agent being safe for treatment of a pregnant subject, a fetus infected with coronavirus, or both. In some forms, the agent is an antiviral agent, wherein the effect of the agent is indicative of the agent being safe for treatment of viral infections. In some forms, the agent is nucleic acids or analogs thereof, polypeptides or analogs thereof, antibodies, chemicals, small molecules, and/or any combination thereof. In some forms, the treated and untreated eSTBs are evaluated using PCR techniques, immunoassays, sequencing, biochemical assays, functional assays, cell viability assays, microscopy, or combinations thereof.

Also disclosed are kits for culturing the coronavirus particles in early syncytiotrophoblasts (eSTBs). In some forms, the kit includes a combination of two or more of: (i) the fourth culture medium for deriving the EPSCs described in A(ii) comprising one or more of a RAS-ERK inhibitor, a SRC kinase inhibitor, a GSK3 inhibitor, and/or a WNT inhibitor; (ii) the EPSC maintenance medium comprising one or more of a RAS-ERK inhibitor, a SRC kinase inhibitor, a GSK3 inhibitor and/or a WNT inhibitor; (iii) the dissociating reagent for producing single EPSCs comprising a trypsin replacement agent; (iv) the trophoblast stem cell medium comprising basal medium supplemented with one or more of a reducing agent, fetal bovine serum (FBS), an antibiotic, Bovine Serum Albumin (BSA), Epidermal Growth Factor (EGF), Glycogen synthase kinase 3 (GSK-3) inhibitor, an ALK-5 inhibitor, a ROCK inhibitor, a TGF-β inhibitor, and/or an HDAC inhibitor, wherein basal medium is DMEM/F-12 or DMEM; (v) STB medium comprising basal medium supplemented with one or more of a reducing agent, BSA, an antibiotic, a ROCK inhibitor, a cAMP inhibitor, KSR medium, and/or one or more differentiation agents; and (vi) coronavirus medium comprising basal medium, wherein basal medium is DMEM/F-12 or DMEM.

Also disclosed are compositions and methods for inducing the differentiation of TSCs to eSTBs and EVTs. Compositions and methods for inducing the differentiation of TSCs from EPSCs, placental tissues, peri-implantation embryos, primed stem cells, naïve stem cells, and induced pluripotent stem cells are also disclosed.

### BRIEF DESCRIPTION OF THE DRAWINGS

The accompanying drawings illustrate several forms of the disclosed method and compositions and together with the description, serve to explain the principles of the disclosed method and compositions.

FIG. 1 is a schematic diagram of the sequential generation of hTSCs, STBs, and EVTs from hEPSCs. Using the hTSC medium (hTSCM), EPSC-TSCs are directly established from hEPSCs. EPSC-TSCs are further differentiated into STBs or EVTs. The initially differentiated EPSC-TSCs toward STBs are mononucleated cells positive for early STB markers CD46 and SSEA4, while the late-stage differentiated cells are multinucleated expressing more mature STB marker CGB. EPSC-TSCs are also induced to generate EVTs that express HLA-G.

FIG. 2A is a bar graph of the RT-qPCR analysis of trophoblast genes ELF5, TFAP2C, TEAD4, TP63 and GATA3 in EPSC-TSCs (at passage 10) and BST-TSCs. Results are normalized to levels of GAPDH using the ΔCt method. FIG. 2B is a graph showing the low HLA-A and HLA-B levels in EPSC-TSCs and BST-TSC in RT-qPCR. Results are normalized to levels of GAPDH using the ΔCt method. FIG. 2C is a graph of the flow cytometry quantification of HLA-A, HLA-B, HLA-C on EPSC-TSCs and BST-TSCs. The antibody isotype is the control. FIG. 2D is a bar graph of the RT-qPCR results of CDX2 and putative AME genes MUC16 and GABRP and trophoblast genes ELF5 and GATA3 expression in EPSC-TSC and BST-TSC. Results are normalized to levels of GAPDH using the ΔCt method.

FIG. 3A is a bar graph of the RT-qPCR trophoblast-specific C19MC miRNAs (has-miR-517a, 517b, 525-3p and 526-3p) in hEPSCs, EPSC-TSCs and BST-TSC. Results are normalized to levels of miR-103a using the ΔCt method. FIG. 3B is a heat map of the results from the RNAseq analysis of human trophoblast and putative AME signature genes (Table 2) expressed in EPSC-TSCs and BST-TSC and CT-TSCs and five human placenta-derived CT-TSCs (CT27, 29, 30, BTS5 and BTS11-TSCs). Log₂ transformed cpm (counts per million) was used in generating the heatmap. Individual genes of the two gene sets are ordered by expression levels. All hTSCs express high levels of trophoblast genes but low levels of AME signature.

FIG. 4A are brightfield image quantification of EPSC-TSC differentiating toward STBs on day 6 (STB-D6). Scale bar:100 μm is a bar graph showing the ELISA (IU/L) detection of 3-hCG. Supernatant collected from STB cells generated from EPSC-TSCs on Day 2, 3 and 6 are used. Fresh STB medium was used as the blank control. FIGS. 4B-4E are line graphs of the RT-qPCR detection of decreased expression of TP63 (FIG. 4B) and increased STB markers, GCM1 (FIG. 4C) and CGB3 (FIG. 4D), and CGB5 (FIG. 4E), in EPSC-TSC differentiation toward STBs. STB-D2, -D4, -D6, and -D8 are days along the differentiation. Results are normalized to levels of GAPDH using the ΔCt method. FIGS. 4F and 4G are bright field images of EPSC-TSC differentiating towards EVT on day 8. FIG. 4H are immunofluorescence images of EPSC-TSC differentiation toward STBs on day 6 stained for STB markers GCM1 and CGB (upper panel); and GATA3 (lower panel). Arrows indicate the lack of GATA3 in a multinucleated CGB+ mature STB. Nuclei were counter stained with DAPI. FIG. 4I are micrographs of STBs generated from EPSC-TSCs have distinct morphologies and gene expression. They range from mono-, bi- or multinucleated STBs that express GCM1 and CGB. The multinucleated mature STBs exhibited dotted CGB staining in the cytoplasm. FIG. 4J is immunofluorescence images of EVTs generated from EPSC-TSCs stained for KRT7 and EVT specific marker HLA-G. Nuclei were counter stained with DAPI.

FIGS. 5A-5D are line graphs of the RT qPCR detection of decreased expression of GATA3 (FIG. 5A) and increased EVT markers ITGA1 (FIG. 5B), MMP2 (FIG. 5C) and HLA-G (FIG. 5D) in EPSC-TSC differentiation toward EVTs. EVT-D2, -D4, -D6 and -D8 are days along the differentiation. Results are normalized to levels of GAPDH using the ΔCt method. Three independent experiments were performed. Data are mean±SD. ***p<0.001 (two-tailed unpaired Student's t-test between EPSC-TSCs and EVT-D8).

FIGS. 6A-6C are graphs of the flow cytometry quantification of expression of HLA-A, HLA-B, HLA-C and EVT specific marker HLA-G on EVTs generated from EPSC-TSCs. EPSC-TSCs were used as the flow analysis control. Both TSCs and EVTs are negative for the HLA-A, HLA-B, and HLA-C antibody. FIGS. 6D-6G are graphs of the flow cytometry quantification of EVT markers ITGA5 (FIGS. 6D and 6E) and ITGA1 (FIGS. 6F and 6G) on EVTs differentiated from EPSC-TSCs (day 8).

FIGS. 7A and 7B are bar graphs showing the expression of HLA genes in the published human placenta-derived trophoblasts (Sheridan, M. A., 2021; FIG. 7A) and in EPSC-TSCs and their derivative trophoblasts (FIG. 7B) of the present study. FIG. 7C is a principal component analysis comparing the CT-derived trophoblasts from Sheridan et al., 2021 with the EPSC-derived trophoblasts of the current study and Gao et al., 2020). FIG. 7D is a heatmap showing RNAseq analysis of EPSC-TSCs and their derivatives STBs and EVTs, which expressed distinct sets of genes specific for human primary trophoblast progenitor, STB and EVT.

FIGS. 8A and 8B are uMAP representations containing scRNAseq data of cells from in vitro cultured peri-implantation embryo stages (top panel) and of cells from first and second trimester placenta (bottom panel). Cells are colored by developmental time points (ED6-14: Embryonic day 6-14. HE8W/HE24W: placenta trophoblasts at 8 weeks or 24 weeks of gestation, corresponding to first or second trimester) in (FIG. 8A), and trophoblast types at each stage in (FIG. 8B). PI: Peri-implantation; PI-TB: Peri-implantation trophoblast; PI-STB: Peri-implantation syncytiotrophoblast; PI-EVT: Peri-implantation extravillus trophoblast; T1-CTB, T1-STB and T1-EVT: first trimester cytotrophoblast, syncytiotrophoblasts and extravillus trophoblast; T2-EVT: second trimester extravillus trophoblast.

FIGS. 9A-9F are violin plots of trophoblast gene TP63 (FIG. 9A), TEAD4 (FIG. 9B), CGB5 (FIG. 9C), CSH2 (FIG. 9D), HLA-G (FIG. 9E) and MMP2 (FIG. 9F) (log-transformed TPM) in human peri-implantation embryos and placenta trophoblasts as shown in FIG. 8B. FIGS. 9G-9L are violin plots of expression of putative AME genes MUC16 (FIG. 9G), ITGB6 (FIG. 9H), GABRP (FIG. 9I), ISL1 (FIG. 9J), VTCN1 (FIG. 9K) and CDX2 (FIG. 9L) (log-transformed TPM) in human peri-implantation embryo and placenta trophoblasts.

FIG. 10A is a uMAP of in vitro trophoblast cells (EPSC-TSCs, BST-TSC and CT-TSC, and their derivatives) bulk RNA-seq data to the peri-implantation and placenta trophoblast clusters in FIG. 8B. In vitro cells are highlighted in light blue. BST-TSC and CT-TSC are derived from the human blastocyst and placenta cytotrophoblasts as previously reported. BST-STB, CT-STB, and EPSC-STB, and BST-EVT, CT-EVT, and EPSC-EVT are derived from the respective TSCs. FIG. 10B is a uMAP of the expression of ACE2 in peri-implantation and placenta trophoblast scRNAseq clusters. ACE2 is expressed in many PI-STBs.

FIGS. 11A-11D are scatter plots showing the positive Pearson correlations of CD46 (FIG. 11A), CGB5 (FIG. 11B), ENG (FIG. 11C) and CSH2 (FIG. 11D) with ACE2 expression. Linear regression line is drawn in black dashed line. Cell density is represented by the darkness of gray. FIGS. 11E and 11F are violin plots showing ACE2 (FIG. 11E) and TMPRSS2 (FIG. 11F) expression levels (log-transformed TPM) detected in scRNAseq dataset among different lineages at different stages in human peri-implantation embryo and placenta tissue. FIG. 11G is a plot for ACE2 and TMPRSS2 expression levels in ACE2-positive cells, categorized according to stage and cell lineage. Dots are colored according to the mean expression value in each category and dot size indicates the percentage of ACE2- or TMPRSS2-positive cells from each category that expresses ACE2. Only PI-STBs express high levels of both ACE2 and TMPRSS2. FIGS. 11H and 11I are bar graphs of the RNA-seq expression of ACE2 (FIG. 11H) and TMPRSS2 (FIG. 11I) in EPSC-TSCs and the differentiated subtype cells. STB-D6 and EVT-D8 are differentiated cells from EPSC-TSCs on day 6 and day 8, respectively. Cpm was used to normalize the signal.

FIG. 12A is a scaled expression of TMPRSS2, BSG and AXL in human peri-implantation embryo and placenta trophoblasts. FIGS. 12B and 12C are graphical representation of the RNA-seq signal expression levels of ACE2 (FIG. 12B) and TMPRSS2 (FIG. 12C) in hEPSCs and hEPSC derived hTSC and trophoblast subtypes.

FIG. 13A is a schematic of the experiment workflow of SARS-CoV-2 infection of trophoblasts. Cells were co-cultured with SARS-CoV-2 at 0.1 MOI for 2 hours followed by PBS wash and were subsequently incubated in fresh trophoblast media for 24, 48, or 72 hours. The supernatants and cell lysates were collected for viral genome detection and viral protein analysis. All the virus infection experiments were repeated at least three times. FIG. 13B is a line graph of the RT-qPCR detection of SARS-CoV-2 genome copy numbers (per mL) in the supernatants of virus-co-cultured hEPSCs and EPSC-TSCs at different time points. Datapoints are mean and SD from three independent experiments. ***p<0.001 (two-tailed unpaired Student's t-test). FIG. 13C is a bar graph of the RT-qPCR detection of SARS-CoV-2 genome copy number in cell lysates of 2 h.p.i. and 48 h.p.i. hEPSCs and EPSC-TSCs. Data are mean±s.d.n=3. Statistical analysis was performed using unpaired Student's t-test, ns: not significant; ***P<0.001. FIG. 13D is a dot plot showing the percentages of SARS-CoV-2 N protein positive EPSC-TSCs from the quantification of 36 random immunofluorescence staining images from the immunofluorescence staining of 24 h.p.i. BST-TSCs for SARS-CoV-2 N protein. Error bar: mean and standard error (SEM). FIGS. 13E and 13F are bar graphs of the RT-qPCR quantification of SARS-CoV-2 genome copy number in supernatants (FIG. 13E) and cell lysates (FIG. 13F) of 2, 24, 48 and 72 h.p.i. EVTs. Cells were submitted for SARS-CoV-2 co-incubation on day 8 of EPSC-TSC differentiation toward EVTs. Data are mean±s.d. n=3. Statistical analysis was performed using unpaired Student's t-test, ***P<0.001. FIG. 13G is a dot plot of the percentages of SARS-CoV-2 N protein positive EVTs from the immunofluorescence staining of 48 h.p.i. EVTs for SARS-CoV-2 N protein. Error bar: mean and standard error (SEM). n=36, quantification of 36 random immunofluorescence staining images. FIG. 13H is a line graph of the RT-qPCR detection of SARS-CoV-2 genome copy numbers (per mL) in the supernatants of virus-co-cultured EPSC-TSCs and STBs. The STBs were differentiated from EPSC-TSCs for 4 days (STB-D4) before SARS-CoV-2 infection. Datapoints are mean and SD from three independent experiments. ***p<0.001 (two-tailed unpaired Student's t-test). FIG. 13I is a bar graph of the RT-qPCR detection of SARS-CoV-2 genome copy number in cell lysates of 2 h.p.i. and 48 h.p.i. hEPSCs and EPSC-TSCs. Data are mean±s.d. n=3. Statistical analysis was performed using unpaired Student's t-test, ns: not significant; ***P<0.001. FIG. 13J is a pie chart of the proportion of mono-, bi- and multi-nucleated cells that were SARS-COV-2 infected cells. FIG. 13K is a dot plot of the quantification of percentages of mono-, bi- and multi-nucleated cells in STB-D2. Error bar, mean and standard error (SEM). n=40, quantification of 40 random immunofluorescence staining images, total 6734 cells. FIG. 13L is a dot plot showing the quantification of the percentages of CGB+ in STB-D2. Error bar, mean and standard error (SEM). n=8, quantification of 8 random immunofluorescence staining images, total 5820 cells. FIGS. 13M and 13N are bar graphs of the RNAseq analysis of expression of STB genes CD46 (FIG. 13M) and CGB5 (FIG. 13N) in EPSC-TSCs and EPSC-TSC differentiation toward STBs at different time points. FIG. 13O is a bar graph of the ACE2 and TMPRSS2 expression in naïve-TSCs that were infected by SARS-CoV-2. FIG. 13P is a graph showing the RNA signal of the ACE2 expression in STB-D2, STB-D4, STB-D6, and EPSC-TSCs.

FIG. 14A is a barplot for expression (cpm) of ACE2 and TMPRSS2 in hEPSC, EPSC-TSC and STBs and EVTs differentiated from EPSC-TSCs at indicated time points. FIG. 14B shows detection of ACE2 protein in EPSC-TSCs and during their differentiation toward STBs. ACE2 expression is rapidly induced and at high levels in STB-D2 (day 2) and onward. FIG. 14C shows RT-qPCR detection of SARS-CoV-2 genome copy numbers (per mL) in the supernatants of virus-co-cultured EPSC-TSCs and STBs. The STBs were differentiated from EPSC-TSCs for 4 days (STB-D4) before SARS-CoV-2 infection. Data are mean±s.d. n=3. Statistical analysis was performed using unpaired Student's t-test, ***P<0.001. FIG. 14D is a bar graph of the RNAseq analysis of SARS-CoV-2 genes (E, M, N, S) in cell lysates of 48 h.p.i. eSTBs (STB-D2). FIG. 14E is a dot plot of the quantification of the percentages of early STB marker CD46 in 24 h.p.i. eSTBs (STB-D2). Values are mean+SEM. RT-qPCR detection of SARS-CoV-2 genome copy numbers (per mL) in the supernatants of virus-co-cultured EPSC-TSCs and eSTBs (STB-D2). Supernatants were harvested at several time points post co-incubation. Data are mean±s.d. n=3. Statistical analysis was performed using unpaired Student's t-test, ***P<0.001. FIG. 14F is representative immunofluorescence images of 24 h.p.i. EPSC-TSCs and eSTBs (STB-D2) stained for SARS-CoV-2 N protein. FIG. 14G is plaque formation assay to detect SARS-CoV-2 virus in the supernatants from SARS-CoV-2-inoculated STB-D2 (48 h.p.i.). Data were obtained from three (n=3) independent batches of STB-D2. Datapoints are shown as mean±SD from three independent experiments. ***PC 0.001 (two-tailed unpaired Student's t-test). PFUs: plaque forming units. FIG. 14H is shows the quantification of CGB+ cells at STB-D2. FIG. 14I is dot plot showing the percentage of mono-, bi- and multi-nucleated cells that were SARS-COV-2 infected cells. FIG. 14J shows a bubble plot for RNAseq analysis of SARS-CoV-2 genes (E, M, N, S) in the cell lysates of 48 h.p.i. eSTBs (STB-D2). FIGS. 14K and 14L are bar plots of RT-qPCR detection of SARS-CoV-2 gene expression in supernatant (FIG. 14K) and cell lysates (FIG. 14L) of the infected STBs at 2, 24, 48 and 72 h.p.i.

FIG. 15A is a schematic of the experimental flow of eSTB generation for SARS-CoV-2 infection and sample collection for RNAseq. eSTBs used here are STB-D2. Samples were collected at 24 h.p.i. (STB-D3 infection) and 48 h.p.i. (STB-D4 infection) with three replicates. FIG. 15B and FIG. 15C are volcano plots of gene expression in infected STB-D3 and STB-D4. Significantly upregulated genes are labelled in red and significantly downregulated genes are labelled in blue. Horizontal red dash line marks adjusted P-value (Wald test) 0.05 and vertical lines marks expression fold change of 1.5. FIGS. 15D-15G are gene set enrichment analysis (GSEA) for cell cycle related genes including those of G2-M transition in virus infected STB-D3 and STB-D4 cells. Red lines are upregulated genes, blue lines are downregulated genes. FIGS. 15D-15G are graphs of the gene set enrichment analysis (GSEA) for cell cycle related genes including those of G2-M transition in virus infected STB-D3 and STB-D4 cells. Red lines are upregulated genes, blue lines are downregulated genes. FIG. 15H is a scatter diagram of transcriptomic analysis of transposable elements (TEs) after virus infection. Differentially expressed TEs (P<0.05, Wald test) are labelled with colours while none-differentially expressed TEs are labelled with grey colour. Expression levels of TEs used for x-axis are from DESeq2 results. Data of STB-D3 and STB-D4 are combined in this analysis. FIG. 15I is an RNAseq signal of HERV-W in infected STB-3 and STB-4 and the mock infection control cells. Library size was used to normalize the read counts. FIG. 15J shows PI-TB to PI-STB subpopulations colored by machine learning predicted pseudotime. X-axis is the predicted pseudotime, and y-axis is diffusion pseudotime computed using SCANPY. Grey dashed arrow describes the linear regression relationship between the predicted pseudotime and the diffusion pseudotime. FIG. 15K is a pseudotime analysis depicting PI-TB to PI-STB development trajectory. Dark purple indicates earlier pseudotime, light yellow indicates STB development. PI-TB to PI-STB pseudotime highlighted in the merged in vivo scRNA datasets. Black dashed arrow indicates the imputed direction of differentiation. FIG. 15L is a heatmap for correlation between infected STBs and mock infection STBs. Unexpressed genes are filtered out and cpm is used to calculate Pearson correlation coefficient. FIGS. 15M and 15N are the Venn plots for upregulated genes and downregulated genes after SARS-CoV-2 WT infection in STB-D3 and STB-D4. FIGS. 15M and 15N shows upregulated genes and downregulated genes after SARS-CoV-2 WT infection in STB-D3 and STB-D4.

FIG. 16A is the heatmap for correlation between infected STBs and mock infection STBs. Unexpressed genes are filtered out and cpm is used to calculate Pearson correlation coefficient. FIG. 16B is a bubble plot for ACE2, TMPRSS2, genes of SARS-CoV-2, innate immune response, TNF signaling pathway, interleukin signaling pathway and TNF-α/NF-κB signaling. Label color is according to z-score and bubble sizes are proportional to expression levels. FIG. 16C is the plot of the top 50 up-regulated and top 50 down-regulated genes in STB-D3 and STB-D4 after virus infection. Genes are sorted by −log 10 (p value). Z-score of log 2 transformed cpm was used. Red, upregulated genes; blue, downregulated genes. FIGS. 16D-16E are gene set enrichment analysis (GSEA) for cell cycle (FIG. 16D) and apoptosis (FIG. 16E) pathways in STB-D3, STB-D4 after SARS-CoV-2 WT infection. FIG. 16F is gene set enrichment analysis (GSEA) for apoptosis pathways in Vero E6 cells after SARS-CoV-2 WT infection. FIG. 16G is images of SARS-CoV-2 WT infected Vero E6 cells and eSTBs after 72 h.p.i. and representative immunofluorescence staining images of TUNEL cell apoptosis detection assay. Vero E6 cell almost detached and showed strong FITC fluorescence after 72 h.p.i. indicating obvious apoptosis. eSTB showed weak FITC fluorescence after 72 h.p.i. indicating low apoptosis. White arrows point to apoptotic cells. FIG. 16H shows a GO and KEGG analysis for shared upregulated and downregulated genes in SARS-CoV-2 WT infected STB-D3 and STB-D4. FIG. 16I shows a bubble plot for host factors ACE2, TMPRSS2, CTSL, CTSV, CTSB and genes of SARS-CoV-2, innate immune response, TNF signaling pathway, interleukin signaling pathway and TNF-α/NF-κB signaling mock and infected eSTBs. Bubble colors is in accordance with z-score and bubble sizes are proportional to expression levels.

FIG. 17A is a schematic of the experimental design for drug treatment. eSTBs (STB-D2) were submitted for SARS-CoV-2 or MERS-CoV infection at 0.1 MOI for one hour followed by incubation with or without remdesivir and GC376. Cells were collected at 48 h.p.i. for analysis. FIG. 17B is dose-dependent inhibition of SARS-CoV-2 WT/Delta/Omicron by remdesivir/GC376 in EPSC-derived eSTB cells. Viral load reduction assay was performed to evaluate the inhibitory effect of the indicated drugs against SARS-CoV-2 WT and Delta and Omicron variants infection in EPSC-eSTB cells. Viral copy in the supernatant were quantified by qRT-PCR method. Data are shown as mean±SEM. n=3. Statistical analysis was performed using Two-way ANOVA; (ns, not significant, *P<0.05; **P<0.01, ***P<0.001, ****P<0.0001). FIGS. 17B and 17C are bar graphs showing the viral copy number in EPSC-eSTB in the presence or absence of remdesivir 48 h.p.i. with either SARS-CoV-2 (FIG. 17B) or MERS-CoV (FIG. 17C) cells. FIGS. 17D and 17E are bar graphs showing the viral copy number in EPSC-eSTB in the presence or absence of GC376 48 h.p.i. with either SARS-CoV-2 (FIG. 17C) or MERS-CoV (FIG. 17E) cells. FIG. 17F is mapping of 48 h.p.i. EPSC-eSTB in the presence or absence of remdesivir (10 μM) or GC376 (10 μM) against in vivo pseudotime trajectory. Pseudotime increases from bottom left to top right, as shown in FIG. 4I. The cells with different treatment were colored: red for infected cells without any treatment, green for mock (non-infected cells, no drug treatment), light blue for infected cells treated by remdesivir, and dark blue for infected cells treated by GC376. FIGS. 17G and 17H are box plots for up-regulated (FIG. 17G) and down-regulated (FIG. 17H) genes in 48 h.p.i. eSTBs, with or without SARS-CoV-2 infection, and in the presence or absence of remdesivir or GC376. Statistical significance was calculated by Wilcoxon test using mean of expression value. FIG. 17I is a map of the 48 h.p.i. eSTBs (in the presence or absence of remdesivir or GC376) against in-vivo pseudotime trajectory. Pseudotime increases from bottom left to top right, as shown in FIG. 15K. The cells with different treatment were colored: red for infected cells without any treatment, green for mock (non-infected cells, no drug treatment), light blue for infected cells treated by remdesivir, and dark blue for infected cells treated by GC376. FIGS. 17J-17O are graphs showing treatment of delta and omicron infected cells treated with vehicle, remdesivir or GC376 respectively.

FIGS. 18A-18H are bar graphs showing the RT-qPCR results of trophoblast transcription factor genes (TEAD4 (FIG. 18E), CDX2 (FIG. 18F), ITGA6 (FIG. 18A) and GATA3 (FIG. 18B)), STB genes (ERVW-1 (FIG. 18C) and CGB (FIG. 18G)) and EVT genes (ITGA1 (FIG. 18H) and ITGA5 (FIG. 18D)) in EPSC-TSCs and trophoblast organoids (Data are mean+SD, n=3 independent replicates from 3 separate sample extracts. **p<0.01; ***p<0.001 (two-tailed unpaired Student's t-test). Gene expression levels were normalized to GAPDH using the ΔCt method. FIGS. 18G-18H is expression of STB genes (ERVW-1 (FIG. 18C) and CGB (FIG. 18G) in STBs, EPSC-TSCs and trophoblast organoids by RT-qPCR. Data are mean±s.d. n=3. Statistical analysis was performed using unpaired Student's t-test, ***P<0.001, Gene expression levels were normalized to GAPDH using the ΔCt method. FIG. 18I is the heatmap showing EPSC-ORGs expression of both TSC and STB trophoblast genes, similar to trophoblast organoids generated from CT-TSCs (CT29, CT30 and BTS11). Z-score of cpm was used and shown as heatmap signatures. FIG. 18J is barplot for expression (cpm) of ACE2 and TMPRSS2 in EPSC-TSC/STB/EVT and EPSC-TSC generated trophoblast organoids (EPSC-ORG). Right panel: RT-qPCR detection of SARS-CoV-2 genome copy numbers (per mL) in the supernatants of virus-co-cultured EPSC-TSCs, eSTBs (STB-D2) and EPSC-ORGs. Supernatants were harvested at several time points post co-incubation. Data are mean±s.d. n=3. FIG. 18K is a micrograph image of CD46, eCadherin, TFAP2A, aYAP1 and CGB expression in trophoblast organoids. FIG. 18L-18O are plots showing gene expression changes in trophoblast organoids.

FIG. 19A is a schematic of the experiment work flow of hEPSCs differentiation towards trophoblasts by the TGF-β inhibitor SB-431542 (SB43) treatment. This method directly produces trophoblasts, primarily STBs, from hEPSCs. FIG. 19B is a bar graph of the RT-qPCR results of genes encoding ACE2 and TMPRSS2 in hEPSCs and the differentiated cells on day 4 and day 9. hEPSCs have no ACE2 expression. nd: undetectable. (Data are mean+SD, n=3 independent replicates from 3 separate sample extracts. **p<0.01; ***p<0.001 (two-tailed unpaired Student's t-test). Gene expression was normalized to GAPDH using the ΔCt method. FIG. 19C is a line graph of SARS-CoV-2 replication kinetics in cells differentiated from hEPSCs compared to Vero E6 and Caco2 cells. Caco2 cell, an immortalized cell line derived from human colorectal adenocarcinoma and is often used in infection studies of SARS-CoV-2. All cells were infected with SARS-CoV-2 at 0.1 MOI. Viral supernatant samples were harvested at 2, 24, 48 and 72 h.p.i. for RT-qPCR of viral RNA load. (Data are mean+SD, n=3 independent replicates from 3 separate experiments. ***p<0.001 (two-tailed unpaired Student's t-test). FIG. 19D is expression of pluripotency and various cell lineage genes in normal and ACE2-KO hEPSCs FIG. 19E shows flow cytometry quantification of pluripotency markers SSEA3 and TRA-1-60 on normal and ACE2-KO hEPSCs. FIG. 19F shows expression of trophoblast genes in SB43-treated hEPSCs on day 9 by RT-qPCR. Data are mean±s.d. n=3. Statistical analysis was performed using unpaired Student's t-test, **P<0.001, ***P<0.001, Gene expression levels were normalized to GAPDH using the ΔCt method. FIG. 19G is RT-qPCR analysis of genes encoding ACE2 and TMPRSS2 in hEPSCs and the differentiated cells on day 4 and day 9. hEPSCs have no ACE2 expression. nd: undetectable. (Data are mean±SD, n=3 independent replicates from 3 separate sample extracts. **P<0.01; ***P<0.001 (two-tailed unpaired Student's t-test). Gene expression was normalized to GAPDH using the ΔCt method FIG. 19H Quantification of supernatant viral RNA loads of SB43-treated (day 4) normal and ACE2-KO EPSCs. Cells were infected with SARS-CoV-2 at 0.1 MOI and supernatant samples were harvested at 2, 24, 48 and 72 h.p.i. (Data are mean±SD, n=3 independent replicates from 3 separate experiments. ***P<0.001 (two-tailed unpaired Student's t-test). FIG. 19I SARS-CoV-2 viral genome quantitation and expression of ACE2 and TMPRSS2 in normal versus SB43-treated ACE2-KO EPSCs at day 4. (Data are mean±SD, n=3 independent replicates from 3 separate sample extracts. nd, not detectable. Gene expression was normalized to GAPDH using the ΔCt method. FIG. 19J shows sanger sequencing of the mutant PCR fragment reveals a 148 bp deletion between the two CRISPR gRNAs in exon 2 as expected.

FIG. 20A is a bar graph of the RT-qPCR quantification of naïve marker gene expression levels in naïve stem cells compared to that in hEPSCs. Data are mean±s.d. n=3. FIGS. 20B and 20C are bar graphs of RT-qPCR quantification of hTSC marker gene expression levels in naïve-TSC compared to that in EPSC-TSC. Data are mean±s.d. n=3. RT-qPCR quantification of ISL1 and MUC16 gene expression levels in naïve stem cells and naïve-TSCs. Data are mean±s.d. n=3. FIG. 20D is representative phase-contrast image of human naïve stem cells (H1) cultured on MEFs in PXGL condition. FIG. 20E shows representative immunofluorescence staining of naïve pluripotency markers KLF17 and CD75 in naïve stem cells. FIG. 20F is representative phase-contrast image of naïve stem cell-derived hTSCs (naïve-TSCs). FIG. 20G is representative immunofluorescence staining of TFAP2C, TP63 and CK18 in naïve-TSCs. Right panel: Percentages of SARS-CoV-2 N protein positive naïve-TSCs, respectively. Error bar: mean and standard error (SEM). n=60, quantification of 60 random immunofluorescence staining images.

FIG. 21A is a dot plot of the quantification of naïve-eSTB for SARS-CoV-2 N protein. FIGS. 21B-21D are line graphs showing the comparison of SARS-CoV-2 replication kinetics in naïve-eSTB and BST-eSTB cells. Cells were infected with WT, the Delta variant or Omicron variant using 0.01 MOI. Cell culture supernatant were collected at the indicated time-points and subject to viral load detection using RT-qPCR method. Data are mean±s.d. n=3. FIGS. 21E-21J are bar graphs of the viral load reduction assay was performed to evaluate the inhibitory effect of the indicated drugs against SARS-CoV-2 WT and Delta and Omicron variants infection in naïve-eSTB cells. Viral copy in the supernatant were quantified by RT-qPCR method. The results are shown as the mean±SEM. Statistics analysis by Two-way ANOVA. (ns, not significant, *P<0.05; **P<0.01, ***P<0.001, ****P<0.0001).

FIG. 22 is a schematic of studying the susceptibility to SARS-CoV-2 and MERS-CoV of human trophoblasts derived from EPSCs, naïve stem cell, and placenta/blastocyst. TSCs established from EPSCs, naïve stem cells, human blastocysts (BST-TSC) or placenta cytotrophoblast (CT-TSC) all differentiate to trophoblast subtypes STBs and EVTs. EPSC-TSCs can also generate 3D trophoblast organoids. Under TGF-β inhibition human EPSCs can directly differentiate to trophoblasts for SARS-CoV-2 infection. Knocking out ACE2 eliminates the infection demonstrating that SARS-CoV-2 infects human trophoblasts via ACE2. The identified eSTBs (early STBs) of the present invention are highly susceptible to SARS-CoV-2 (WT, Delta and Omicron) and MERS-CoV infection and may serve as an improved physiologically relevant cell source for coronavirus research, antiviral drug evaluation and vaccine development.

### DETAILED DESCRIPTION OF THE INVENTION

### I. Definitions

The term “stem cell” refers to an undifferentiated cell which is capable of proliferation, self-renewal and giving rise to more progenitor or precursor cells having the ability to generate many mother cells that can in turn give rise to differentiated, or differentiable, daughter cells. The daughter cells can for example be induced to proliferate and produce progeny cells that subsequently differentiate into one or more mature cell types, while also retaining one or more cells with parental developmental potential. All stem cells have three important properties: (1) they are unspecialized, (2) are capable of continuous division and self-renewal, and (3) are capable of differentiation into specialized cells. According to the developmental potential, there are totipotent stem cells (zygote), pluripotent stem cells (e.g., ESC, iPSC) and unipotent stem cells (e.g., neural stem cells, and muscular stem cell). Thus, stem cells include embryonic stem cells, pluripotent stem cells, and unipotent stem cells of various types and from various sources.

The term “pluripotency” (or pluripotent), as used herein refers the potential of a cell to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system). “Pluripotent stem cells” are cells which exhibit an undifferentiated phenotype and are potentially capable of differentiating into any fetal or adult cell type of any of the three germ layers (endoderm, mesoderm, and endoderm). A pluripotent stem cell is distinct from a totipotent stem cell and generally cannot give rise to extraembryonic cell lineages. The population of pluripotent stem cells may be clonal i.e., genetically identical cells descended from a single common ancestor cell. Pluripotent stem cells are classified into naïve and primed based on their growth characteristics in vitro and their potential to give rise to all somatic lineages and the germ line in chimeras. ESCs that are derived from the inner cell mass (ICM) of preimplantation embryos represent naïve pluripotent stem cells (PSCs). Naïve stem cells have an unlimited self-renewal capacity when grown under appropriate conditions and are able to differentiate into tissues of all three germ layers in vitro. In addition, when injected back into the early embryos, naïve stem cells contribute to all somatic lineages including the germline. This ability to generate chimeras is indicative of their pluripotency in vivo. On the other hand, epiblast stem cells (EpiSCs) that are derived from the epiblast of the post-implantation embryo typify the “primed” state. Also included in this category are hESCs and hiPSCs that resemble closely the EpiSCs, even though they are isolated from preimplantation embryos. Like the naïve PSCs, the primed PSCs also have unlimited potential to self-renew and differentiate into three germ layers in vitro, but are limited in their pluripotency in vivo, as they cannot give rise to germline chimeras.

The term “induced pluripotent stem cells” refers to pluripotent cells derived from a donor cell that is not pluripotent, i.e., a multipotent or differentiated cell, by engineering somatic cells to express one or more markers of pluripotency including POU4F1/OCT4 (Gene ID; 5460) in combination with, but not restricted to, SOX2 (Gene ID; 6657), KLF4 (Gene ID; 9314), cMYC (Gene ID; 4609), NANOG (Gene ID; 79923), LIN28/LIN28A (Gene ID; 79727)). The expression can be induced for example by forced gene expression or using small molecules, small RNAs, non-integrating gene expression vectors, or proteins.

“Differentiation” or “differentiate” as used herein refers to the process whereby stem cells transform into more specialized cell types and can perform new functions through the expression of new genes, mRNA, and proteins. Differentiation involves the deactivation of some genes and the activation of a new set of genes.

“Extraembryonic cells” as used herein refers to cells and/or tissue that grow from the fertilized egg but that do not remain as part of the developing embryo. Typically, the extraembryonic tissues and/or cells are placental trophoblasts which nourish and protect the fetus.

The term “expanded potential stem cells” or “EPSCs” as used herein refer to pluripotent stem cells with an improved ability to generate extraembryonic lineages. The EPSCs as described herein can be derived from preimplantation embryos of multiple species of mammals, including but not limited to mice, humans, pigs, and cows. For example, in the mouse, EPSCs demonstrate the molecular signature similar to 4-8 cell stage embryos. EPSCs retain the development potential to all extraembryonic and embryonic cell lineages (or totipotency features). EPSCs can also be derived by reprogramming somatic cells using defined sets of exogenous factors and by culturing conventional ESCs in EPSC culture media. A single EPSC contributes to both the embryo proper and the trophectoderm lineages. Trophoblast stem cell (TSC) lines, extraembryonic endoderm stem cells (XEN), and ESCs could be directly derived from EPSCs in vitro. Mononuclear cytotrophoblasts (CTBs) are derived from the trophectodermal layer and are considered to be trophoblast stem cells (TSCs). TSCs are undifferentiated and proliferative population of cells that can differentiate into either syncytiotrophoblasts (STBs) or extravillous trophoblasts (EVTs).

“Variant” or “strain” as used herein refers to a viral genome (genetic code) that may contain one or more mutations. A viral variant may have different functional properties to the original virus. When a virus replicates, it does not always manage to produce an exact copy of itself. This means that, over time, the virus may start to differ slightly in terms of its genetic sequence. Any changes to the viral genetic sequence during this process is known as a mutation and viruses with new mutations are sometimes called variants. Variants can differ by one or multiple mutations.

The term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCR, in situ hybridization, Western blotting, and immunostaining such as FACS.

The term “expressing” also represented as “+” means, with respect to a cell protein level, detectable protein expression compared to a cell that is not expressing the protein, for example as measured by FACS analysis.

The term “culturing” as used herein incubating and/or passaging cells in an adherent, suspension or 3D culture. As used herein, the term “adherent culture” refers to a cell culture system whereby cells are cultured on a solid surface, which may in turn be coated with an insoluble substrate that may in turn be coated with another surface coat of a substrate, such as those listed below, or any other chemical or biological material that allows the cells to proliferate or be stabilized in culture. The cells may or may not tightly adhere to the solid surface or to the substrate.

The term “contacting” or “culturing . . . with” is intended to include incubating the component(s) and the cell/tissue together in vitro (e.g., adding the compound to cells in culture) and the step of “contacting” or “culturing . . . with” can be conducted in any suitable manner. For example, the cells may be treated in adherent culture, in suspension culture, or in 3D culture; the components can be added temporally substantially simultaneously (e.g., together in a cocktail) or sequentially (e.g., within 1 hour, 1 day or more from an addition of a first component). The cells can also be contacted with another agent such as a growth factor or other differentiation agent or environments to stabilize the cells, or to differentiate the cells further and include culturing the cells under conditions known in the art. The population of cells can be enriched using different methods such as methods based on markers such as cell surface markers (e.g., FACS sorting etc.).

The term “inhibitor” means a selective inhibitor, for example, of a pathway or signaling molecule. An inhibitor or antagonist of a molecule (e.g., TGFβ inhibitor) can inhibit one or more of the activities of the naturally occurring form of the molecule. For example, a TGFβ inhibitor is a molecule that selectively inhibits TGFβ signaling.

The term “subject” as used herein includes all members of the animal kingdom including mammals such as and including a primate such as human, monkey or ape, domestic pets, livestock, and laboratory animals.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to a subject.

The term “treatment” as applied to a subject, refers to an approach aimed at obtaining beneficial or desired results, including clinical results and includes medical procedures and applications including for example pharmaceutical interventions, surgery, radiotherapy, and naturopathic interventions as well as test treatments for treating joint/bone disorders. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable.

The terms “administering”, “implanting” and “transplanting” are used interchangeably in the context of delivering cells tissues and/or products described herein into a subject, by a method or route which results in at least partial localization of the introduced cells at a desired site. The cells can be implanted directly to a joint, or alternatively be administered by any appropriate route which results in delivery to a desired location in the subject where at least a portion of the implanted cells or components of the cells remain viable.

“Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

The term “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., injury size/type, age, joint health, immune system health, etc.), the disease or disorder, and the treatment being administered. The effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

“Excipient” is used herein to include a compound that is not a therapeutically or biologically active compound. As such, an excipient should be pharmaceutically or biologically acceptable or relevant, for example, an excipient should generally be non-toxic to the subject. “Excipient” includes a single such compound and is also intended to include a plurality of compounds.

### II. Compositions

A stem cell-based system containing early Syncytiotrophoblasts (eSTBs) with desirable features for culturing coronavirus particles are disclosed. The disclosed eSTBs can be well suited for culturing high titers of coronaviruses and/or coronavirus particles, evaluating drug therapies, cell and tissue engineering, gene editing, and viral research. In exemplary forms, the disclosed eSTBs are suitable for investigating the susceptibility of trophoblast cells to coronavirus infections and for screening compounds for the treatment and prevention of coronavirus infections. Importantly, the disclosed eSTBs have significantly longer survival rates compared to existing cells e.g. Vero E6 cells following viral infection as mechanistically confirmed by morphological and molecular assessments provided in the non-limiting examples. Cells for deriving the eSTBs are also disclosed. Kits containing agents for producing the eSTBs according to methods, and one or more excipients are also provided herein.

A. Cells

1. Trophoblast Stem Cells

Trophoblasts are the multipotent precursors of the differentiated cells of the placenta and are crucial for the correct function of the placenta. Trophoblast stem cells (TSCs) as described herein are a proliferative population that can differentiate into the STBs and extravillous cytotrophoblasts (EVTs). In some forms, the TSCs as described herein are capable of indefinite proliferation in vitro in an undifferentiated state. In some forms, the described TSCs maintain the potential multilineage differentiation capabilities. In some forms, the TSCs can be induced to differentiate into cells of the trophoblast lineage.

In some forms, the derived TSCs are identified by screening the TSC colonies for epithelial cobblestone-shaped TSC-like morphology.

In some forms, the undifferentiated TSCs are characterized by expression of genetic markers of TSC cells. In some forms, the undifferentiated TSC cells may be further characterized by negative expression of marker genes for TSC-derivative cells. Typically, the derived TSCs are identified by screening the TSC colonies for expression of a TSC-typical molecular signature. In some forms, the TSC-typical molecular signature is increased TSC factors. In some forms, the increased TSC factors are TFAP2C, TP63, CK18, GATA2, GATA3, ELF5, TEAD4, and/or KRT7, or any combination thereof.

In some forms, the TSC-typical molecular signature is increased trophoblast-specific miRNAs. In some forms, the increased trophoblast-specific miRNAs are has-miR-517c-3p, 517-5p, 525-3p, and/or 526b-3p or any combination thereof.

In some forms, the TSC-typical molecular signature is decreased HLA class I molecules, and/or decreased AME genes. In some forms, the decreased HLA class I molecules are HLA-A and/or HLA-B. In some forms, the decreased AME genes are CDX2, MUC16, GABRP, ITGB6, and/or VTCN1.

In some forms, the TSCs may be human TSCs or non-human TSCs. In some forms, non-human TSCs are mammalian TSCs, such as for example bovine TSCs, ovine TSCs, porcine TSCs, canine TSCs, feline TSCs, equine TSCs, or primate TSCs. In preferred forms, the TSCs are human TSCs.

In some forms, the disclosed TSCs are established from pluripotent stem cells, including but not limited to EPSCs, naïve stem cells, and primed stem cells. In some forms, the disclosed TSCs are established from derivatives of pluripotent stem cells following genetic manipulation or gene-editing.

In some forms, the disclosed TSCs are established from preimplantation embryos and/or placental tissues. In some forms, the disclosed TSCs are established from derivatives of preimplantation embryos and/or placental tissues following genetic manipulation or gene-editing.

2. Early Syncytiotrophoblasts (eSTBs)

The syncytiotrophoblasts are a continuous, specialized layer of epithelial cells. They cover the entire surface of villous trees and are in direct contact with maternal blood. The surface area of syncytiotrophoblasts is about 5 square meters at 28 weeks' gestation and reaches up to 11 to 12 square meters at term and facilitate nutrient circulation between the embryo and the female. At maturity, the STBs are multinucleate.

It has been established that early syncytiotrophoblasts (eSTBs) generated from TSCs have enriched transcriptomic features of peri-implantation trophoblasts, express high levels of angiotensin-converting enzyme 2 (ACE2) and are productively infected by SARS-CoV-2 and its Delta and Omicron variants producing new virions. Typically, the eSTBs used to generate coronavirus particles are mononucleated cells and are not multi-nucleated or mature cells.

In some forms, the derived eSTBs are isolated by selecting cells expressing eSTB-like morphology, cells expressing an eSTB-like molecular signature, or cells expressing both eSTB-like morphology and an eSTB-like molecular signature. In some forms, eSTB-like molecular signature includes one or more increased STB markers, wherein the increased STB markers are GCM1, β chorionic gonadotrophin 3 gene (CGB3), CGB5, CD46, ENG, and/or CSH2. In some forms, the eSTB-like molecular signature does not include increased trophoblast progenitor transcription factor TP63, and/or properly folded and secreted β-hCG hormone. In some forms, the eSTB-like molecular signature includes increased CD46 expression and increased SSEA4 expression.

Typically, the eSTBs used in the disclosed compositions for culturing coronavirus particles have one or more coronavirus susceptible markers. The disclosed eSTBs have significantly longer survival rates compared to existing cells e.g., Vero E6 cells following viral infection as mechanistically confirmed by morphological and molecular assessments provided in the non-limiting examples. In preferred forms, the coronavirus susceptible markers are increased ACE2 and/or increased TMPRSS2. In an exemplary form, the coronavirus susceptible marker ACE2 is identified via RT-qPCR using the primers SEQ ID NO:2 and SEQ ID NO:52, SEQ ID NO:3 and SEQ ID NO:53, or SEQ ID NO:96 and SEQ ID NO:97. In an exemplary form, the coronavirus susceptible marker ACE2 is identified via cell staining techniques such as for example immunofluorescence staining. In other preferred forms, the coronavirus susceptible marker TMPRSS2 is identified via RT-qPCR using the primers SEQ ID NO:4 and SEQ ID NO:54. In some forms, eSTBs are identified via cell staining techniques such as for example immunofluorescence staining of CD46, SSEA4, and are present as predominantly mononucleated and not multinucleated cells when stained by DAPI. In some forms, during TSC differentiation to STBs, the eSTBs are abundantly present in the cultures from about day 2 to about day 4.

The eSTBs may be human eSTBs or non-human eSTBs. In some forms, non-human eSTBs are mammalian eSTBs, such as for example bovine eSTBs, ovine eSTBs, porcine eSTBs, canine eSTBs, feline eSTBs, equine eSTBs, or primate EP eSTBs. In preferred forms, the eSTBs are human eSTBs.

The disclosed eSTBs can be generated from TSCs of multiple stem cell sources. In some forms, the eSTBs can be generated from TSCs derived from naïve stem cells. In some forms, the eSTBs can be generated from TSCs derived from primed stem cells. In some forms, the eSTBs can be generated from TSCs derived from expanded potential stem cells or other types of pluripotent stem cells. In some forms, the eSTBs can be generated from TSCs established from preimplantation embryos or placental tissues. In some forms, the eSTBs can be generative from one or more derivatives of naïve stem cells, primed stem cells, pluripotent stem cells, expanded potential stem cells, preimplantation embryos or placental tissues following genetic manipulation or gene-editing.

B. Sources of Cells

The disclosed trophoblast stem cells (TSCs) can be obtained from various sources including but not limited to embryonic tissues, fetal tissues, adult tissues and differentiated somatic cells after they have been genetically reprogrammed (referred to as induced pluripotent stem cells (iPSCs). In some forms, the disclosed TSCs can be obtained from EPSCs, naïve stem cells, primed stem cells, and/or embryos or placenta. Generally, TSCs, regardless of the method of derivation, are capable of generating STBs and EVTs. STBs generated from these TSCs are generally susceptible to viral infections. For example, early STBs generated from the disclosed TSCs are highly susceptible to coronavirus infection and can be used for producing large quantities of coronavirus for vaccine production and antiviral discovery.

The TSCs and their derivatives, eSTBs and EVTs, are typically obtained by inducing pluripotent cells, or partially or completely differentiated cells obtained from a mammal. Non-limiting examples of mammals include rodents such as for example mice, rats, hamsters, and guinea pigs, lagomorphs such as for example rabbits and hares, ungulates such as for example, pigs, cows, goats, horses, and sheep, dogs and cats, and human primates such as monkeys, rhesus monkeys, cynomolgus monkeys, marmosets, orangutans, and chimpanzees. Preferably, the mammal is such as a human or non-human primate. Sources of cells for induction to TSCs and their derivatives include but are not limited to bone marrow, fibroblasts, fetal tissue (e.g., fetal liver tissue), peripheral blood, umbilical cord blood, pancreas, skin or any organ or tissue. A population of the TSCs and their derivatives, eSTBs and EVTs, may be produced as described herein by culturing a population of pluripotent cells in an expanded potential stem cell medium (EPSCM) to produce a population of EPSCs. Pluripotent cells are cells which exhibit an undifferentiated phenotype and are potentially capable of differentiating into any fetal or adult cell type of any of the three germ layers (endoderm, mesoderm, and endoderm). A pluripotent cell is distinct from a totipotent cell and generally cannot give rise to extraembryonic cell lineages. The population of pluripotent cells may be clonal i.e., genetically identical cells descended from a single common ancestor cell. In some forms, the pluripotent stem cells are embryonic stem cells (ESCs). In some forms, the pluripotent stem cells are non-embryonic stem cells, for example fetal and adult stem cells. In other forms, the pluripotent stem cells are induced pluripotent stem cells (iPSCs). In some forms, the pluripotent stem cells are not ‘primed’ pluripotent stem cells, such as epiblast stem cells (EpiSCs).

1. Expanded Potential Stem Cells

EPSCs have an expanded potential to differentiate into extraembryonic cell lines (trophoblasts and extraembryonic endoderm in the yolk sac) as well as cells of the embryo proper, which are derived from the epiblast of the blastocyst. EPSCs may be consistently produced from different pluripotent cell lines which are cultured in expanded potential stem cell media (EPSCM). EPSCs have been successfully differentiated into a range of stem cell types including EPSC-derived TSCs, and their derivatives eSTBs and EVTs as described herein. The EPSC-derived stem cells may also be induced to derive a range of cell types including pancreatic cells, neurons, and T-cells.

Typically, the EPSCs do not express ACE2. Thus, the EPSCs in the disclosed compositions are poorly infected by coronaviruses. In some forms, lack of coronavirus infection in the EPSCs is evidenced by little or no increases in viral genome in the supernatant or cell lysate. In some forms, lack of coronavirus infection in the EPSCs is evidenced by the lack of the presence of viral N protein as detected by immunofluorescence staining or RT-qPCR.

In some forms, the EPSCs may be human EPSCs or non-human EPSCs. In some forms, non-human EPSCs are mammalian EPSCs, such as for example bovine EPSCs, ovine EPSCs, porcine EPSCs, canine EPSCs, feline EPSCs, equine EPSCs, or primate EPSCs. In preferred forms, the EPSCs are human EPSCs.

2. Embryonic Stem Cells

Embryonic stem cells may be obtained using conventional techniques. For example, ESCs cells may be obtained from a cultured ESC cell line, for example a hESC line. Numerous cultured ESC lines are publicly available from repositories (e.g., NIH Human Embryonic Stem Cell Registry), such as CHB-1 to CHB-12, RUES1 to RUES3, HUES1 to HUES28, HUES45, HUES48, HUES49, HUES53, HUES62 to HUES66, WA01 (H1), WA07 (H7), WA09 (H9), WA13 (H13), WA14 (H14), NYUES1 to NYUES7, MFS5, and UCLA1 to UCLA3. Examples of suitable embryonic stem cell lines are described in Thomson J A et al Science 282: 1145-1147 (1998); Reubinoff et al. Nat Biotechnol 18:399-404 (2000); Cowan, C. A. et al. N. Engl. J. Med. 350, 1353-1356(2004), Gage, F. H., et al. Ann. Rev. Neurosci. 18 159-192 (1995); and Gotlieb (2002) Annu. Rev. Neurosci 25 381-407); Carpenter et al. Stem Cells. 5(1): 79-88 (2003). Potentially clinical grade ESCs are described in Klimanskaya, I. et al. Lancet 365, 1636-1641 (2005) and Ludwig, T. E. et al. Nat. Biotechnol. 24, 185-187 (2006). Suitable ESCs may be obtained for use without either destroying a mammalian embryo or using a mammalian embryo for an industrial or commercial purpose. For example, ESCs may be obtained by blastomere biopsy techniques (Klimanskaya (2013) Semin Reprod Med. 31(1):49-55; Cheung et al 2008, Klimanskaya et al Nature (2006) 444(7118)481-5).

In some forms, the embryonic pluripotent cells may be a pre-implantation embryo or individual blastocyst. When the pluripotent cells are human, they may be obtained without destruction of a human embryo using known techniques as discussed herein.

3. Naïve and/or Primed Stem Cells

The disclosed TSCs may be derived from naïve and/or primed stem cells. Naïve and primed pluripotent states can be functionally classified on the basis of their ability or failure to maintain self-renewal of the pluripotent state upon inhibition of MEK signaling, respectively. Naïve and primed states of pluripotency represent a continuum of configurations rather than a fixed individual state. Within the naïve and primed pluripotent states, different degrees of naivety or priming can be found on the basis of various characteristics. Naïve pluripotency refers to a pluripotent state that resembles the pre-implantation embryonic configuration. Primed pluripotency refers to a pluripotent state that resembles the post-implantation embryonic configuration.

4. Induced Pluripotent Stem Cells

iPSCs are pluripotent cells which are derived from non-pluripotent, differentiated ancestor or antecedent cells. Suitable ancestor cells include somatic cells, such as adult fibroblasts and peripheral blood cells. In some forms, the TSCs and their derivatives, eSTBs and EVTs, are indirectly derived from somatic cells. A “somatic cell” would be understood by one of ordinary skill in the art is any cell other than a gamete (sperm or egg), germ cell (cells that go on to become gametes), gametocyte or undifferentiated stem cell. The somatic cells can be obtained from tissue such as bone marrow, fetal tissue (e.g., fetal liver tissue), peripheral blood, umbilical cord blood, pancreas, skin or any organ or tissue. Following reprogramming of somatic cells into iPSCs, the TSCs and their derivatives, eSTBs and EVTs, can then be derived from the iPSCs.

In one form, the TSCs and their derivatives, eSTBs and EVTs, are derived from fibroblasts, adipose-derived cells, neural cells, or cells from the intestinal epithelium.

In another form, TSCs and their derivatives, eSTBs and EVTs, are obtained from induced neonatal (for example foreskin) or adult fibroblasts. However, TSCs and their derivatives, eSTBs and EVTs, can be obtained from other cell types including but not limited to somatic cells of hematological origin, skin derived cells, adipose cells, epithelial cells, endothelial cells, cells of mesenchymal origin, parenchymal cells (for example, hepatocytes), neurological cells, and connective tissue cells.

In some forms, the TSCs and their derivatives, eSTBs and EVTs, are effectively generated from tissue specimens such as small tissue biopsies or a few drops of blood.

Ancestor cells are typically reprogrammed by the introduction of pluripotency genes (or RNA encoding them) or their corresponding proteins into the cell, or by re-activating the endogenous pluripotency genes. The genes, RNA encoding them, or proteins may be introduced into the differentiated cells by any suitable technique, including plasmid or more preferably, viral transfection or direct protein delivery. Pluripotency genes include members of the Oct family, such as Oct or Sox family 4 (also known as oct3/4), Sox2 and Sox1. Other genes, for example Klf genes, such as Klf-1, -2, -3, -4 and -5; Myc genes such as C-myc, L-myc and N-myc; Nanog; Lrh genes such as Lhr1, and Rar genes such as Rara or rar-g and Lin28 may also be introduced into the cell to increase induction efficiency. Following introduction of the pluripotency genes or proteins, the ancestor cells may be cultured. Cells expressing pluripotency markers may be isolated and/or purified to produce a population of iPSCs. Techniques for the production of iPSCs are well-known in the art (Yamanaka et al Nature 2007; 448:313-7; Yamanaka 6 2007 Jun. 7; 1(1):39-49; Kim et al Nature. 2008 Jul. 31; 454(7204):646-50; Takahashi Cell. 2007 Nov. 30; 131(5):861-72. Park et al Nature. 2008 Jan. 10; 451(7175):141-6; Kimet et al Cell Stem Cell. 2009 Jun. 5; 4(6):472-6; Vallier, L., et al. Stem Cells, 2009. 9999(999A), Wang W, et al. PNAS. (2011) 108; 45; 18283-8.

5. Tissue-Derived TSCs

The disclosed TSCs can be derived from tissues as demonstrated in the non-limiting examples. Tissue-derived stem cells can be found in tissues such as bone marrow (BM), blood vessels, adipose tissues, and placental and dental pulp. In preferred forms, the disclosed TSCs are derived from perinatal tissues. Perinatal tissues refer to tissues that are discarded at birth, such as the placenta, umbilical cord, cord blood, and amniotic fluid, and different stem and progenitor cell types can be isolated from these tissues.

In some forms, the disclosed TSCs are derived from amniotic fluid and/or amniotic membrane. In some forms, the disclosed TSCs are derived from amniotic fluid. Amniotic fluid contains a heterogeneous cell population according to their morphologies and growth, in vitro biochemical characteristics and in vivo potential. Amniotic fluid mainly includes three types of cells: epithelioid type cells derived from fetal skin and urine, amniotic fluid type derived from the fetal membranes and trophoblast, and fibroblastic type cells derived from fibrous connective tissues and dermal fibroblasts. Based on plastic adherence, two populations of amniotic fluid cells can be isolated: the amniotic fluid mesenchymal stem cells (AFMSC) and the amniotic fluid stromal cells (AFSC). In some forms, the disclosed TSCs are derived from the amniotic membrane. The amniotic membrane is the inner layer of the amniotic sac or extra-embryonic fetal membranes and is composed of three layers: an epithelial monolayer, an acellular basement layer, and a mesenchymal cell layer. In some forms, the disclosed TSCs are derived from the amniotic membrane mesenchymal stromal cells (AMSC). In some forms, the disclosed TSCs are derived from and the amniotic epithelial cells (AEC).

In some forms, the disclosed TSCs are derived from umbilical cord tissues, umbilical cord blood and/or Wharton's jelly. The umbilical cord attaches the embryo to the placenta guaranteeing the continuous supply of nutrients and oxygen to the fetus during pregnancy.

In some forms, the disclosed TSCs are derived from chorionic membranes. The chorionic membrane (CM) is the outer layer of the human extra-embryonic fetal membranes and connects the fetus to the maternal tissues. The CM is in close contact with the decidua and is separated from the amniotic membrane by a spongy layer of collagen fibers. In some forms, the disclosed TSCs are derived from chorionic plate tissues. The chorionic plate is made up of the amino-chorionic membrane and fetal vessels. The stem cells are isolated from the closest region to the umbilical cord once the amniotic membrane is removed and the isolated cells have a mesenchymal type of phenotype and known as chorionic plate MSC (CP-MSC). In some forms, the disclosed TSCs are derived from chorionic villi. Chorionic villi (CV) are finger-like projections that sprout from the chorion, and together with the maternal tissue of the basal plate form the placenta.

In some forms, the disclosed TSCs are derived from maternal decidua and/or placental tissue. The decidua is the maternal component of placental tissues and is divided into three regions: the decidua basalis that originates at the site of embryo implantation, the decidua capsularis that encloses the embryo, and the decidua parietalis that covers the rest of the uterus and fuses with the decidua capsularis by the fourth month of pregnancy.

C. Coronavirus Particles to be Cultured

The disclosed coronavirus medium containing the eSTBs can be used to culture coronavirus particles. In some forms, the coronavirus particles are derived from mammalian or non-mammalian coronaviruses such as for example, human coronaviruses, feline coronaviruses, canine coronaviruses, porcine coronaviruses, bovine coronaviruses, dromedary camel coronaviruses, murine coronaviruses, or variants thereof. In preferred forms, the coronavirus particles to are derived from human coronaviruses. In some forms, the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles, and/or variant particles thereof. In some forms, culture coronavirus particles to be cultured is an alpha coronavirus, beta coronavirus, delta coronavirus, and/or gamma coronavirus, or sub-variants and/or sub-variant particles thereof.

1. Coronaviruses and SARS-CoV-2

The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host specificity and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ˜30% of mild upper respiratory tract illnesses.

At ˜30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which includes approximately two-thirds of the genome, encodes two polyproteins (encoded by ORF1a and ORF1b) that undergo co-translational proteolytic processing. There are four open reading frames (ORFs) downstream of rep that are predicted to encode the structural proteins, S, E, M, and N, which are common to all known coronaviruses.

In some forms, the coronavirus particle is derived from COVID associated with SARS-CoV-2 betacoronavirus of the subgenus Sarbecovirus. SARS-CoV-2 viruses share approximately 79% genome sequence identity with the SARS-CoV virus identified in 2003. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N). In addition, seven putative ORFs encoding accessory proteins are interspersed between the structural genes.

In some forms, the coronavirus particles are derived from a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant). In some forms, the SARS-CoV-2 particles are SARS-CoV-2 alpha variant particles, SARS-CoV-2 beta variant particles, SARS-CoV-2 gamma variant particles, SARS-CoV-2 delta variant particles, SARS-CoV-2 epsilon variant particles, SARS-CoV-2 eta variant particles, SARS-CoV-2 iota variant particles, SARS-CoV-2 kappa variant particles, SARS-CoV-2 mu variant particles, SARS-CoV-2 omicron variant particles, SARS-CoV-2 zeta variant particles, SARS-CoV-2 1.617.3 variant particles and/or SARS-CoV-2 lambda variant particles.

In some forms, the coronavirus particles can be derived from a sub-variant of the SARS-CoV-2 B.1.1.7 (Alpha variant), a sub-variant of the SARS-CoV-2 B.1.351 (Beta variant), a sub-variant of the SARS-CoV-2 P.1 (Gamma variant), a sub-variant of the SARS-CoV-2 B.1.617, a sub-variant of the SARS-CoV-2 B.1.617.1 (Kappa variant), a sub-variant of the SARS-CoV-2 B.1.621 (Mu variant), a sub-variant of the SARS-CoV-2 B.1.617.2 (Delta variant), a sub-variant of the SARS-CoV-2 B.1.617.3, or a sub-variant of the SARS-CoV-2 B.1.1.529 (Omicron variant), or a sub-variant derived from a descendent lineage of one or more of the foregoing sub-variants. For example, when the SARS-CoV-2 variant is an Omicron variant, the Omicron sub-variant can be a BA.1 sub-variant, a BA.2 sub-variant, a BA.3 sub-variant, a BA.4 sub-variant, a BA.5 sub-variant, or a BA.1/BA.2 circulating recombinant sub-variant such as XE.

2. SARS-CoV

In some forms, the coronavirus particle to be cultured is derived from the replication of SARS-CoV and variants thereof. SARS-CoV predisposes the host to developing severe acute respiratory syndrome, otherwise known as SARS. SARS is caused by the SARS coronavirus, known as SARS CoV. SARS CoV is believed to be a strain of the coronavirus usually only found in small mammals that have mutated, thereby enabling it to infect humans.

A wide range of clinical manifestations are seen in patients with SARS from mild, moderate, to severe and rapidly progressive and fulminant disease. The estimated mean incubation period of SARS-CoV infection was 4.6 days (95% CI, 3.8-5.8 days) and 95% of illness onset occurred within 10 days. The mean time from symptom onset to hospitalization was between 2 and 8 days but was shorter toward the later phase of the epidemic. The mean time from symptom onset to need for invasive mechanical ventilation (IMV) and to death was 11 and 23.7 days, respectively.

The major clinical features of SARS are fever, rigor, chills, myalgia, dry cough, malaise, dyspnea, and headache. Sore throat, sputum production, rhinorrhea, nausea, vomiting, and dizziness are less common. Watery diarrhea was present in 40% to 70% of patients with SARS and tended to occur about 1 week after illness onset. SARS-CoV was detected in the serum and cerebrospinal fluid of 2 patients complicated by status epilepticus. Elderly patients with SARS-CoV infection might present with poor appetite, a decrease in general well-being, fracture as a result of fall, and confusion, but some elderly subjects might not be able to mount a febrile response. In contrast, SARS-CoV infection in children aged less than 12 years was generally mild, whereas infection in teenagers resembled that in adults. There was a low mortality rate among young children and teenagers. SARS-CoV infection acquired during pregnancy carried a case fatality rate of 25% and was associated with a high incidence of spontaneous miscarriage, preterm delivery, and intrauterine growth retardation without perinatal SARS-CoV infection among the newborn infants.

Asymptomatic SARS-CoV infection was uncommon in 2003; a meta-analysis had shown overall sero-prevalence rates of 0.1% (95% CI, 0.02-0.18) for the general population and 0.23% for health care workers (95% CI, 0.02-0.45) in comparison with healthy blood donors, others from the general community, or patients without SARS-CoV infection recruited from the health care setting (0.16%, 95% CI, 0-0.37).

The clinical course of patients with SARS-CoV infection seemed to manifest in different stages. In the first week of illness of SARS-CoV infection, many patients presented with fever, dry cough, myalgia, and malaise that might improve despite the presence of lung consolidation and rising viral loads on serial samples. During the second week, many patients experienced recurrence of fever, worsening consolidation, and respiratory failure, while about 20% of patients progressed to ARDS requiring IMV. Peaking of viral load on day 10 of illness corresponded temporally to peaking of the extent of consolidation radiographically, and a maximal risk of nosocomial transmission, particularly to health care workers.

The disclosed compositions containing eSTBs and methods of using thereof may be used to culture SARS-CoV particles extracted from patients infected with SARS-CoV and/or variants thereof, for viral studies and/or antiviral drug evaluation.

3. MERS-CoV

In some forms, the disclosed compositions containing the eSTBs and methods of using thereof, can be used to culture the Middle East respiratory syndrome-related coronavirus (MERS-CoV) and variants thereof. MERS-CoV predisposes the host to developing Middle East Respiratory Syndrome (MERS). MERS-CoV is a coronavirus believed to be originally from bats. However, humans are typically infected from camels, either during direct contact or indirectly. Spread between humans typically requires close contact with an infected person. As of 2021, there is no specific vaccine or treatment for the disease, although attempts are being made.

The virus MERS-CoV is a member of the beta group of coronaviruses, Betacoronavirus, lineage C. MERS-CoV genomes are phylogenetically classified into two clades, clade A and B. The earliest cases were of clade A clusters, while the majority of more recent cases are of the genetically distinct clade B. MERS-CoV is closely related to the Tylonycteris bat coronavirus HKU4 and Pipistrellus bat coronavirus HKU5.

The specific exposures that lead to sporadic MERS-CoV infections are unknown, therefore it is challenging to estimate the incubation period in primary cases. However, based on data from cases of human-to-human transmission, the incubation period is a median of 5-7 days, with a range of 2-14 days (median 5-2 days [95% CI 1.9-14.7]). Immunocompromised patients can present with longer incubation periods of up to 20 days.

4. Common Human Coronaviruses

Unlike the highly pathogenic SARS-CoV, MERS-CoV, and 2019-nCoV, the four so-called common HCoVs generally cause mild upper-respiratory tract illness and contribute to 15%-30% of cases of common colds in human adults, although severe and life-threatening lower respiratory tract infections can sometimes occur in infants, elderly people, or immunocompromised patients. In some forms, disclosed compositions containing eSTBs and methods of using thereof, can be used to culture the HCoVs for viral studies and to screen for drugs to reduce the replication and ameliorate the pathology associated with one or more of the four common HCoVs.

Human coronavirus 229E (HCoV-229E) is a species of coronavirus which infects humans and bats. HCoV-229E is a member of the genus Alphacoronavirus and subgenus Duvinacovirus. It is an enveloped, positive-sense, single-stranded RNA virus which enters its host cell by binding to the APN receptor. HCoV-229E is associated with a range of respiratory symptoms, ranging from the common cold to high-morbidity outcomes such as pneumonia and bronchiolitis. However, such high morbidity outcomes are almost always seen in cases with co-infection with other respiratory pathogens. In some forms, HCoV-229E may cause acute respiratory distress syndrome (ARDS). HCoV-229E is also among the coronaviruses most frequently co-detected with other respiratory viruses, particularly with human respiratory syncytial virus (HRSV).

Human coronavirus NL63 (HCoV-NL63) is a species of coronavirus, specifically a Setracovirus from among the Alphacoronavirus genus. The virus is an enveloped, positive-sense, single-stranded RNA virus which enters its host cell by binding to ACE2. The virus is found primarily in young children, the elderly, and immunocompromised patients with acute respiratory illness. It also has a seasonal association in temperate climates. The evolution of HCoV-NL63 appears to have involved recombination between an ancestral NL63-like virus circulating in African Triaenops afer bats and a CoV 229E-like virus circulating in Hipposideros bats. Recombinant viruses can arise when two viral genomes are present in the same host cell. The first cases of the infection with HCoV-NL63 were found in young children with severe lower respiratory tract infections admitted to hospitals. While the clinical presentation of the virus can be severe, it has also been found in mild cases of respiratory infection. The comorbidity of HCoV-NL63 with other respiratory infections, has made the specific symptoms of the virus difficult to pinpoint. A study of clinical symptoms in HCoV-NL63 patients without secondary infection, reported the most common symptoms to be fever, cough, rhinitis, sore throat, hoarseness, bronchitis, bronchiolitis, pneumonia, and croup. An early study investigating children with lower respiratory tract illness, found that HCoV-NL63 was more commonly found in outpatients than hospitalized patients, suggesting that it is a common cold virus similar to HCoV-229E and HCoV-OC43, which generally cause less severe symptoms.

Human coronavirus OC43 (HCoV-OC43) is a member of the species Betacoronavirus 1, which infects humans and cattle. The infecting coronavirus is an enveloped, positive-sense, single-stranded RNA virus that enters its host cell by binding to the N-acetyl-9-O-acetylneuraminic acid receptor. Four HCoV-OC43 genotypes (A to D) have been identified, with genotype D most likely arising from genetic recombination. The complete genome sequencing of genotypes C and D and bootscan analysis shows recombination events between genotypes B and C in the generation of genotype D. Of 29 viral variants identified, none belong to the more ancient genotype A. Symptoms of an infection with HCoV-OC43 are as described for HCoV-229E and HCoV-NL63.

Human coronavirus HKU1 (HCoV-HKU1) is an enveloped, positive-sense, single-stranded RNA virus which like the OC43 virus, enters its host cell by binding to the N-acetyl-9-O-acetylneuraminic acid receptor. HCoV-HKU1 has the Hemagglutinin esterase (HE) gene, which distinguishes it as a member of the genus Betacoronavirus and subgenus Embecovirus. Symptoms of an infection with HCoV-HKU1 are as described for HCoV-229E and HCoV-NL63.

5. Non-Human Coronaviruses

The disclosed compositions containing the eSTBs and methods of using thereof, can be used to culture an alpha coronavirus or beta coronavirus that can infect a non-human mammal. In some forms, the alpha coronavirus can be canine enteric coronavirus (CECoV), feline coronavirus (FCoV), porcine respiratory coronavirus (PRCV), porcine epidemic diarrhea virus (PEDV), or transmissible gastroenteritis virus (TGEV). In some forms, the alpha coronavirus can be a variant derived from rhinolophus bat coronavirus HKU2 (Bat-CoV HKU2) or miniopterus bat coronavirus HKU8 (Bat-CoV HKU8). In some forms, the beta coronavirus can be canine respiratory coronavirus (CRCoV), murine coronavirus (M-CoV), porcine hemagglutinating encephalomyelitis virus (PHEV), hedgehog coronavirus 1, bovine coronavirus (B-CoV), porcine enteric coronavirus (PEC), transmissible gastroenteritis virus (TGEV), swine acute diarrhea syndrome coronavirus (SADS-CoV), porcine delta coronavirus (PDCoV), porcine epidemic diarrhea virus (PEDV), or equine coronavirus (E-CoV). In some forms, the beta coronavirus can be a variant derived from tylonycteris bat coronavirus HKU4 (Bat-CoV HKU4), pipistrellus bat coronavirus HKU5 (Bat-CoV HKU5), or rousettus bat coronavirus HKU9 (Bat-CoV HKU9).

The coronavirus particles to be cultured may also be caused by a gamma coronavirus or a delta coronavirus. In some forms, the gamma coronavirus can be Avian Infectious Bronchitis (AIBV) or Beluga Whale CoV SW1. In some forms, the delta coronavirus can be Bulbul CoV HKU11 (BuCoV HKU11), Thrush CoV HKU12 (ThCoV HKU12), Munia CoV HKU13 (MunCoV HKU13), Porcine CoV HKU15 (PDCoV HKU15), White-eye CoV HKU16 (WECoV HKU16), Sparrow CoV HKU17 (SpCoV HKU17), Magpie Robin CoV HKU18 (MRCoV HKU18), Night heron CoV HKU19 (NHCoV HKU19), wigeon CoV HKU20 (WiCoV HKU20), Common moorhen CoV HKU21 (CMCoV HKU21), falcon CoV HKU27 (FalCoV UAE-HKU27), houbara bustard CoV HKU28 (HouCoV UAE-HKU28), pigeon CoV HKU29 (PiCoV UAE-HKU29), and quail CoV HKU30 (QuaCoV UAE-HKU30), which are the best characterized DCoV species. Delta coronaviruses are described in further detail in Vlasova et al. (2021) Frontiers in Veterinary Science, Vol. 10, doi: 10.3389/fvets.2020.626785. Non-human coronaviruses are described in further detail in Kenney et al. 2020 Veterinary Pathology, Vol. 58, Issue 3, pages 438-452, doi: 10.1177/0300985820980842; Alluwaimi et al. (2020) Frontiers in Veterinary Science, Vol. 7, Article number 582287, doi: 10.3389/fvets.2020.582287).

Porcine enteric coronaviruses (PECs), including the transmissible gastroenteritis virus (TGEV), the novel emerging swine acute diarrhea syndrome coronavirus (SADS-CoV), porcine delta coronavirus (PDCoV), and re-emerging porcine epidemic diarrhea virus (PEDV), which infect pigs of different ages, have caused more frequent occurrences of diarrhea, vomiting, and dehydration with high morbidity and mortality in piglets.

D. Kits

The materials described above as well as other materials can be packaged together in any suitable combination as a kit useful for performing, or aiding in the performance of, the disclosed method. It is useful if the kit components in a given kit are designed and adapted for use together in the disclosed method.

In some forms, the kit may be a cell culture kit. In some forms, the cell culture kit may include one or more of TSC cells, culture reagents, and materials suitable for culturing the TSCs and/or their derivatives, SBTs and EVTs.

In some forms, the kit may include one or more tissue precursor cells, tissue stem cells, cells differentiated from ES cells, and/or cells differentiated from iPS cells, culture reagents, and materials suitable for deriving the TSCs and their derivatives, SBTs and EVTs. In some forms, the kits may contain EPSCs, naïve stem cells, primed stem cells, embryonic cells, placental cells, cell culture reagents and/or materials for deriving the disclosed TSCs. Generally, TSCs, regardless of the method of derivation, are capable of generating STBs and EVTs.

In an exemplary form, the kit includes cells, reagents and/or materials for deriving the TSCs and/or their derivatives from EPSCs. In an exemplary form, the kit may include a combination of two or more of:
(i) the fourth culture medium for deriving the EPSCs described herein, comprising one or more of a RAS-ERK inhibitor, a SRC kinase inhibitor, a GSK3 inhibitor, and/or a Tankyras/WNT inhibitor;
(ii) the EPSC maintenance medium comprising one or more of a Ras-ERK inhibitor, a SRC kinase inhibitor, a GSK3 inhibitor and/or a Tankyrase/WNT inhibitor; (iii) the dissociating reagent for producing single EPSCs comprising a trypsin replacement agent;
(iv) the trophoblast stem cell medium comprising basal medium supplemented with one or more of a reducing agent, fetal bovine serum (FBS), an antibiotic, Bovine Serum Albumin (BSA), Epidermal Growth Factor (EGF), Glycogen synthase kinase 3 (GSK-3) inhibitor, an ALK-5 inhibitor, a ROCK inhibitor, a TGF-β inhibitor, and/or an HDAC inhibitor, wherein basal medium is DMEM/F-12 or DMEM;
(v) STB medium comprising basal medium supplemented with one or more of a reducing agent, BSA, an antibiotic, a ROCK inhibitor, a cAMP inhibitor, KSR medium, and/or one or more differentiation agents; and
(vi) coronavirus medium comprising basal medium, wherein basal medium is DMEM/F-12 or DMEM. Kits containing one or more culture media for deriving the TSCs and/or their derivatives, eSTBs and/or EVTs as described herein are also disclosed.

In a second exemplary form, the kit includes cells, reagents and/or materials for deriving the TSCs and/or their derivatives from primed and/or naïve stem cells. In an exemplary form, the kit may include a combination of two or more of:
(i) an Activin/TGF-beta Pathway Inhibitor e.g., A83-01;
(ii) a BMP/TGF-beta pathway inhibitor e.g., SB431542;
(iii) co-factors/growth enzymes e.g., L-ascorbic acid; and/or
(iv) an HDAC inhibitor e.g., valproic acid. In some forms, the kit includes cells, reagents and/or materials for deriving the TSCs and/or their derivatives from naïve PSCs under 3i conditions, 2i/LIF conditions, alternative 2i conditions, and/or LIF/MEKi/aPKCi as described in Section III (B)(4).

In some forms, the kit may include a combination of two or more of the following for deriving the TSCs and/or their derivatives from primed PSCs: (i) glycogen synthase kinase 3 (GSK3) pathway inhibitor, (ii) a small molecule Tankyrase inhibitor, e.g., IWR1, and/or fibroblast growth factor 2 (FGF2).

In some forms, the one or more culture media in the kit may be formulated in deionized, distilled water. In some forms, the one or more culture media will typically be sterilized prior to use to prevent contamination, e.g., by ultraviolet light, heating, irradiation, or filtration. In some forms, the one or more culture media may be frozen (for example, at −20° C. or −80° C.) for storage or transport. In some forms, the one or more culture media may contain one or more antibiotics to prevent contamination.

In some forms, the one or more culture media may be a 1×formulation or a more concentrated formulation, for example, a 2× to 250× concentrated medium formulation. In a 1× formulation each ingredient in the medium is at the concentration intended for cell culture, for example a concentration set out above. In a concentrated formulation one or more of the ingredients is present at a higher concentration than intended for cell culture. Concentrated culture media are well known in the art, such as salt precipitation or selective filtration. A concentrated medium may be diluted for use with water (in certain forms, deionized and distilled) or any appropriate solution, for example, an aqueous saline solution, an aqueous buffer, or a culture medium.

The one or more media in the kit may be contained in hermetically sealed vessels which prevent contamination. Hermetically sealed vessels may be preferred for transport or storage of the culture media. The vessel may be any suitable vessel, such as a flask, a plate, a bottle, a jar, a vial, or a bag.

The kit may also include instructions for use, e.g., for using the TSC media for deriving TSC and/or for using the STB medium to derive eSTBs.

### III. Methods of Making

Methods of inducing trophoblast stem cells (TSCs) and their derivatives are described. The methods engineer TSCs, eSTBs, and/or EVTs having TSC-, eSTB-, or EVT-specific morphologies and/or expressing TSC, eSTB, or EVT-specific molecular signatures.

In some forms, the methods require incubating the pluripotent cells in cell culture media compositions containing combinations of chemical compounds to induce the chemical differentiation of pluripotent cells partially or completely into trophoblast stem cells (TSCs), early syncytiotrophoblasts (eSTBs), and extravillous trophoblasts (EVTs). In some forms, the disclosed TSCs and their derivative eSTBs and EVTs can be generated from human pre-implantation embryos such as for example the blastocyst. In some forms, the disclosed TSCs and their derivative eSTBs and EVTs can be generated from naïve stem cells. In some forms, the disclosed TSCs and their derivative eSTBs and EVTs can be generated from primed stem cells. In a third preferred form, the disclosed TSCs and their derivative eSTBs and EVTs can be generated from first trimester placental trophoblasts. In a fourth preferred form, the disclosed TSCs and their derivative eSTBs and EVTs can be generated from somatic cells via induced pluripotent stem cells. The required combinations of small molecules/protein can vary depending on the stage of trophoblast differentiation.

A. Methods for Generating Trophoblasts

Cell culture media compositions containing combinations of chemical compounds which can be used to induce the chemical differentiation of trophoblast stem cells (TSCs) into early syncytiotrophoblasts (eSTBs), and extravillous trophoblasts (EVTs) are disclosed. Also provided are cell culture media compositions containing combinations of chemical compounds which can be used to derive and/or extract TSCs from various sources. The required combinations of small molecules/protein can vary depending on the stage of trophoblast differentiation. More detailed methods and reagents for EPSC and trophoblast differentiation are described in WO 2016/079146, WO 2020/200071, U.S. Pat. No. 10,745,670, and U.S. Patent Application Publication Nos. 2021/0230556 and 2022/0145264, which are all herein incorporated by reference in their entireties.

The cell culture media includes basal medium supplemented with small molecule factors/proteins as disclosed herein. As used herein, “basal medium” refers to a mixture of salts that provide cells with water and certain bulk inorganic ions essential for normal cell metabolism, maintain intra- and extra-cellular osmotic balance, provide a carbohydrate as an energy source, and provide a buffering system to maintain the medium within the physiological pH range. Basal medias are known in the art, commercially available, and are described in for example WO 2016/079146, WO 2020/200071, U.S. Pat. No. 10,745,670, and U.S. Patent Application Publication Nos. 2021/0230556 and 2022/0145264, which are all herein incorporated by reference in their entireties. Examples of basal medias include, but are not limited to, Dulbecco's Modified Eagle's Medium (DMEM), Minimal Essential Medium (MEM), Basal Medium Eagle (BME), RPMI 1640, Ham's F-10, Ham's F-12, alpha-Minimal Essential Medium (aMEM), Glasgow's Minimal Essential Medium (O-MEM), and Iscove's Modified Dulbecco's Medium (IMDM), STEM PRO®, STEM PRO-34® and mixtures thereof. Preferably, the basal medium is Dulbecco's Modified Eagle's Medium (DMEM), Knock-out DMEM or Dulbecco's Modified Eagle Medium Nutrient Mixture F-12 (DMEM/F-12).

1. Derivation of Trophoblasts

Methods for deriving synctiotrophoblasts (STBs) and extravillous cytotrophoblasts (EVTs) from the TSCs are provided. Typically, the STBs are early or immature STBs i.e., Day 2 (D2) STBs. It has been established that early STBs are only transiently present in hTSC differentiation. The discovery of eSTBs was made possible by the stem cell-based in vitro system since eSTB-like cells also likely only transiently exist in the placenta trophoblast development before they differentiate to the non-proliferative multinucleated STBs. Preferably, eSTBs produced by the disclosed methods have significantly longer survival rates compared to existing cells (e.g., Vero E6 cells) following viral infection as mechanistically confirmed by morphological and molecular assessments provided in the non-limiting examples.

2. Derivation of eSTBs from TSCs

The induction of human trophoblast stem cells to form eSTBs requires culturing the TSCs in a STB medium. Typically, the STB medium contains basal medium supplemented with one or more of (i) a reducing agent, (ii) BSA, (iii) an antibiotic, (iv) a ROCK inhibitor, (v) a cAMP agonist, (vi) KSR medium, and/or (vii) one or more differentiation agents in effective amounts to induce TSCs to form eSTBs. In preferred forms, the basal medium is DMEM/F-12 or DMEM.

In some forms, the STB medium contains a reducing agent to prevent the accumulation of toxic levels of oxygen radicals. In preferred forms, the reducing agent is β-mercaptoethanol also known as 2-mercaptoethanol, BME, 2BME, 2-ME, or β-met. In some forms, reducing agent is present in the STB medium in a concentration of about 20 μM, about 30 μM, about 40 μM, about 50 μM, about 60 μM, about 70 μM, about 80 μM. Preferably, the reducing agent is present in the STB medium in a concentration of about 50 μM.

In some forms, the STB medium contains one or more serum supplements. In some forms, the serum supplement is bovine serum albumin (BSA) in effective amounts. BSA contains growth factors (GFs) and other elements essential for ASC attachment, expansion, maintenance, and proliferation in vitro. In some forms, the STB medium contains BSA at between about 0.1 weight percent, about 0.1 weight percent, about 0.2 weight percent, about 0.3 weight percent, about 0.4 weight percent, about 0.5 weight percent, about 0.6 weight percent, about 0.7 weight percent, about 0.8, about 0.9 weight percent, or about 1.0 weight percent BSA. Preferably, the STB medium contains about 0.3 weight percent BSA. In some forms, the serum supplement is Knock-out Serum Replacement (KSR) medium in effective amounts to enhance cell proliferation. In some forms, the STB medium contains KSR medium at between about 1 weight percent, about 2 weight percent, about 3 weight percent, about 4 weight percent, about 5 weight percent or about 6 weight percent KSR medium. Preferably, the STB medium contains about 4 weight percent KSR medium.

In some forms, the STB medium contains an antibiotic to minimize the loss of valuable cells, reagents, time, and efforts due to contamination. Examples of antibiotics that can be used in the STB medium include but are not limited to penicillin-streptomycin (pen-strep) and gentamicin. In preferred forms, the antibiotic is Penicillin-Streptomycin-Glutamine. In some forms, the antibiotic is present in the STB medium at about 0.20 weight percent, about 0.30 weight percent, about 0.40 weight percent, about 0.50 weight percent, about 0.60 weight percent, about 0.70 weight percent, or about 0.80 weight percent. Preferably, the STB medium contains the antibiotic at about 0.5 weight percent.

In some forms, the STB medium contains a Rho Kinase (ROCK) inhibitor to facilitate maintenance of stem cell phenotypes and stem cell survival when they are dissociated to single cells by preventing dissociation-induced apoptosis. Non-limiting examples of ROCK inhibitors include Y-27632, KD-025, thiazovivin, Y39983, AT13148, and LX-7101. In preferred forms, the ROCK inhibitor present in the STB medium is Y-27632. In some forms the STB medium contains the ROCK inhibitor in a concentration of about 1.5 μM, about 2.0 μM, about 2.5 μM, about 3.0 μM or about 3.5 μM, preferably about at about 2.5 μM.

In some forms, the STB medium contains molecules that improve or boost differentiation of TSCs. Preferably, the molecules that improve or boost differentiation are cyclic AMP (cAMP) agonists. Non-limiting examples of cyclic AMP agonists include prostaglandin E2 (PGE2), dibutyl cyclic-AMP (dbcAMP), 8-Br-cAMP, genistein, Forskolin (FSK), colforsin, and rolipram. Preferably, the cyclic AMP agonist used to improve differentiation of TSCs to eSTBs is forskolin. A preferred cAMP agonist is Forskolin used in a concentration ranging from about 1.0 μM, about 2.0 μM, about 3.0 μM, about 4.0 μM, about 5.0 μM, about 6.0 μM, about 7.0 μM, about 8.0 μM, about 9.0 μM or about 10 μM. Preferably, the STB medium contains the cAMP agonist in a concentration of about 2.0 μM.

In some forms, the STB medium contains one or more differentiation agents and/or growth factors. “Differentiation factors” as used herein are factors which modulate, for example promote or inhibit, a signaling pathway which mediates differentiation in a mammalian cell. Differentiation factors may include growth factors, cytokines and inhibitors. In preferred forms, the differentiation agent included in the STB serum is Insulin-Transferrin-Selenium-Ethanolamine (ITS-X) at between about 0.5 weight percent to about 2 weight percent, preferably 1 weight percent ITS-X. Insulin promotes glucose and amino acid uptake, lipogenesis, intracellular transport, and the synthesis of proteins and nucleic acids. Transferrin is an iron carrier and may also help to reduce toxic levels of oxygen radicals and peroxide. Selenium, as sodium selenite is a co-factor for glutathione peroxidase and other proteins that is used as an antioxidant in media. Ethanolamine is a precursor of phospho-glycerides which are essential to the structure of the plasma membrane and cellular organelles. In some forms, the differentiation agent includes one or more of a cAMP analog and/or a PKA activating agent, preferably 8-Bromoadenosine 3′,5′-cyclic monophosphate sodium salt (8-Bromo-cAMP sodium salt). 8-Bromo-cAMP sodium salt is a cell-permeable cAMP analog that has greater resistance to hydrolysis by phosphodiesterase than cAMP, activates protein kinase A, inhibits growth, decreases proliferation, increases differentiation, and/or induces apoptosis of cultured cells.

In an exemplary form, the STB medium contains DMEM/F12 (ThermoFisher Scientific, Catalog #21331020), supplemented with 50 μM β-mercaptoethanol, 0.5% Penicillin-Streptomycin-Glutamine (ThermoFisher Scientific, Catalog #10378016), 0.3% BSA, 1% ITS-X, 2.5 μM Y-27632, 2 μM Forskolin (Sigma-Aldrich, Catalog #F3917), and 4% KnockOut Serum Replacement (ThermoFisher Scientific, Catalog #10828028).

In a preferred form, TSCs are induced to generate eSTBs via the following steps:
(i) coating the wells of a six-well plate with 100×Matrigel (Corning, Catalog #354230) for at least 1 hour;
(ii) seeding about 1.0×10⁵ TSCs per well in about 2 mL STB medium; and
(iii) changing the media on day 3, with the cells being ready for downstream analysis on day 6.

Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques' by A. Doyle and J. B. Griffiths (1997), ‘Human Embryonic Stem Cells’ by A. Chiu and M. Rao (2003), Stem Cells: From Bench to Bedside’ by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 370 C, 21% Oxygen, 5% Carbon Dioxide.

B. Derivation of TSCs

The disclosed trophoblast stem cells (TSCs) can be derived from various sources including but not limited to embryonic tissues, fetal tissues, adult tissues and differentiated somatic cells after they have been genetically reprogrammed (referred to as induced pluripotent stem cells (iPSCs)). In some forms, the disclosed TSCs can be derived from expanded potential stem cells (EPSCs). In some forms, the disclosed TSCs can be derived from naïve stem cells. In some forms, the disclosed TSCs can be derived from primed stem cells. In some forms, the disclosed TSCs can be derived from placental or blastocyst cells.

1. Derivation of TSCs from EPSCs

The disclosed trophoblast stem cells (TSCs) can be derived from expanded potential stem cells (EPSCs). In some forms, the induction of trophoblast lineages can be accomplished by culturing the EPSCs in two cell culture media for varying amounts of time. In some forms, the trophoblast lineage cells are derived by:
(i) culturing dissociated EPSCs for about 24 hours in a second culture medium containing one or more of knock-out serum replacement (KSR) medium, growth factors, and a ROCK inhibitor; and
(ii) culturing the hEPSCs from step (i) in a third culture medium containing a TGF-β inhibitor and KSR medium.

In some forms, the induction of expanded potential stem cells (EPSCs) to form TSCs is accomplished by culturing the EPSCs in TSC medium. In these forms, the TSCs can be derived by culturing the EPSCs from Step III (A)(1) in a culture medium containing trophoblast stem cell medium, thereby producing TSC colonies, the details of which are described in non-limiting Example 1.

Methods and reagents for the derivation of TSCs are described in WO 2016/079146, WO 2020/200071, U.S. Pat. No. 10,745,670, U.S. Patent Application Publication Nos. 2021/0230556 and 2022/0145264, and Okae, H. et al. (2018) Cell Stem Cell 22(1), pages 50-63.e6, which are incorporated herein by reference in their entireties. In a preferred form, the TSCs are derived by plating single cell-dissociated EPSCs on 6-well plates pre-coated with 100×Geltrex (for example, ThermoFisher Scientific, Catalog #A1413302) at a density of about 2,000 cells per well and culturing in a first culture medium containing Trophoblast Stem Cell (TSC) media. In preferred forms, the TSC media is human TSC (hTSC) media.

Typically, the TSC media contains a basal medium, preferably DMEM/F-12 or DMEM supplemented with one or more of (i) a reducing agent, (ii) fetal bovine serum (FBS), (iii) an antibiotic, (iv) Bovine Serum Albumin (BSA), (v) Epidermal Growth Factor (EGF), (vi) Glycogen synthase kinase 3 (GSK-3) inhibitor, (vii) an ALK-5 inhibitor, (viii) a ROCK inhibitor, (ix) a TGF-β inhibitor, and/or an (x) HDAC inhibitor.

In some forms, the TSC media contains the reducing agent β-mercaptoethanol in a concentration of about 30.0 μM to about 70.0 μM, preferably about 50.0 μM.

In some forms, the TSC media contains basal medium supplemented with about 1% FBS to about 20% FBS, preferably about 0.2% FBS.

In some forms, the TSC media contains the antibiotic penicillin-Streptomycin-Glutamine at a weight percent of about 1%, 2%, 3%, 4%, 5%, preferably about 1%.

In some forms, the Epidermal Growth Factor is present in the trophoblast stem cell medium at a concentration of about 50.0 ng/Ml.

In some forms, the TSC media contains the GSK-3 inhibitor CHIR99021 in a concentration of about 2.0 μM.

In some forms, the TSC media contains the ALK-5 inhibitor A83-01 in a concentration of about 0.5 μM.

In some forms, the TSC media contains the TGF-β inhibitor is SB431542 in a concentration of about 1.0 uM.

In some forms, the TSC media contains the HDAC inhibitor valproic acid at a concentration of about 10.0 μM.

In an exemplary form, the TSC media contains DMEM/F12 (for example Gibco, Catalog #21331-020) supplemented with about 50.0 μM β-mercaptoethanol (ThermoFisher Scientific, Catalog #31350010), about 0.2% FBS (Gibco, Catalog #10270), about 0.5% Penicillin-Streptomycin-Glutamine (ThermoFisher Scientific, Catalog #10378016), about 0.3% BSA (Bovine Serum Albumin, Gibco, Catalog #15260037), 1.0% ITS-X supplement (Gibco, Catalog #51500056), about 50.0 μg/mL 2-Phospho-L-ascorbic acid trisodium salt (a Vitamin C derivative, Sigma-Aldrich, Catalog #49752-100G), about 50.0 ng/mL Epidermal Growth Factor (EGF) (ThermoFisher Scientific, #PHG0311), about 2.0 μM CHIR99021 (Glycogen synthase kinase 3 (GSK-3) inhibitor, Tocris Bioscience, Catalog #4423), about 0.5 μM A83-01 (ALK-5 inhibitor, Tocris Bioscience, Catalog #2939), about 1.0 μM SB431542 (Tocris Bioscience, Catalog #1614), about 10.0 μM Valproic acid (VPA, StemCell Technologies, Catalog #72292) and about 5.0 μM Y27632 (Tocris Bioscience, Catalog #1254). Exemplary methods for deriving the TSCs from EPSCs are described in Okae et al (2018) Cell Stem Cell 22(1), pages 50-63.e6) with the modifications described above.

In a preferred form, TSCs are cultured in the TSC media for about 12 to 14 days after which the colonies with TSC-like morphologies are selected, dissociated in TrypLE (for example Gibco, Catalog #12605036), and re-plated on a plate pre-coated with 100×Geltrex.

In some forms, the cells are passaged for about 4 to about 5 passages, then collected for differentiation of syncytiotrophoblasts (STB) and extravillous trophoblasts (EVT).

2. Derivation of TSCs from Pre-Implantation Embryos

The disclosed trophoblast stem cells (TSCs) can be derived from pre-implantation embryos, e.g., blastocyst cells. In some forms, the pre-implantation embryos are cryopreserved embryos. In some forms, the peri-implantation embryos are fresh embryos obtained via intracytoplasmic sperm injection (ICSI). In some forms, the embryos are collected by (1) culturing the embryos in G1 medium (Vitrolife), (2) covering the embryos with Ovoil™ (Vitrolife) for about 3 days (from the fertilized oocyte to about the 8-cell stage), (3) replacing the GI medium with G2 medium until the blastocyst stage, and (4) selecting the embryos with normal morphology and cleavage patterns.

In an exemplary form, the embryos are cultured for the collection of single cells via one by (1) treating zona-containing blastocysts with acidic Tyrode's solution (e.g., Sigma), (2) pipetting to remove the zona (3) collecting the zona free embryos in in vitro culture medium 1 (IVC1 medium), (4) incubating the embryos in the IVC1 medium for a period of time e.g. for about 7 to 8 days, (5) replacing the IVC1 medium with IVC2 medium, (6) exchanging the IVC2 medium for fresh IVC2 medium about every 24 hours, (7) recovering the embryos from the plate at about day 14, (8) transferring the embryos to a digestion medium (e.g. accutase medium containing 0.25% trypsin, 1:1 ratio) at 37° C. for about 15 minutes to about 30 minutes, (9) physically digesting the embryos to isolate single cells, pipetting the embryos with glass pipettes, (10) incubating the single cells in cold phosphate buffered saline, and (11) picking the individual cells into a lysis medium on ice.

3. Derivation of TSCs from Placental Cells

The disclosed trophoblast stem cells (TSCs) can be extracted from placental tissues. In preferred forms, the TSCs are extracted from first trimester placental cells, i.e., about 6-9 weeks following gestation. In an exemplary form, the TSCs (CT cells) are extracted from placental cells by: (1) cutting whole placental villi into small pieces and enzymatically digesting them three times in a solution containing cell culture dissociation reagents for a period of time at a specified temperature e.g., TrypLE and Accumax for 20 minutes at 37° C.; (2) filtering the cell suspensions; (3) purifying the TSCs using a cell isolation kit; and optionally (4) evaluating cell purity e.g., via flow cytometry. An exemplary method of extracting TSCs from placental tissues is described in Okae, H. et al. (2018) Cell Stem Cell 22(1), pages 50-63.e6).

4. Derivation of TSCs from Naïve and Primed Stem Cells

The disclosed TSCs can be derived from primed pluripotent stem cells (PSCs). In some forms, the TSCs may be derived from primed PSCs under FGF2/Activin A conditions, which include defined primed pluripotency growth conditions for epiblast stem cells, composed of recombinant fibroblast growth factor 2 (FGF2) and Activin A cytokines. In some forms, the TSCs may be derived from primed PSCs under GSK3i/IWR1 conditions, which include defined primed pluripotency growth conditions for epiblast stem cells, containing a glycogen synthase kinase 3 (GSK3) pathway inhibitor and the small-molecule Tankyrase inhibitor, IWR1. In some forms, the TSCs may be derived from primed PSCs under FGF2/IWR1 conditions, which include defined primed pluripotency growth conditions for epiblast stem cells, containing recombinant fibroblast growth factor 2 (FGF2) and the small-molecule Tankyrase inhibitor, IWR1.

In exemplary methods, the TSCs can be derived from primed PSCs by: (1) culturing conventional primed PSCs, e.g., primed human PSCs in basal medium supplemented with a TGF-β agonist, an FGF agonist, and a WNT pathway inhibitor, (2) passaging the cells using a passaging reagent every 4 to 6 days; and (3) culturing primed PSCs in 5% CO2 and 20% 02. In some forms the TGF-β agonist is Activin A in a concentration of about 4 ng/mL to about 6 ng/mL. In some forms, the FGF agonist is FGF2 in a concentration of about 4 ng/mL to about 6 ng/mL. In some forms, the WNT pathway inhibitor is XAV in a concentration of about 1 ng/mL to about 3 ng/mL.

Differentiation of primed PSCs can be accomplished by (1) plating primed PSCs in differentiation medium on Geltrex at about 1:6 to 1:10 ratio and (2) exchanging assay conditions by changing medium every day until assaying. Exemplary culture conditions are described in Table 3. In some forms, the differentiation medium is AFX medium containing a basal medium (e.g., N2B27), one or more growth factors (e.g., Activin A and/or FGF2), and a WNT inhibitor (e.g., XAV medium). Exemplary methods for deriving TSCs from primed PSCs are described in Dong, C. et al. (2020) Elife Vol. 9, Article e52504 and Wei, Y. et al. (2021) Science Advances Vol. 7, article eabf4416.

The disclosed TSCs can be derived from naïve pluripotent stem cells (PSCs). In some forms, the TSCs may be derived from naïve PSCs under 3i conditions, which include defined naïve pluripotency growth conditions combining three inhibitors (i) for MEK, (ii) fibroblast growth factor (FGF) and (iii) glycogen synthase kinase 3 (GSK3) signaling. In some forms, the TSCs may be derived from naïve PSCs under 2i/LIF conditions, which include defined naïve pluripotency growth conditions containing two inhibitors (i) for MEK and (ii) GSK3, together with LIF cytokine. In some forms, the TSCs may be derived from naïve PSCs under alternative 2i conditions, which include defined naïve pluripotency growth conditions containing two inhibitors (i) for the glycogen synthase kinase 3 (GSK3) and (ii) SRC pathways. In some forms, the TSCs may be derived from naïve PSCs under LIF/MEKi/aPKCi conditions, which include defined naïve pluripotency growth conditions containing two inhibitors (i) for MEK and (ii) atypical protein kinase C (aPKC) signaling, together with the leukemia inhibitory factor (LIF) cytokine.

In exemplary methods, the TSCs can be derived from naïve PSCs by: (1) dissociating a population of naïve PSCs; (2) serially culturing the single cell-dissociated naïve stem cells in various culture media and passaging them for a period of time; and (3) inducing the TSCs in TSC medium after a period of time (about 3 days). Exemplary culture conditions are described in Table 3. In some forms, the culture medium contains one or more MEK inhibitors, growth factors, and ALK inhibitors. In some forms the MEK inhibitor is PD0325901 in a concentration of about 1 μM. In some forms, the ALK inhibitor is A83-01 in a concentration of about 1 μM. Exemplary methods for deriving TSCs from naïve PSCs are described in Dong, C. et al. (2020) Elife Vol. 9, Article e52504 and Guo, G. et al. (2021). Cell Stem Cell 28(6), pages 1040-1056. E6.

C. Production of EPSCs from Pluripotent Stem Cells

The production of EPSCs typically includes one or more of the following steps:
(a) isolating pluripotent stem cells for example embryonic stem cells (ESCs) or induced pluripotent stem cells (iPSCs);
(b) incubating the pluripotent stem cells in an EPSC maintenance (EPSCM) medium for a time period sufficient to generate EPSCs;
(c) optionally, maintaining the derived EPSCs in an EPSC maintenance medium; and
(d) optionally, reprogramming somatic cells to acquire a pluripotent state, and/or culturing the reprogrammed cell colonies directly in EPSC medium, bypassing the conventional stages typically required for iPSC cell reprogramming.

Typically, the culture media used to incubate the pluripotent stem cells of step (b) contains one or more of (i) a RAS-ERK inhibitor, (ii) a SRC kinase inhibitor, (iii) a GSK3 inhibitor, and (iv) a WNT inhibitor. Methods for the derivation of EPSCs are known in the art and are described in for example, WO 2016/079146, WO 2020/200071, U.S. Pat. No. 10,745,670, and U.S. Patent Application Publication Nos. 2021/0230556 and 2022/0145264, which are all herein incorporated by reference in their entireties.

In some forms, the EPSCM includes a RAS-ERK inhibitor to facilitate derivation and maintenance of EPSC cells. Many RAS-ERK inhibitors are known in the art and are commercially available. RAS-ERK inhibitors are known in the art and are described in for example, WO 2016/079146, WO 2020/200071, U.S. Pat. No. 10,745,670, and U.S. Patent Application Publication Nos. 2021/0230556 and 2022/0145264, which are all herein incorporated by reference in their entireties. Non-limiting examples of RAS-ERK inhibitors include MEK inhibitors such as PD0325901 (Sigma-Aldrich PZ0162-PD 0325901), a selective, cell permeable MEK1/2 inhibitor that inhibits the activation and downstream signaling of MEK (MEK1 and MEK2). Other MEK inhibitors include PD 334581, PD 198306, PD 184352, Arctigenin, BIX 02189, PD 98059 which are commercially available from Tocris Bioscience. Ras inhibitors include Farnesyl Thiosalicylic Acid (catalogue number sc-221800) and FPT Inhibitor II (catalogue number sc-221626), Manumycin A (sc-200857), L-744,832 Dihydrochloride (sc-221800), FTI-276 trifluoroacetate salt (sc-215057) available from ChemCruz Biochemicals.

The SRC family kinases (SFK) are a family of non-receptor tyrosine kinases that included nine highly related members. Broad spectrum SRC Kinase family inhibitors which inhibit multiple SRC family members are available and known in the art, and are described in for example, WO 2016/079146, WO 2020/200071, U.S. Pat. No. 10,745,670, and U.S. Patent Application Publication Nos. 2021/0230556 and 2022/0145264, which are all herein incorporated by reference in their entireties. Suitable SRC Kinase family inhibitors include A-419259 which is a broad spectrum SRC family kinase inhibitor. Other suitable SRK inhibitors include PP1, PP2 and CGP77675 also available from Sigma-Aldrich, and A419259 trihydrochloride or KB SRC 4 available from Tochris Bioscience. In some forms, the SRC kinase inhibitor is A-419259, XAV939, a Tankyrase inhibitor, or a combination thereof.

In some forms, the EPSC maintenance medium contains one or more GSK3 selective inhibitors such as CHIR99021 (STEMOLECULE™ CHIR99021 Stemgent), 6-Bromolndirubin-3′-Oxime (BIO) (Cayman Chemical (cat:13123)), or STEMOLECULE™ BIO from Stemgent (cat:04003). The GSK3 selective inhibitors contemplated are for example selective inhibitors for GSK-3a/p. GSK3 inhibitors are known in the art, are commercially available, and are described in for example, WO 2016/079146, WO 2020/200071, U.S. Pat. No. 10,745,670, and U.S. Patent Application Publication Nos. 2021/0230556 and 2022/0145264, which are all herein incorporated by reference in their entireties. Suitable GSK3 inhibitors include CHIR99021, a selective and potent GSK3 inhibitor available from Tocris Bioscience (cat 4423), or BIO (cat 3194), A 1070722 (cat 4431), 3F8 (cat 4083), AR-A 014418 (cat 3966), L803-mts (cat 2256) and SB 216763 (cat 1616) also available from Tocris Bioscience. Other suitable GSK inhibitors include GSK-3 Inhibitor IX (available from Santa Cruz Biotechnology sc-202634). Preferably, the GSK-3 inhibitor is CHIR99021.

In some forms, the EPSC maintenance medium contains one or more WNT and/or Tankyrase inhibitors. Preferably the WNT inhibitor inhibits ubiquitination of the β-catenin destruction complex, which stabilizes the destruction complex and prevents β-catenin accumulation, thereby inhibiting WNT signaling. Suitable WNT inhibitors include for example IWP2 (N-(6-Methyl-2-benzothiazolyl)-2-[(3,4,6,7-tetrahydro-4-oxo-3-phenylthieno[3,2-d]pyrimidin-2-yl)thio]-acetamide; Sigma); Dickkopf-related protein 1 (DKK1; R & D Systems), WNT-C59 (4-(2-Methyl-4-pyridinyl)-N-[4-(3-pyridinyl)phenyl]benzeneacetamide) and/or XAV939 (3,5,7,8-Tetrahydro-2-[4-(trifluoromethyl)phenyl]-4H-thiopyrano[4,3-d]pyrimidin-4-one; Sigma), and IWR-1 (molecular formula: C₂₅H₁₉N₃O₃). A preferred WNT inhibitor is IWP2 used in a concentration ranging from 0.5 μM and about 4 μM, preferably 2 μM.

In some forms, the EPSCM may contain one or more notch and/or integrin and/or hippo pathway activators. In some forms, the EPSCM may contain a combination or inhibitors and activators. Notch inhibitors include those which target (or inhibit) gamma-secretase which cleaves Notch intracellular domain, which in turn acts as a transcription co-activator. Thus, in some forms the notch inhibitor is a gamma-secretase inhibitor. Various notch inhibitors are commercially available and include DBZ (cat no. 4489) DAPT (Sigma-Aldrich) LY-685458 (Lilly) and 804929097 (Selleckchem). Integrin inhibitors may target (inhibit) integrin receptors. Integrin inhibitors are commercially available and include RGD peptide (GRGDNP) (ChemCruz Biochemicals). The Hippo pathway is well known in the segregation of the TE and ICM. This pathway ultimately represses a transcription factor (yes-associated protein) YAP, which promotes the TE. Hippo pathway inhibitors are commercially available and well known to the skilled person. For example, the inhibitor may be a Lats inhibitor such as LPA or S1P. LPA and S1P activate YAP/TAZ activity by inhibiting Lats kinase (Yu et al 2012).

Methods and reagents for derivation of EPSCs are described in WO 2016/079146, WO 2020/200071, U.S. Pat. No. 10,745,670, and U.S. Patent Application Publication Nos. 2021/0230556 and 2022/0145264, which are all herein incorporated by reference in their entireties. In some forms, the population of EPSCs is produced by culturing a population of pluripotent stem cells in the EPSCM for one or more (for example, two or more, three or more, four or more, five or more) passages to produce a population of EPSCs. As used herein, “passage” refers to subculturing and is the transfer of cells from a previous culture into fresh growth medium. Cells in culture follow a characteristic growth pattern of lag phase, log phase and stationary phase. The timings of these phases may vary depending on the cell used (e.g., mammalian cells vs non-mammalian cells). Methods to determine the stage of cell growth are well known in the art. Generally, cells are passaged in log phase. In some forms the pluripotent stem cells may be passaged (subcultured) one to ten times, three to ten times, three to five times in the EPSCM, to produce the population of EPSCs. Preferably the population is passaged at least three times to produce the population of EPSCs.

In some forms, the population of EPSCs is produced by culturing cells obtained directly from preimplantation embryos. In an exemplary form, the EPSCs are derived from pre-implantation embryos by contacting the tissue culture plates with eight-cell embryos on feeder cells or feeder-free conditions in cell culture media e.g., M15 ES cell medium, 2i/LIF ES cell medium or EPSCM. Exemplary methods are further described in Yang et al. (2019) Nature Protocols Vol. 14, Issue 2, pages 350.

In some forms, the population of EPSCs is produced by reprogramming somatic cells, for example erythroblasts, to directly generate EPSCs in EPSC medium and without going through an iPSC stage or procedure. In some forms, ES cells and iPS cells can be cultured on SNL76/7 feeders in cell culture media (e.g., M15 ES cell medium or 2i/LIF ES cell medium). In some forms, the conversion process involved plating ES cells or iPS cells at low density on feeders in EPSCM. In some forms, the conversion takes about 15 days, which equates to about five passages. Exemplary methods are further described in Yang et al. (2019) Nature Protocols Vol. 14, Issue 2, pages 350.

In some forms, the population of EPSCs produced are non-human EPSCs, such as for example bovine EPSCs, ovine EPSCs, porcine EPSCs, canine EPSCs, feline EPSCs, equine EPSCs, or primate EPSCs. In preferred forms, the EPSCs are human EPSCs. For example, an exemplary protocol for generating porcine EPSCs is described in WO 2016/0679146A1 by Liu et al., WO 2020/200070 by Liu et al., U.S. application Ser. No. 15/527,269 by Liu et al.; Zhi et al., Cell Research, 32:343-400 (2022); all of which are incorporated herein by reference in their entireties. An exemplary protocol for generating bovine EPSCs is described in Xiang, et al., FEBS J, 288(14):4394-4411 (2021) and WO 2020/200070 by Liu et al., which are incorporated by reference in their entireties.

### IV. Methods of Use

The disclosed compositions provide a readily available source of stem cells that can give rise to a desired cell type or morphology which is typically important for therapeutic treatments, drug discovery, tissue engineering, and research. In preferred forms, the desired cell type is the early STB (eSTBs) or mononucleated STBs. In a preferred form, the eSTBs or mononucleated STBs are human eSTBs. The disclosed eSTBs and compositions thereof are extremely useful in producing coronavirus particles for researching the impact of viral infections, e.g., coronavirus infections on cell viability. The disclosed eSTBs and compositions thereof are also valuable for evaluating/screening drug therapies and vaccines for the treatment and prevention of viral infections, e.g., coronavirus infections.

A. Methods for Culturing Coronavirus Particles

Methods for culturing viral particles in TSCs and their derivatives are disclosed. Typically, the methods include producing a virus by (1) transfecting the disclosed cells with nucleic acids comprising a viral genome, (2) culturing the disclosed cells under conditions that allow replication of the virus, and (3) collecting the virus from the cell culture. In a preferred form, the viral genome is a coronavirus genome, and the virus is coronavirus. Exemplary coronaviruses that may be cultured in the disclosed cells are described elsewhere herein.

In particular, methods for culturing coronavirus particles in eSTBs-containing media are disclosed. The disclosed eSTBs generated from the TSCs provide a source of unlimited physically and clinically relevant cells for isolating and propagating coronaviruses. The disclosed eSTBs permit the production of higher titers of coronaviruses e.g., SARS-CoV-2 and MERS-CoV comparable to that obtained from Vero E6 cells and genetically defective human cancer cells. Importantly, infected eSTBs produced by the disclosed methods have significantly longer survival rates compared to existing cells e.g. Vero E6 cells following viral infection as mechanistically confirmed by morphological and molecular assessments provided in the non-limiting examples.

Typically, the method includes incubating early syncytiotrophoblasts (eSTBs) in coronavirus medium containing coronavirus particles, whereby the coronavirus particles infect and replicate in the eSTBs. In preferred forms, the eSTBs are mononucleated cells and are not multi-nucleated or mature cells. In some forms, the derived eSTBs are isolated by selecting cells expressing eSTB-like morphology. In some forms, the derived eSTBs are isolated by selecting cells expressing an eSTB-like molecular signature. In some forms, the derived eSTBs are isolated by selecting cells expressing both eSTB-like morphology and an eSTB-like molecular signature. The desired morphological and molecular signatures are described above.

1. Propagating the Coronaviruses

In an exemplary form, the coronavirus strain is isolated from a nasopharyngeal aspirate specimen from a coronavirus patient. In some forms, the coronavirus stock is propagated using Vero E6 cells, and the titer of supernatant is assessed by plaque assays.

In a preferred form, eSTBs are the cells that are used for culturing one or more coronaviruses and/or variants thereof. On the day of infection, cells are washed with PBS and infected at the indicated multiplicity of infection (MOI) by diluting viruses in basal medium. Preferably, cells are incubated at 37° C. for 2 hours. Subsequently, the inoculum is removed, replaced with complete culture medium, and further incubated until harvest. In an exemplary form, the eSTBs are co-incubation with one or more coronaviruses or variants thereof for between about 1 to about 3 days. In some forms, one or more coronaviruses and/or variants thereof may be propagated in other trophoblast cells and trophoblast precursors as controls for comparative analyses with coronavirus-infected eSTBs. In some forms, non-limiting examples of trophoblast cells and trophoblast precursors include but are not limited to mature or multinucleated STBs, extravillous cytotrophoblasts (EVTs) such as interstitial EVTs and endovascular EVTs, and TSCs.

In preferred forms, cytopathic effects (CPE) are monitored daily via light microscopy, and cell supernatant and lysates at specific time points are collected for RT-qPCR to assess the viral RNA load. Cytopathic effects that may be monitored include total destruction of the cell monolayer, subtotal destruction of the cell monolayer, focal degeneration, swelling and clumping of the cells, vacuolization or foamy degeneration, cell fusion and polykaryon formation, and formation of inclusion bodies. In some forms, plaque assays are used to calibrate the viral RNA load to viral load determination.

2. Detection of the Coronavirus and its Effects in eSTBs

Typically, the supernatants of the cultured cells that are challenged by SARS-CoV-2 are collected at various time points for viral detection. Methods of detecting viral load and viral replication are well known in the art.

Exemplary methods of detecting the effects of the coronavirus on the eSTBs is described in the non-limiting Examples. In some forms, the coronavirus-infected and uninfected eSTBs are evaluated using PCR techniques, immunoassays, sequencing, biochemical assays, functional assays, cell viability assays, microscopy, or combinations thereof. In a preferred form, AVL buffer is added to a volume of cell culture supernatant. AVL buffer is a viral lysis buffer used for purifying viral nucleic acids. Then, total RNA is extracted using a viral RNA kit. Kits for extracting viral RNA are well known in the art and include for example, the RNA Mini Kit (Qiagen, Catalog #52906); Invitrogen™ PureLink™ Viral RNA/DNA Mini Kit (Catalog #12280050), AccuPrep® Viral RNA Extraction Kit (Catalog #K-3033G), and E.Z.N.A.® Viral RNA Kit (Catalog #R6874).

In some forms, a portion of the isolated eSTBs is assessed for coronavirus susceptible markers. In some forms, the coronavirus susceptible markers are increased ACE2 and/or increased TMPRSS2. In a preferred form, the coronavirus susceptible marker ACE2 is identified via RT-qPCR using the primers SEQ ID NO:2 and SEQ ID NO:52, SEQ ID NO:3 and SEQ ID NO:53, or SEQ ID NO:96 and SEQ ID NO:97, and/or immunofluorescence staining. In another preferred form, the coronavirus susceptible marker TMPRSS2 is identified via RT-qPCR using the primers SEQ ID NO:4 and SEQ ID NO:54.

In some forms, methods for detection of the one or more coronaviruses and their effects on the eSTBs include quantifying the coronavirus load in the coronavirus medium. In some forms, the assessment of the portion of the eSTBs for coronavirus susceptible markers is done via virus replication kinetic assays. In some forms, the virus replication kinetic assays are RT-qPCR, plaque assays, Trans-well invasion assays, and/or RNA sequencing. In exemplary form, when the coronavirus particles as SARS-CoV-2 particles, the coronavirus load is detected via RT-qPCR using the primers SEQ ID NO:1 and SEQ ID NO:51.

In an exemplary virus replication kinetics assay, the extracted RNA is quantified with the one-step QuantiNova Probe RT-PCR kit (Qiagen, Catalog #208354). In some forms, each reaction mixture typically contains an effective amount of 2×QuantiNova Probe RT-PCR Master Mix, QuantiNova Probe RT-Mix, a forward and reverse primer, a probe, the extracted RNA as template, and RNase-free water. An exemplary amplification step includes incubating the reactions at 45° C. for 10 minutes for reverse transcription, 95° C. for 5 minutes for denaturation, 45 cycles of 95° C. for 5 seconds and 55° C. for 30 seconds, followed by a cooling step at 40° C. for 30 seconds. The primers and probe sequences are typically designed to detect the RNA-dependent RNA polymerase/helicase (RdRP/Hel) gene region of the SARS-CoV-2 virus. Exemplary primer sequences are listed in Tables 1A and 1B. Methods of designing primer and probe sequences for viral detection are known in the art.

3. Providing Cells for Propagating High Titers of Viruses for Vaccine Production

It has been established that the eSTBs are highly susceptible to infections by coronaviruses such as SARS-CoV-2 and MERV-CoV. Thus, the disclosed compositions and methods are useful for efficient propagation and harvesting of high titers of coronaviruses for vaccine production. In particular, the eSTBs and compositions and methods thereof, overcome defects of existing technology, using the Vero cell derived from green monkey to produce the coronaviruses and prepare the inactivated vaccines. In preferred forms, the disclosed cells such as the early STBs, will not die following viral infection and virus replication, thereby rendering the disclosed cells e.g. early STBs, highly efficient experimental models compared to Vero cells.

In some forms, the disclosed cells can be re-used for virus replication and virus harvest.

Typically, the coronaviruses are produced by (1) transfecting the disclosed cells with nucleic acids isolated from a coronavirus genome, (2) culturing the disclosed cells under conditions that allow replication of the coronavirus, and (3) collecting the coronavirus from the cell culture.

The term “transfection” or “transfecting” refers to the introduction of a foreign nucleic acid into a cell so that the host cell will express the introduced gene or sequence to produce a desired polypeptide, coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including the same genus or species as the host cell, or from a different genus or species.

In an exemplary form, high viral titers are obtained by: (1) washing the disclosed cells with PBS; (2) infecting the disclosed cells at a desired multiplicity of infection (MOI) by diluting the coronavirus in basal medium; (3) incubating the disclosed cells with the coronavirus at a desired temperature e.g., about 37° C. for a period of time e.g., about 2 hours; (4) removing the inoculum and replacing with complete culture medium, and incubating until harvest of the viruses; and (5) optionally, quantifying viral load via a viral detection and/or quantification assay e.g., RT-qPCR and/or plaque assays.

In some forms, the harvested coronavirus can be whole coronavirus for the production of inactivated vaccines, live attenuated vaccines, and/or viral vector vaccines. Inactivated vaccines are created by inactivating a virus, typically using heat or chemicals such as formaldehyde or formalin, thereby destroying the virus' ability to replicate, but keeps the virus “intact” so that the immune system can still recognize it. Inactivated viruses cannot replicate, they cannot revert to a more virulent form capable of causing a viral infection. A live-attenuated vaccine uses a living but weakened version of the coronavirus or a virus similar to the coronavirus. A viral vector vaccine uses a “safe” virus to deliver specific sub-parts—called proteins—of the coronavirus of interest so that it can trigger an immune response without causing disease. To do this, the instructions for making particular parts of the coronavirus of interest are inserted into a “safe” virus. The “safe” virus then serves as a platform or vector to deliver the protein into the body, whereby the protein triggers the immune response.

In some forms, the harvested coronavirus can be a subunit vaccine. In some forms, the subunit vaccine contains only specific parts (the subunits) of the coronavirus that the immune system needs to recognize. A subunit vaccine does not contain the whole coronavirus, nor does it use a safe virus as a vector. In some forms, the subunits may be proteins and/or sugars.

In some forms, the harvested coronavirus can be a nucleic acid vaccine. In some forms, the nucleic acid vaccine contains only a section of genetic material e.g., DNA and RNA, that provides the instructions for specific proteins, not the whole coronavirus.

B. Drug Screening and Evaluation

The establishment of the TSCs, and their derivatives, eSTBs and EVTs, open the avenue to generate the cell types of interest for high throughput screening. For example, in some forms, the disclosed cells can be used to screen drug safety, toxicity, efficacy and to evaluate test compounds for drug therapies.

In some forms, the disclosed cells can be used as a platform for determining the effect of candidate compounds on the inhibition of the coronavirus prior to evaluating of the efficacy of the candidate compound or the approval of the candidate compound for further drug testing to determine drug safety.

In some forms, the disclosed cells can be used to determine the drug efficacy for existing drugs e.g., FDA approved drugs, or the compound newly discovered, whether it is effective in clinical usage might be evaluated using eSTB according to clinical symptom or other testing for therapies.

In an exemplary form, the disclosed cells can serve as a high-throughput platform in drug discovery to prevent pregnancy failure and birth defects. For example, in some forms, TSCs and their derivatives, e.g., eSTBs can be generated from the established differentiation protocol and adopted as a screening platform to identify compounds with antiviral properties that are safe for administration to pregnant subjects.

In a second exemplary form, compounds are tested for toxicity and/or the ability to further improve one or more wildtype functions. For example, in some forms, TSCs and their derivatives (e.g., eSTBs) can be generated from the established differentiation protocol and adopted as a screening platform to identify one or more compounds that are compatible with the development of inactivated vaccines.

In some forms, cells are cultured under conditions suitable to induce differentiation of the desired cell population as disclosed herein. In some forms, one or more test compounds can be applied to cultured differentiated cells and evaluated for the ability to treat one or more symptoms of the diseased, dysfunctional, or defective cells.

The symptom or symptoms can be specific to the disease state being studied or can be of a general nature. Physiological, phenotypic, morphological, or molecular symptoms and other markers of the cells can be monitored over time.

In an exemplary form, the disclosed cells, and compositions thereof, are useful for investigating the activity or applicability of one or more test compounds to treat or alleviate one or more symptoms of a viral infection and/or a disease or disorder associated with a viral infection. In these forms, the test compounds can be added at different stages of viral infection e.g., pre-infection (before introducing the virus) or post-infection (before introducing the virus). In some forms, test compounds might also be pre-incubated with the virus (neutralization). In these forms, various tests can help reveal how an antiviral works (e.g., does it neutralize the virus or does it stop viral entry).

1. Evaluation/Screening of Antiviral Agents

There is a lack of effective treatments available for the treatment of coronaviruses. The disclosed compositions and methods are useful for investigating the activity or efficacy of one or more test compounds to treat or alleviate or prevent one or more symptoms of any one or more of the coronaviruses described above in Section II (B).

In some forms, the disclosed coronavirus infected eSTBs may be used to screen an agent for an effect on coronavirus infected cells, the method including contacting the coronavirus infected eSTBs with the agent and determining the effect of the agent on survival, proliferation, differentiation, or morphologic, genetic, or functional parameters of the eSTBs. Therefore, in some forms, the methods include one or more steps for assessing the replication of a coronavirus in the presence of one or more active agents.

In some forms, the agent could be an existing FDA-approved drug, with known indications. In some forms, the agent is an unknown compound that has not been reported for its antiviral effect. In some forms, the agent could be one or more chemically synthesized compounds. In some forms, the agent could be one or more components extracted from natural plants or herbs.

In some forms, the agent is an antiviral agent, wherein the effect of the agent is indicative of the agent being safe for treatment of viral infections. In some forms, the effect of the agent is indicative of the agent being safe for treatment of a pregnant subject infected with coronavirus, a fetus infected with coronavirus, or both a pregnant subject infected with coronavirus and a fetus infected with coronavirus. In some forms, the agent may be nucleic acids or analogs thereof, polypeptides or analogs thereof, antibodies, chemicals, small molecules, and/or any combination thereof. In some forms, the treated and untreated coronavirus-infected eSTBs are evaluated using PCR techniques, immunoassays, sequencing, biochemical assays, functional assays, cell viability assays, microscopy, or combinations thereof. Methods for screening agents are known in the art and include but are not limited to scintillation proximity assays, Direct fluorescence measurement, Fluorescence polarization, Fluorescence resonance energy transfer (FRET), Time-resolved fluorescence (TRF, HTRF, and TiRF), AlphaScreen, High-content screening (HCS), Protein fragment complementation assays (PCA), microfluidics, flow cytometry, and label-free technologies (reviewed in Janzen (2014) Chemistry and Biology Vol 21(9), pages 1162-1170).

2. Therapeutic Agents

Remdesivir (GS-5734), an inhibitor of the viral RNA-dependent, RNA polymerase with in vitro inhibitory activity against SARS-CoV-1 and the Middle East respiratory syndrome (MERS-CoV), was identified early as a promising therapeutic candidate for COVID-19 because of its ability to inhibit SARS-CoV-2 in vitro. On Oct. 22, 2020, the U.S. Food and Drug Administration (FDA) approved the antiviral drug VEKLURY® (remdesivir) for use in adults and pediatric patients (12 years of age and older and weighing at least 40 kg) for the treatment of COVID-19 requiring hospitalization.

The FDA has issued an Emergency Use Authorization (EUA) on Dec. 22, 2021 for Pfizer's PAXLOVID™ (nirmatrelvir tablets and ritonavir tablets, co-packaged for oral use) for the treatment of mild-to-moderate coronavirus disease 2019 (COVID-19) in adults and pediatric patients (12 years of age and older weighing at least 40 kg) with positive results of direct severe acute respiratory syndrome coronavirus 2 (SARS-CoV-2) viral testing, and who are at high risk for progression to severe COVID-19, including hospitalization or death. PAXLOVID™ consists of nirmatrelvir, which inhibits a SARS-CoV-2 protein to stop the virus from replicating, and ritonavir, which slows down nirmatrelvir's breakdown to help it remain in the body for a longer period at higher concentrations. PAXLOVID™ is administered as three tablets (two tablets of nirmatrelvir and one tablet of ritonavir) taken together orally twice daily for five days, for a total of 30 tablets. PAXLOVID™ is not authorized for use for longer than five consecutive days.

Monoclonal antibody therapies remain available under EUA, including REGEN-COV® (casirivimab and imdevimab, administered together), and bamlanivimab and etesevimab, administered together.

FDA has authorized the emergency use of baricitinib to treat COVID-19 in hospitalized adults and pediatric patients 2 years or older requiring supplemental oxygen, non-invasive or invasive mechanical ventilation, or extracorporeal membrane oxygenation (ECMO). According to a statement issued by the WHO on January 14, Baricitinib is recommended for treating patients suffering with severe or critical Covid-19. On the other hand, Sotrovimab, a monoclonal antibody drug, is recommended for treating patients who have mild or moderate Covid-19.

Accordingly, in some forms, the methods involve the step of contacting the coronavirus infected eSTBs with one or more of antiviral drugs such as remdesivir and PAXLOVID™, and monoclonal antibodies such as casirivimab, imdevimab, bamlanivimab, etesevimab, baricitinib, and sotrovimab, and determining the effect of the agent on survival, proliferation, differentiation, or morphologic, genetic, or functional parameters of the eSTBs based on the disclosed methods. In some forms, the treated and untreated coronavirus-infected heSTBs are evaluated using PCR techniques, immunoassays, sequencing, biochemical assays, functional assays, cell viability assays, microscopy, or combinations thereof.

C. Providing Cells for Studying the Effects of Infectious Agents on Early Embryonic Development

The study of mammalian embryonic development from fertilized egg to mature embryo is extremely important but the early differentiation of human and non-human tissues remains an enigma. Moreover, the relatively high incidence of pregnancy-related complications associated with viral infections, such as miscarriage, restricted fetal growth, or still-birth emphasizes the need for an appropriate model for studying the effects of viral infections on early embryonic development. In some forms, the disclosed cells are particularly suited for use to investigate the impact of viruses in previously inaccessible basic processes that occur during early embryogenesis, such as gastrulation and organogenesis. In some forms, the disclosed cells are used to investigate the impact of viruses on embryonic development in human cells. In some forms, the disclosed cells are used to investigate the impact of viruses on embryonic development in non-human cells e.g., non-human primate cells and other non-human mammalian cells such as porcine cells, mouse cells, and rat cells.

It is shown that coronaviruses primarily infect the early syncytiotrophoblast cells and the other maternal-fetal interface cells because ACE2 is significantly expressed in these maternal-fetal interface cells. Thus, in some forms, the disclosed cells can be used to investigate the immunopathology of coronavirus infections in pregnant subjects. In some forms, the disclosed cells can be used to investigate the impact of coronavirus infections on fetal development, e.g., abnormalities in the developing liver, oddities in the development of the nervous system, and various complications in the heart and kidneys. In some forms, the disclosed cells can be used to investigate the influence of demographic differences on the immunopathology of coronavirus infections in pregnant subjects and fetal development.

The use of the disclosed cells for such studies would also obviate the need for mammalian embryos and usage of ethically challenging cell sources. Thus, in some forms, the disclosed compositions are useful in cell models in the preclinical study of infertility-related diseases such as infertility, failure of implantations, and preeclampsia.

D. Gene and Genome Editing

Viral mutation presents a challenge in the efficient development of effective vaccines, anti-viral therapies, and other therapeutic treatments for coronavirus infections. For example, the longer the SARS-CoV-2 virus replicates in hosts, the more likely it will develop new ways to spread in the face of existing natural immunity, vaccines, and treatments. Thus, public health efforts to prevent the spread of the virus, including increased vaccinations, is crucial both to prevent illness and reduce coronavirus replication and transmission.

The disclosed cells, compositions, and methods are useful for genome editing of various coronaviruses. In some forms, the cells, compositions, and methods can be used to generate coronaviruses and/or coronavirus particles with mutations in target genes for disease modeling studies, vaccine production, and/or drug discovery.

The disclosed cells, compositions, and methods are also useful for testing the effect of host mutations on the susceptibility to coronavirus infections. In some forms, the disclosed cells are used to test the impact of host mutations on coronavirus binding to host cell receptors and/or entry into host cells. In some forms, the disclosed cells are used to test the impact of host mutations on coronavirus replication within host cells e.g., viral RNA synthesis, virion assembly, and/or viral release. These may be useful for the identification of druggable targets.

The disclosed cells, compositions, and methods are also useful for evaluating genome editing therapies and technologies for the treatment of coronavirus infections and/or conditions or diseases associated with coronavirus infections. For example, CRISPR/Cas13 system targets RNA and may have potential in treating RNA viral infectious diseases, e.g., SARS-CoV-2. In an exemplary form, the disclosed cells and compositions thereof can be used to grow SARS-CoV-2 viruses for targeting of the conserved regions of the SARS-CoV-2 genome using a gRNA, Cas13, which can cut and clear the viral RNA genome.

The present invention can be further understood by the following non-limiting paragraphs:

1. A method comprising incubating early syncytiotrophoblasts (eSTBs) in coronavirus medium comprising coronavirus particles, whereby the coronavirus particles infect and replicate in the eSTBs.

2. The method of paragraph 1, wherein the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles, non-human coronavirus particles and/or variant particles thereof.

3. The method of paragraph 1, wherein the coronavirus particles are non-human coronavirus particles selected from the group comprising canine enteric coronavirus (CECoV) particles, feline coronavirus (FCoV) particles, porcine respiratory coronavirus (PRCV) particles, porcine epidemic diarrhea virus (PEDV) particles, transmissible gastroenteritis virus (TGEV) particles, canine respiratory coronavirus (CRCoV) particles, murine coronavirus (M-CoV) particles, porcine hemagglutinating encephalomyelitis virus (PHEV) particles, porcine enteric coronavirus (PEC) particles, swine acute diarrhea syndrome coronavirus (SADS-CoV), porcine delta coronavirus (PDCoV), hedgehog coronavirus 1 particles, bovine coronavirus (B-CoV) particles, equine coronavirus (E-CoV) particles, tylonycteris bat coronavirus HKU4 (Bat-CoV HKU4) particles, pipistrellus bat coronavirus HKU5 (Bat-CoV HKU5) particles, rousettus bat coronavirus HKU9 (Bat-CoV HKU9) particles, Avian Infectious Bronchitis (AIBV) particles, Beluga Whale CoV SW1 coronavirus particles and variant particles thereof.

4. The method of any one of paragraphs 1-3, wherein the coronavirus particles are SARS-CoV-2 particles.

5. The method of any one of paragraphs 2-4, wherein the SARS-CoV-2 particles are SARS-CoV-2 alpha variant particles, SARS-CoV-2 beta variant particles, SARS-CoV-2 gamma variant particles, SARS-CoV-2 delta variant particles, SARS-CoV-2 epsilon variant particles, SARS-CoV-2 eta variant particles, SARS-CoV-2 iota variant particles, SARS-CoV-2 kappa variant particles, SARS-CoV-2 mu variant particles, SARS-CoV-2 omicron variant particles, SARS-CoV-2 zeta variant particles, SARS-CoV-2 1.617.3 variant particles and/or SARS-CoV-2 lambda variant particles.

6. The method of any one of paragraphs 1-5, wherein the eSTBs are mononucleated cells.

7. The method of any one of paragraphs 1-5, wherein the eSTBs are not multi-nucleated or mature cells.

8. The method of any one of paragraphs 1-7, wherein the derived eSTBs are isolated by selecting cells expressing eSTB-like morphology, cells expressing an eSTB-like molecular signature, or cells expressing both eSTB-like morphology and an eSTB-like molecular signature.

9. The method of paragraph 8, wherein the eSTB-like molecular signature comprises the increased early STB markers CD46 and/or SSEA4; one or more increased STB markers, wherein the increased STB markers are GCM1, β chorionic gonadotrophin 3 gene (CGB3), CGB5, CD46, ENG, and/or CSH2, decreased mature STB markers such as trophoblast progenitor transcription factor TP63, and/or properly folded or secreted (3-hCG hormone.

10. The method of paragraph 8 or 9 further comprising assessing a portion of the isolated eSTBs for coronavirus susceptible markers.

11. The method of paragraph 10, wherein the coronavirus susceptible markers are increased ACE2 and/or increased TMPRSS2.

12. The method of paragraph 10 or 11, wherein the assessment of the portion of the eSTBs for coronavirus susceptible markers is done via virus replication kinetic assays.

13. The method of paragraph 12, wherein the virus replication kinetic assays are RT-qPCR, plaque assays, Trans-well invasion assays, and/or RNA sequencing.

14. The method of any one of paragraphs 11-13, wherein the coronavirus susceptible marker ACE2 is identified via RT-PCR or RT-qPCR using the primers SEQ ID NO:2 and SEQ ID NO:52, SEQ ID NO:3 and SEQ ID NO:53, or SEQ ID NO:96 and SEQ ID NO:97.

15. The method of any one of paragraphs 11-13, wherein the coronavirus susceptible marker TMPRSS2 is identified via RT-qPCR using the primers SEQ ID NO:4 and SEQ ID NO:54.

16. The method of any one of paragraphs 1-15 further comprising quantifying the coronavirus load in the coronavirus medium.

17. The method of paragraph 16, wherein quantifying the coronavirus load is done via a plaque assay, RT-qPCR, and/or RNA sequencing.

18. The method of paragraph 16 or 17, wherein the coronavirus particles as SARS-CoV-2 particles, wherein the coronavirus load is detected via RT-qPCR using the primers SEQ ID NO:1 and SEQ ID NO:51.

19. The method of any one of paragraphs 1-18 further comprising assessing the derived eSTBs for cytopathic effects.

20. The method of any one of paragraphs 1-19, wherein the eSTBs are incubated with the coronavirus medium comprising the coronavirus particles at about 37° C. for about 1 day to 6 days, preferably for about 2 days.

21. The method of any one of paragraphs 1-20, wherein the coronavirus particles were diluted in the coronavirus medium.

22. The method of any one of paragraphs 1-21, wherein the coronavirus medium comprises basal medium, wherein basal medium is DMEM/F-12 or DMEM.

23. The method of any one of paragraphs 1-22, wherein the coronavirus particles were isolated from a sample comprising the coronavirus particles.

24. The method of paragraph 23, wherein the sample comprising the coronavirus particles is coronavirus-infected VeroE6 cells.

25. The method of paragraph 23, wherein the sample comprising the coronavirus particles is a directly obtained patient sample.

26. The method of any one of paragraphs 1-25, wherein the eSTBs are incubated in the coronavirus medium for about 1-6 days, preferably for about 2 days.

27. The method of any one of paragraphs 1-26, wherein the eSTBs are derived from trophoblast stem cells (TSCs).

28. The method of paragraph 27, wherein the TSCs are human TSCs, bovine TSCs, ovine TSCs, porcine TSCs, canine TSCs, feline TSCs, equine TSCs, or primate TSCs.

29. The method of paragraph 27 or 28, wherein the eSTBs are derived by culturing the TSCs in STB medium for about 1 to about 6 days.

30. The method of paragraph 29, wherein the STB medium comprises basal medium supplemented with one or more of a reducing agent, BSA, an antibiotic, a ROCK inhibitor, a cAMP inhibitor, KSR medium, and/or one or more differentiation agents, wherein basal medium is DMEM/F-12 or DMEM.

31. The method of paragraph 30, wherein the antibiotic is Penicillin-Streptomycin-Glutamine at about 0.5 weight percent.

32. The method of paragraph 30, wherein the reducing agent is β-mercaptoethanol in a concentration of about 50 μM.

33. The method of paragraph 30, wherein the ROCK inhibitor is Y-27632 in a concentration of about 2.5 μM.

34. The method of paragraph 30, wherein the cAMP inhibitor is forskolin in a concentration of about 2 μM.

35. The method of paragraph 30, wherein the differentiation agent is ITS-X at about 1%.

36. The method of any one of paragraphs 27-35, wherein the number of TSCs used to derive the eSTBs is about 0.5×10⁵/cm² to about 2.0×10⁵/cm², preferably about 1.0×10⁵/cm² TSCs.

37. The method of any one of paragraphs 27-36, wherein the TSCs are derived from expanded potential stem cells (EPSCs), primed stem cells, naïve stem cells, embryonic stem cells, induced pluripotent stem cells, other pluripotent stem cells, peri-implantation embryos, placental tissues and/or genetically altered derivatives thereof.

38. The method of paragraph 37, wherein when EPSCs are used to derive the TSCs, the TSCs are derived by:
(i) culturing dissociated EPSCs for about 24 hours in a first culture medium comprising one or more of knock-out serum replacement (KSR) medium, growth factors, and a ROCK inhibitor;
(ii) culturing the EPSCs from step (i) in a second culture medium comprising a TGF-β inhibitor and KSR medium; and
(iii) culturing the EPSCs from step (ii) in a third culture medium comprising human trophoblast-induced stem cell medium, thereby producing TSC colonies.

39. The method of paragraph 38, wherein the derived TSCs are identified by screening the TSC colonies for expression of TSC-like morphology.

40. The method of any of paragraphs 35-39, wherein the derived TSCs are identified by screening the TSC colonies for expression of an TSC-typical molecular signature.

41. The method of paragraph 40, wherein the hTSC-typical molecular signature is increased TSC factors, increased trophoblast-specific miRNAs, decreased HLA class I molecules, and/or decreased AME genes.

42. The method of paragraph 41, wherein the increased TSC factors are TFAP2C, TP63, CK18, GATA3, ELF5, TEAD4, and/or KRT7.

43. The method of paragraph 41, wherein the increased trophoblast-specific miRNAs are has-miR-517c-3p, 517-5p, 525-3p, and/or 526b-3p.

44. The method of paragraph 41, wherein the decreased HLA class I molecules are HLA-A and/or HLA-B.

45. The method of paragraph 41, wherein the decreased AME genes are CDX2, MUC16, GABRP, ITGB6, and/or VTCN1.

46. The method of any one of paragraphs 37-45 further comprising re-plating the TSCs.

47. The method of any one of paragraphs 37-46 further comprising passaging the TSCs one or more times.

48. The method of any one of paragraphs 38-47, wherein the EPSCs are dissociated into single EPSCs in a dissociating reagent.

49. The method of paragraph 48, wherein the dissociating reagent comprises a trypsin replacement agent.

50. The method of any one of paragraphs 38-49, wherein the number of EPSCs used in the first culture medium is about 0.5×10⁵ cells to about 2.0×10⁵ cells per well of a 6-well cell culture dish, preferably about 1×10⁵ cells per well.

51. The method of any one of paragraphs 38-50, wherein the ROCK inhibitor is Y27632 or thiazovivin.

52. The method of any one of paragraphs 38-51, wherein the first culture medium comprises the ROCK inhibitor in a concentration of at least about 2 μM to about 10 PM.

53. The method of any one of paragraphs 38-52, wherein the TGF-β inhibitor is SB431542 or A83-01 in a concentration of about 2 μM to about 10 μM.

54. The method of any one of paragraphs 38-53, wherein the number of hEPSCs used in the third culture medium is about 2000 cells per well to about 10,000 cells per well.

55. The method of any one of paragraphs 38-54, wherein the trophoblast stem cell medium comprises basal medium supplemented with one or more of a reducing agent, fetal bovine serum (FBS), an antibiotic, Bovine Serum Albumin (BSA), Epidermal Growth Factor (EGF), Glycogen synthase kinase 3 (GSK-3) inhibitor, an ALK-5 inhibitor, a ROCK inhibitor, a TGF-β inhibitor, and/or an HDAC inhibitor, wherein basal medium is DMEM/F-12 or DMEM.

56. The method of paragraph 55, wherein the reducing agent is β-mercaptoethanol in a concentration of about 10.0 μM to about 100.0 μM, preferably about 50.0 μM.

57. The method of paragraph 55 or 56, wherein the trophoblast stem cell medium comprises basal medium supplemented with about 10% FBS, wherein basal medium is DMEM/F-12 or DMEM.

58. The method of any one of paragraphs 55-57, wherein the antibiotic is penicillin-Streptomycin-Glutamine at a weight percent of about 1%.

59. The method of any one of paragraphs 55-57, wherein the Epidermal Growth Factor is present in the trophoblast-induced stem cell medium at a concentration of about 50.0 ng/mL.

60. The method of any one of paragraphs 55-59, wherein the GSK-3 inhibitor is CHIR99021 in a concentration of about 2.0 μM.

61. The method of any one of paragraphs 55-60, wherein the ALK-5 inhibitor is A83-01 in a concentration of about 0.5 μM.

62. The method of any one of paragraphs 55-61, wherein the TGF-β inhibitor is SB431542 in a concentration of about 1.0 μM.

63. The method of any one of paragraphs 55-62, wherein the HDAC inhibitor is valproic acid at a concentration of about 10.0 μM.

64. The method of any one of paragraphs 37-63, wherein the EPSCs are derived from pluripotent stem cells.

65. The method of paragraph 64, wherein the EPSCs are derived by culturing the pluripotent stem cells in a fourth culture medium for a time period sufficient to derive EPSCs.

66. The method of paragraph 65, wherein the fourth culture medium comprises one or more of a RAS-ERK inhibitor, a SRC kinase inhibitor, a GSK3 inhibitor, and a WNT inhibitor.

67. The method of any one of paragraphs 64-66 further comprising passaging EPSCs in an EPSC maintenance medium for about 3 to 5 passages.

68. The method of any one of paragraphs 64-67 further comprising maintaining the derived EPSCs in an EPSC maintenance medium.

69. The method of paragraph 67 or 68, wherein the EPSC maintenance medium comprises one or more of a RAS-ERK inhibitor, a SRC kinase inhibitor, a GSK3 inhibitor, a WNT inhibitor, and/or activin.

70. The method of paragraph 69, wherein the SRC inhibitor is A-419259, XAV939, a Tankyrase inhibitor, or a combination thereof.

71. The method of any one of paragraphs 64-70, wherein the pluripotent stem cells are embryonic stem cells (ESCs) or induced pluripotent stem cells (iPSCs) or other types of pluripotent stem cells that can generate TSCs.

72. One or more coronavirus infected eSTBs produced by the method of any one of paragraphs 1-71.

73. A method of screening an agent for an effect on coronavirus infected cells, the method comprising contacting the coronavirus infected eSTBs of paragraph 1 with the agent and determining the effect of the agent on survival, proliferation, differentiation, or morphologic, genetic, or functional parameters of the eSTBs.

74. The method of paragraph 73, wherein the effect of the agent is indicative of the agent being safe for treatment of a pregnant subject infected with coronavirus, a fetus infected with coronavirus, or both.

75. The method of paragraph 73, wherein the agent is an antiviral agent, wherein the effect of the agent is indicative of the agent being safe for treatment of viral infections.

76. The method of any one of paragraphs 73-75, wherein the agent is nucleic acids or analogs thereof, polypeptides or analogs thereof, antibodies, chemicals, small molecules, and/or any combination thereof.

77. The method of any one of paragraphs 73-76, wherein the treated and untreated coronavirus-infected eSTBs are evaluated using PCR techniques, immunoassays, sequencing, biochemical assays, functional assays, cell viability assays, microscopy, or combinations thereof.

78. The method of any one of paragraphs 73-77, wherein the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles, non-human coronavirus particles and/or variant particles thereof.

79. The method of any one of paragraphs 73-78, wherein the coronavirus particles are SARS-CoV-2 particles.

80. The method of any one of paragraphs 73-79, wherein the SARS-CoV-2 particles are SARS-CoV-2 alpha variant particles, SARS-CoV-2 beta variant particles, SARS-CoV-2 gamma variant particles, SARS-CoV-2 delta variant particles, SARS-CoV-2 epsilon variant particles, SARS-CoV-2 eta variant particles, SARS-CoV-2 iota variant particles, SARS-CoV-2 kappa variant particles, SARS-CoV-2 mu variant particles, SARS-CoV-2 omicron variant particles, SARS-CoV-2 zeta variant particles, SARS-CoV-2 1.617.3 variant particles and/or SARS-CoV-2 lambda variant particles.

81. The method of any one of paragraphs 73-79, wherein the coronavirus particles are non-human coronavirus particles selected from the group comprising canine enteric coronavirus (CECoV) particles, feline coronavirus (FCoV) particles, porcine respiratory coronavirus (PRCV) particles, porcine epidemic diarrhea virus (PEDV) particles, transmissible gastroenteritis virus (TGEV) particles, canine respiratory coronavirus (CRCoV) particles, murine coronavirus (M-CoV) particles, porcine hemagglutinating encephalomyelitis virus (PHEV) particles, porcine enteric coronavirus (PEC) particles, swine acute diarrhea syndrome coronavirus (SADS-CoV), porcine delta coronavirus (PDCoV), hedgehog coronavirus 1 particles, bovine coronavirus (B-CoV) particles, equine coronavirus (E-CoV) particles, tylonycteris bat coronavirus HKU4 (Bat-CoV HKU4) particles, pipistrellus bat coronavirus HKU5 (Bat-CoV HKU5) particles, rousettus bat coronavirus HKU9 (Bat-CoV HKU9) particles, Avian Infectious Bronchitis (AIBV) particles, Beluga Whale CoV SW1 coronavirus particles and variant particles thereof.

82. A method for screening an agent for an effect on early syncytiotrophoblasts (eSTBs), the method comprising contacting the eSTBs with the agent and determining the effect of the agent on survival, proliferation, differentiation, or morphologic, genetic, or functional parameters of the eSTBs.

83. The method of paragraph 82, wherein the effect of the agent is indicative of the agent being safe for treatment of a subject.

84. The method of paragraph 82, wherein the effect of the agent is indicative of the agent being safe for treatment of a pregnant subject, a fetus infected with coronavirus, or both.

85. The method of paragraph 82, wherein the agent is an antiviral agent, wherein the effect of the agent is indicative of the agent being safe for treatment of viral infections.

86. The method of any one of paragraphs 82-85, wherein the agent is nucleic acids or analogs thereof, polypeptides or analogs thereof, antibodies, chemicals, small molecules, and/or any combination thereof.

87. The method of any one of paragraphs 82-86, wherein the treated and untreated eSTBs are evaluated using PCR techniques, immunoassays, sequencing, biochemical assays, functional assays, cell viability assays, microscopy, or combinations thereof.

88. A kit for culturing the coronavirus particles in early syncytiotrophoblasts (eSTBs), the kit comprising a combination of two or more of:
(i) the fourth culture medium for deriving the EPSCs described in A(ii) comprising one or more of a RAS-ERK inhibitor, a SRC kinase inhibitor, a GSK3 inhibitor, and/or a WNT inhibitor;
(ii) the EPSC maintenance medium comprising one or more of a RAS-ERK inhibitor, a SRC kinase inhibitor, a GSK3 inhibitor and/or a WNT inhibitor; (iii) the dissociating reagent for producing single EPSCs comprising a trypsin replacement agent;
(iv) the trophoblast stem cell medium comprising basal medium supplemented with one or more of a reducing agent, fetal bovine serum (FBS), an antibiotic, Bovine Serum Albumin (BSA), Epidermal Growth Factor (EGF), Glycogen synthase kinase 3 (GSK-3) inhibitor, an ALK-5 inhibitor, a ROCK inhibitor, a TGF-β inhibitor, and/or an HDAC inhibitor, wherein basal medium is DMEM/F-12 or DMEM;
(v) STB medium comprising basal medium supplemented with one or more of a reducing agent, BSA, an antibiotic, a ROCK inhibitor, a cAMP inhibitor, KSR medium, and/or one or more differentiation agents; and
(vi) coronavirus medium comprising basal medium, wherein basal medium is DMEM/F-12 or DMEM.

The present invention can be further understood by the following non-limiting examples.

### EXAMPLES

### Example 1: Establishment of EPSC-TSCs and Generation of STBs and EVTs for Infection

### Materials and Methods

Data and Code Availability

RNA-seq data generated in this study have been deposited at NCBI Gene Expression Omnibus (GEO): GSE190432.

Experimental Model and Subject Details

### Human Expanded Potential Stem Cells (hEPSCs) and Cell Lines

Human embryonic stem cells (hESCs) Man-1/M1 were converted to hEPSCs, as described in previous studies (Gao, X. et al. (2019) Nature Cell Biology 21(6), pages 687-699). Human EPSC cultures were routinely maintained on STO feeders. Irradiation inactivated STO cells were prepared 3-4 days before seeding hEPSCs on 0.1% gelatinized plates at the density of ˜3.125×10⁴ cells/cm². STO cells were maintained in regular M10 medium: knockout DMEM, 10% FBS, 1×Glutamine Penicillin-Streptomycin and 1×Minimum Essential Medium (MEM) Vitamin Solution. Human colon Caco-2 cells and monkey Vero E6 cells were maintained in DMEM culture medium supplemented with 10% heat-inactivated FBS, 50 Uml⁻¹ penicillin and 50 μgml⁻¹ streptomycin. All cells were maintained in a 5% CO₂ incubator at 37° C. and routinely tested for mycoplasma.

Virus

The SARS-CoV-2 HKU-001a strain (GenBank accession number: MT230904) was isolated from the nasopharyngeal aspirate specimen of a patient who was laboratory-confirmed to have COVID-19 in Hong Kong (Chan, J. F. et al. Clinical Infectious Diseases 71(9), pages 2428-2446). All experiments involving live SARS-CoV-2 and MERS-CoV followed the approved standard operating procedures of the biosafety level 3 facility at the University of Hong Kong.

Culture of hEPSCs

Human EPSC cells were maintained on STO feeder layers and enzymatically passaged (1:10) every 3-5 days by briefly washing with PBS followed by treatment with TrypLE (Gibco, Catalog #12605036) for 5 minutes. Cells were dissociated and centrifuged (300 g for 3 minutes) in 10% fetal bovine serum (FBS)-containing medium (M10 medium). After removing supernatant, human EPSCs were resuspended and seeded in hEPSC Medium (EPSCM) supplemented with 5.0 μM Y27632 (Tocris Bioscience, Catalog #1254). hEPSCM is a N2B27-based media supplement with small molecular as in previous studies (Gao, X. et al. (2019) Nature Cell Biology 21(6), pages 687-699). N2B27 basal media contains 1-part DMEM/F12 (ThermoFisher Scientific, Catalog #21331020) and 1-part Neurobasal Medium (ThermoFisher Scientific, Catalog #21103049), 200×N2 supplement, and 100×B27 supplement.

Differentiation of hEPSCs to Trophoblast Lineages by the TGF-β Inhibitor SB431542

Human EPSCs were dissociated with TrypLE (Gibco, Catalog #12605036) and seeded in 100× Geltrex (ThermoFisher Scientific, Catalog #A1413302) coated six-well plates at a density of 1×10⁵ cells per well. Cells were cultured (pre-treatment) in 20% Knock-out Serum Replacement (KSR) media supplemented with 10 μM Y27632 for one day. From the second day, 10 μM SB431542 (Tocris Bioscience, Catalog #1614) was added into 20% KSR media to start the differentiation process. Cells were collected at the indicated time points for analysis.

Derivation of Human Trophoblast Stem Cell (hTSCs) from hEPSCs

Single cell-dissociated hEPSCs were plated on 6-well plates pre-coated with 100× Geltrex (ThermoFisher Scientific, Catalog #A1413302) at a density of 2,000 cells per well and cultured in Human Trophoblast-induced Stem Cell (hTSC) media. The hTSC media contained DMEM/F12 (Gibco, Catalog #21331-020) supplemented with 50.0 μM β-mercaptoethanol (ThermoFisher Scientific, Catalog #31350010), 0.2% FBS (Gibco, Catalog #10270), 0.5% Penicillin-Streptomycin-Glutamine (ThermoFisher Scientific, Catalog #10378016), 0.3% BSA (Bovine Serum Albumin, Gibco, Catalog #15260037), 1.0% ITS-X supplement (Gibco, Catalog #51500056), 50.0 μg/mL 2-Phospho-L-ascorbic acid trisodium salt (a Vitamin C derivative, Sigma-Aldrich, Catalog #49752-100G), 50.0 ng/mL Epidermal Growth Factor (EGF) (ThermoFisher Scientific, #PHG0311), 2.0 μM CHIR99021 (Glycogen synthase kinase 3 (GSK-3) inhibitor, Tocris Bioscience, Catalog #4423), 0.5 μM A83-01 (ALK-5 inhibitor, Tocris Bioscience, Catalog #2939), 1.0 μM SB431542 (Tocris Bioscience, Catalog #1614), 10.0 μM Valproic acid (VPA, StemCell Technologies, Catalog #72292) and 5.0 μM Y27632 (Tocris Bioscience, Catalog #1254). After 12-14 days of culture, the colonies with TSC-like morphologies were selected, dissociated in TrypLE (Gibco, Catalog #12605036), and re-plated on a plate pre-coated with 100×Geltrex. After 4-5 passages, the cells were collected for differentiation of syncytiotrophoblasts (STB) and extravillous trophoblasts (EVT) as described in previous studies (Okae, H. et al. (2018) Cell Stem Cell 22(1), pages 50-63.e6).

Differentiation of EPSC-TSCs to STBs

Wells of a six-well plate were coated with 100×Matrigel (Corning, Catalog #354230) for at least 1 hour. 1.0×10⁵ hTSCs were seeded per well in 2 mL STB medium. The STB medium contained DMEM/F12 (ThermoFisher Scientific, Catalog #21331020), supplemented with 50 μM β-mercaptoethanol, 0.5% Penicillin-Streptomycin-Glutamine (ThermoFisher Scientific, Catalog #10378016), 0.3% BSA, 1% ITS-X, 2.5 μM Y-27632, 2 μM Forskolin (Sigma-Aldrich, Catalog #F3917), and 4% KnockOut Serum Replacement (ThermoFisher Scientific, Catalog #10828028). The media was changed on day 3, and the cells were ready for downstream analysis on day 6.

Differentiation of EPSC-TSCs to EVTs

For EVT differentiation, wells of a 6-well plate were coated with 100×Matrigel (Corning, Catalog #354230) for at least 1 hour. 1.0×10⁵ hTSCs were seeded per well in 3.0 mL EVT basal medium. The EVT basal medium contained DMEM/F-12 (ThermoFisher Scientific, Catalog #21331020) supplemented with 50 μM β-mercaptoethanol, Penicillin-Streptomycin-Glutamine (ThermoFisher Scientific, Catalog #10378016), 0.3% BSA, 1% ITS-X, 7.5 μM A83-01, 2.5 μM Y27632, further supplemented with 4% KSR, 100 ng/mL NRG1 (Human Neuregulin-1, Cell Signaling, Catalog #5218SC) and 2% Matrigel. On day 3, the media was replaced with 2 mL EVT basal medium supplemented with 4% KSR and 0.5% Matrigel. On day 6, the media was replaced with 2 mL EVT basal medium, and Matrigel was added to a 0.5% final concentration. On day 8, the cells were ready for downstream analysis.

Generation of Trophoblast Organoids from EPSC-TSCs

EPSC-TSCs were digested with TrypLE (Gibco, Catalog #12605036) and dissociated to single cells via pipetting. After centrifugation, growth factor-reduced Matrigel (GFR-M, Corning) was added to reach a final concentration of 60% and the remaining 40% was trophoblast organoid medium (TOM). Matrigel/TOM (40 μL) containing 1.0×10⁵ hTSCs was seeded into the well center of a 24-well plate. The plates were incubated for 3 minutes at 37° C. in a CO₂ incubator. Next, the plates were turned upside down to ensure equal spreading of the cells in the well and incubated for 15 minutes at 37° C. in an incubator. Then, the solidifying GFR-M formed domes and were carefully overlaid with 500 μL TOM. Trophoblast organoids were allowed to form for 4-6 days at P0. Following 20 days in cell culture, trophoblast organoids were collected by dissolving Matrigel with recovery solution (Corning, Catalog #354253) and infected with the SARS-CoV-2 virus. TOM contained N2B27 basal media, 50 ng/mL recombinant human EGF, 1.5 μM CHIR99021, 80 ng/mL recombinant human R-spondin-1, 100 ng/mL recombinant human FGF-2, 50 ng/mL recombinant human HGF, 500 nM A83-01, 2.5 μM Prostaglandin E2, and 2 μM Y-27632. The TOM media was stored at 4° C. for up to 2 weeks.

Normally Used Cell Lines for SARS-CoV-2 Infection

Human colon epithelial (Caco-2) cells and African green monkey kidney (Vero E6) cells which are often used for virus infection were purchased from ATCC. All cells cultured in this study were maintained at 37° C. with 5% CO₂, unless stated otherwise, and had been confirmed to be free of mycoplasma contamination by PlasmoTest™ (InVivoGen).

SARS-CoV-2 Infection and Detection

The SARS-CoV-2 strain (SARS-CoV-2 HKU-001a; GenBank accession number MT230940) was isolated from a nasopharyngeal aspirate specimen from a COVID-19 patient in Hong Kong. SARS-CoV-2 stock was propagated using Vero E6 cells, and the titer of supernatant was assessed by plaque assays. All experiments involving with live SARS-CoV-2 followed the approved standard operating procedures of Biosafety Level 3 facilities in Queen Mary Hospital, The University of Hong Kong.

Indicated hEPSCs were seeded one day before infection and other types of differentiated cells at the suitable time point. Typically, ideal eSTBs are obtained from day 2 of hTSC differentiation. On the day of infection, cells were washed with PBS and infected at the indicated multiplicity of infection (MOI) by diluting viruses in basal medium. Cells were incubated at 37° C. for 2 hours. Subsequently, the inoculum was removed, replaced with complete culture medium, and further incubated until harvest. Cytopathic effects (CPE) were monitored daily via light microscopy, and cell supernatant and lysates at indicated time points were collected for RT-qPCR to assess the viral RNA load. Plaque assays were used to calibrate the viral RNA load to viral load determination.

Detection of SARS-CoV-2 Virus

For viral detection, the supernatants of the cultured cells that were challenged by SARS-CoV-2 were collected at various time points. Briefly, 560 μL of AVL buffer was added to a total volume of 140 μL of cell culture supernatant. Then, total RNA was extracted using the QIAamp® Viral RNA Mini Kit (Qiagen, Catalog #52906).

For virus replication kinetics assays, the extracted RNA was quantified with the one-step QuantiNova Probe RT-PCR kit (Qiagen, Catalog #208354). Each 20 μL reaction mixture contained 10 μL of 2×QuantiNova Probe RT-PCR Master Mix, 0.2 μL of QuantiNova Probe RT-Mix, 1.6 μL each of a 10 μM forward and reverse primer, 0.4 μL of 10 μM probe, 5 μL of the extracted RNA as template, and 1.2 μL of RNase-free water. Amplification was performed by incubating the reactions at 45° C. for 10 minutes for reverse transcription, 95° C. for 5 minutes for denaturation, 45 cycles of 95° C. for 5 seconds and 55° C. for 30 seconds, followed by a cooling step at 40° C. for 30 seconds. The primers and probe sequences were designed to detect the RNA-dependent RNA polymerase/helicase (RdRP/Hel) gene region of the SARS-CoV-2 virus. The primer sequences are listed in Tables 1A and 1B.

TABLE 1A
Sequences for Forward PCR Primers

| GENE NAME | FORWARD PRIMER (5′-3′) | SEQ ID NO: |
|---|---|---|
| SARS-CoV-2 | CGCATACAGTCTTRCAGGCT | 1 |
| ACE2 (Exon17-18) | GGAGTTGTGATGGGAGTGAT | 2 |
| ACE2 (Exon9-10) | TCCATTGGTCTTCTGTCACCCG | 3 |
| TMPRSS2 | CTCTACGGACCAAACTTCATC | 4 |
| CD147 | GGCTGTGAAGTCGTCAGAACAC | 5 |
| NANOG | TGAACCTCAGCTACAAACAG | 6 |
| OCT4 | CCTCACTTCACTGCACTGTA | 7 |
| SOX2 | TTCACATGTCCCAGCACTACCAGA | 8 |
| CDX2 | TTCACTACAGTCGCTACATCACC | 9 |
| GATA3 | ACATCTCGCCCTTCAGCCAC | 10 |
| KRT7 | AGGATGTGGATGCTGCCTAC | 11 |
| TEAD4 | CAGGTGGTGGAGAAAGTTGAGA | 12 |
| TFAP2C | ACAGGATCCATGTTGTGGAAAATAACCGAT | 13 |
| TP63 | AGAAACGAAGATCCCCAGATGA | 14 |
| CGB | ACCCTGGCTGTGGAGAAGG | 15 |
| ERVW-1 | GTTAATGACATCAAAGGCACCC | 16 |
| SDC1 | GCTGACCTTCACACTCCCCA | 17 |
| HLA-G | CAGATACCTGGAGAACGGGA | 18 |
| MMP2 | TGGCACCCATTTACACCTACAC | 19 |
| ITGB6 | CTCAACACAATAAAGGAGCTGGG | 20 |
| GABRP | TTTCTCAGGCCCAATTTTGGT | 21 |
| MUC16 | GGAGCACACGCTAGTTCAGAA | 22 |
| VTCN1 | TCTGGGCATCCCAAGTTGAC | 23 |
| cGAS | TAACCCTGGCTTTGGAATCAAAA | 24 |
| ZBP1 | TGGTCATCGCCCAAGCACTG | 25 |
| MDA5 | GAGCAACTTCTTTCAACCACAG | 26 |
| STING1 | AGCATTACAACAACCTGCTACG | 27 |
| IFNA2 | CTTGAAGGACAGACATGACTTTGGA | 28 |
| IFNB1 | AAACTCATGAGCAGTCTGCA | 29 |
| IFNG | TGGCTTTTCAGCTCTGCATC | 30 |
| IFNL1 | CGCCTTGGAAGAGTCACTCA | 31 |
| IFNL2 | AGTTCCGGGCCTGTATCCAG | 32 |
| IFNL3 | TCGCTTCTGCTGAAGGACTGCA | 33 |
| IFNL4 | ATGCGGCCGAGTGTCTGG | 34 |
| IL6 | GTCAGGGGTGGTTATTGCAT | 35 |
| IL28A | TCCAGTCACGGTCAGCA | 36 |
| TNF | CTCTTCTGCCTGCTGCACTTTG | 37 |
| HLA-A | CGAGGATGGCCGTCATGGCG | 38 |
| HLA-B | CAGTTCGTGAGGTTCGACAG | 39 |
| ITGA1 | CTGGACATAGTCATAGTGCTGGA | 40 |
| ITGA5 | GTCGGGGGCTTCAACTTAGAC | 41 |
| ITGA6 | CACATCTCCTCCCTGAGCAC | 42 |
| CDX2 | TTCACTACAGTCGCTACATCACC | 43 |
| ELF5 | TGCCCTCACGGTAATGTTGGA | 44 |
| GAPDH | CAAATTCCATGGCACCGTCA | 45 |
| human miR-103a | GTAGCAGCATTGTACAGGG | 46 |
| human miR-526b-3p | GTTTGGGAAAGTGCTTCCTTTT | 47 |
| human miR-517a | GTTTGGATCGTGCATCCTTTTA | 48 |
| human miR-517b | GTGCCTCTAGATGGAAGCA | 49 |
| human miR-525-3p | GTTGAAGGCGCTTCCCTTT | 50 |

TABLE 1B
Sequences for Reverse PCR Primers

| GENE NAME | REVERSE PRIMER (5′-3′) | SEQ ID NO: |
|---|---|---|
| SARS-CoV-2 | GTGTGATGTTGAWATGACATGGTC | 51 |
| ACE2 (Exon17-18) | GATGGAGGCATAAGGATTTT | 52 |
| ACE2 (Exon9-10) | AGACCATCCACCTCCACTTCTC | 53 |
| TMPRSS2 | CCACTATTCCTTGGCTAGAGTA | 54 |
| CD147 | ACCTGCTCTCGGAGCCGTTCA | 55 |
| NANOG | TGGTGGTAGGAAGAGTAAAG | 56 |
| OCT4 | CAGGTTTTCTTTCCCTAGCT | 57 |
| SOX2 | TCACATGTGTGAGAGGGGCAGTGTGC | 58 |
| CDX2 | TTGATTTTCCTCTCCTTTGCTC | 59 |
| GATA3 | CATGGCGGTGACCATGCTGGA | 60 |
| KRT7 | CACCACAGATGTGTCGGAGA | 61 |
| TEAD4 | GTGCTTGAGCTTGTGGATGAAG | 62 |
| TFAP2C | ATACTCGAGTTTCCTGTGTTTCTCCATTTT | 63 |
| TP63 | CTGTTGCTGTTGCCTGTACGTT | 64 |
| CGB | ATGGACTCGAAGCGCACA | 65 |
| ERVW-1 | CCCCATCTCAACAGGAAAACC | 66 |
| SDC1 | CAAAGGTGAAGTCCTGCTCCC | 67 |
| HLA-G | CAGTATGATCTCCGCAGGGT | 68 |
| MMP2 | ATGTCAGGAGAGGCCCCATAGA | 69 |
| ITGB6 | AAAGGGGATACAGGTTTTTCCAC | 70 |
| GABRP | GCTGTCGGAGGTATATGGTGG | 71 |
| MUC16 | GGTCTCTATTGAGGGGAAGGT | 72 |
| VTCN1 | TCCGCCTTTTGATCTCCGATT | 73 |
| cGAS | TGGGTACAAGGTAAAATGGCTTT | 74 |
| ZBP1 | GGCGGTAAATCGTCCATGCT | 75 |
| MDA5 | CACTTCCTTCTGCCAAACTTG | 76 |
| STING1 | GTTGGGGTCAGCCATACTCAG | 77 |
| IFNA2 | GGATGGTTTCAGCCTTTTGGA | 78 |
| IFNB1 | AGGAGATCTTCAGTTTCGGAGG | 79 |
| IFNG | CCGCTACATCTGAATGACCTG | 80 |
| IFNL1 | GAAGCCTCAGGTCCCAATTC | 81 |
| IFNL2 | GAGCCGGTACAGCCAATGGT | 82 |
| IFNL3 | CCTCCAGAACCTTCAGCGTCAG | 83 |
| IFNL4 | GCTCCAGCGAGCGGTAGTG | 84 |
| IL6 | AGTGAGGAACAAGCCAGAGC | 85 |
| IL28A | CAGCCTCAGAGTGTTTCTTCT | 86 |
| TNF | ATGGGCTACAGGCTTGTCACTC | 87 |
| HLA-A | CACATTCCGTGTCTCCTGGTCCC | 88 |
| HLA-B | CAGCCGTACATGCTCTGGA | 89 |
| ITGA1 | ACCTGTGTCTGTTTAGGACCA | 90 |
| ITGA5 | CCTGGCTGGCTGGTATTAGC | 91 |
| ITGA6 | TATCTTGCCACCCATCCTTG | 92 |
| CDX2 | TTGATTTTCCTCTCCTTTGCTC | 93 |
| ELF5 | TGATGCTCAAAGGCAGGGTAG | 94 |
| GAPDH | ATCGCCCCACTTGATTTTGG | 95 |

Plaque Assay

Plaque assays were performed as described in previous studies (Yuan, S. et al. (2021) Nature 593(7859), pages 418-423). Briefly, Vero E6 cells were seeded at 300,000 cells per well in 12-well tissue culture plates on the day prior to conducting the assay. Following 24 hours of incubation, a serial dilution of the supernatant was added to the cell monolayer and the plates were further incubated for 1 hour at 37° C. in 5% CO₂. Next, unbound viral particles were removed via aspiration of the media and washed once with DMEM. Monolayers were overlaid with media containing 1% low melting point agarose (Cambrex Corporation, East Rutherford, NJ, USA) in DMEM, inverted, and incubated as described above for another 72 hours. The wells were then fixed with 10% formaldehyde (BDH, Merck, Darmstadt, Germany) overnight. The agarose plugs were removed, the monolayers were stained with 0.7% crystal violet (BDH, Merck), and the plaques were counted. The Plaque assay experiments were performed in triplicate.

Guide RNA Design, RNA Synthesis, and Plasmid DNA Preparation

The human ACE2 exon 2 sequence was analyzed using the online CRISPOR tool to design a pair of highly specific gRNAs. Chemically synthesized ssDNA oligos were incubated at 95° C. for 10 minutes for annealing into dsDNAs, which were then ligated into a linearized empty gRNA vector using the DNA Ligation Kit, Mighty Mix (TaKaRa Bio, Catalog #6023). The ligation product was transformed into Chemically Competent DH 5a Cells (KT Health) and spread onto a lysogeny broth (LB) agar plate infused with ampicillin for selection. The following day, single colonies were picked and cultured in LB broth with ampicillin for plasmid miniprep using the TIANprep Rapid Mini Plasmid Kit (TianGen, Catalog #DP105). The ligated gRNA sequences were confirmed by Sanger Sequencing. The gRNA plasmids with the correct target sequences were amplified and extracted using the Endofree Plasmid Kit II (TianGen, Catalog #DP118) to purify a large amount of endotoxin-free gRNA plasmid DNA for electroporation. Plasmid DNA of BSD-Cas9 was similarly prepared as described in previous studies (Gao, X. et al. (2019) Nature Cell Biology 21(6), pages 687-699).

Electroporation and Selection

The hEPSCs were cultured on hexagonal Boron Nitride (hBN) feeders. Electroporation was performed when the cells reached 70% to 80% confluence. The plasmid DNA included Cas9 and double sgRNA in a ratio of 2:1:1 i.e., 2 parts of 4 μg Cas9, 1 part of 2 μg sgRNA1, and 1 part of 2 μg sgRNA2 per one million cells as one group. The plasmid DNA was added into opti-MEM (Gibco, Catalog #31985062). The hEPSCs were washed twice with PBS, then dissociated into single cells using 0.05% Trypsin-EDTA. M10 medium (DMEM with 10% FBS) was added to neutralize trypsin. Single cell hEPSCs were re-suspended in Opti-MEM medium containing DNA mixture. Electroporation was performed via the Bio-Rad Gene Pulser Xcell Electroporation Systems using a 0.4 cm cuvette with 230 V, 500 uF. Following electroporation, cells were seeded with recovery medium (500 μl EPSCM+10% KSR+10 μM Y27632). Cells were incubated overnight and then switched to normal EPSC medium. One day following electroporation, cells were selected by 10 μg/mL Blasticidin S HCl (ThermoFisher Scientific, Catalog #A1113903). Two days after electroporation, cells were selected by 10 μg/mL Blasticidin S HCl and 1 μg/ml Puromycin Dihydrochloride (ThermoFisher Scientific, Catalog #A1113802) for another 2 days. After 7-10 days, single colonies were picked, expanded, and genotyped.

Genotyping and Sanger Sequencing

Single colonies were picked and digested into single cells in a 96-well plate by 0.05% trypsin-EDTA for 3-5 minutes. Half of these cells were transferred into a 48 well plate with EPSCM medium and SNL feeders for culturing. The other half cells of the same colony were for genotyping which was performed by using primers designed to amplify the targeted band as well as the wild types. The genotyping primers according to targeted ACE2 were as follows: Forward primer: 5′-GTGGCCTGGTCACTCTTAAC-3′ (SEQ ID NO:96), and Reverse primer: 5′-CAAATAAAGGCAGCTGCTGTG-3′ (SEQ ID NO:97). The mutant PCR band was gel-purified and confirmed by Sanger sequencing for 148 bp deletion.

Reverse Transcription-Quantitative Polymerase Chain Reaction (RT-qPCR)

Total RNA extraction was performed using the Rneasy Mini Kit (Qiagen, Catalog #74104) as per manufacturer's instructions. The isolated RNA was reverse transcribed into complementary (cDNA) using the FastKing gDNA Dispelling RT SuperMix (TianGen, Catalog #KR118) on a thermal cycler. Intracellular SARS-CoV-2 viral gene expression was confirmed using the PowerUp™ SYBR™ Green Master Mix (Applied Biosystems). The primer sequences used are listed in Tables 1A and 1B. All qPCR experiments were performed using The StepOnePlus™ Real-Time PCR System (Applied Biosystems). Viral RNA load and gene expression levels were normalized to GAPDH using the A Ct method. Data were analyzed using one- and two-tailed Student's t-test on GraphPad Prism (version 8).

Immunofluorescence Staining

The samples were fixed in 4% paraformaldehyde (Sigma-Aldrich, Catalog #P6148) at room temperature for 15 minutes, permeabilized with 0.3% Triton-X100 (Sigma-Aldrich, Catalog #T8787) for 30 minutes, and blocked for 3 hours with 10% donkey serum (Sigma-Aldrich, Catalog #D9663) and 1% BSA (Sigma-Aldrich, Catalog #A2153). Samples were incubated with primary antibodies overnight in a 4° C. cold room. Next, fluorescence-conjugated secondary antibodies were used to incubate the samples at room temperature for 1 hour. Following antibody staining, samples were counter-stained with 10 μg/ml DAPI (4′,6-diamidino-2-phenylindole, ThermoFisher Scientific, Catalog #62248) for 10 minutes to mark nuclei and samples were imaged using a fluorescence microscope.

Western Blotting

Proteins were separated on 7.5% polyacrylamide gels (Bio-Rad, Catalog #1610180) and transferred to PVDF membrane in a Bio-Rad transblot turbo system according to manufacturer's instructions. The following primary antibodies were used: rabbit ACE2 (1:500, Abclonal, Catalog #A4612), rabbit β-actin (1:5000, Ab-mart, Catalog #P30002M). Goat anti-rabbit IgG H&L (HRP) (1:10000, Abcam, Catalog #ab205718) was used as the secondary antibody. Images were developed and analyzed via the ChemiDoc Imaging System.

Flow Cytometry

hEPSCs were digested with 0.25% trypsin/EDTA for 2-3 minutes at 37° C. and dissociated to single cells by pipetting. The dissociated cells were filtered with a 40 μm nylon mesh (Corning, Catalog #352235) to remove clumps. The cells were centrifuged, fixed using Fixation Medium according to the manufacturers' instructions (BD Cytofix, Catalog #554655), washed, and stored at 4° C. in PBS supplemented with 0.1% NaN₃ (Sigma-Aldrich, Catalog #199931) and 5% FBS (Gibco, Catalog #10270) prior to analysis with flow cytometry. All the samples were analyzed using ACEA NovoCyte Quanteon using 488 nm (530/30 bandpass filter) and 561 nm (610/20 bandpass filter) channels to detect Fluorescein isothiocyanate (FITC) and exclude autofluorescence. A 405 nm (445/45 bandpass filter) channel was used to detect DAPI positive cells. Fluorescence-activated cell sorting (FACS) data were analyzed using FlowJo software.

Trans-Well Invasion Assay

The invasion ability of TSC derived EVTs were determined via cell invasion assay (Corning, Catalog #354480) according to manufacturers' instructions. Briefly, the invasion chambers were incubated with warm DMEM base medium at 37° C. for 1 hour. After rehydration, the medium was removed. ETSC-EVTs (1×10⁵ cells/well) was prepared in DMEM basal medium. The mixture was added to an invasion chamber and placed in a 24 well culture plate, with the chamber filled with DMEM containing 10% FBS. The cells were incubated in the chamber for 22 hours. The non-invading cells remain in the upper surface of the polycarbonate membrane in the chamber and the invading cells attach to the bottom surface of the polycarbonate membrane. The media from the top surface of the membrane was aspirated, and non-invasive or non-migratory cells on the top surface of the membrane were removed by scrubbing with a cotton bud. The invasive or migratory cells on the lower surface were stained with crystal violet for 15 minutes. Observations and imaging of the membrane were conducted under a light microscope.

Statistical Analysis and Reproducibility

Statistical analysis was performed with Microsoft Excel and GraphPad Prism (version 8). P values were calculated using unpaired Student's t-tests. The number of measurements, number of independent experiments, and the statistical tests used for each analysis performed are described in the brief description of the drawings. Experiments were repeated independently for reproducibility of results.

RNA-Sequencing Analysis

Adaptor sequences and low-quality 3′ end sequences were removed via Cutadapt 4.0. Processed reads were assembled and mapped to the Human hg38 genome using HISAT2. Gene annotation was done using the Ensembl gene annotation system. FeatureCounts was used to quantify gene expression. Genes with mean count number <5 were filtered. Annotations of transposable elements (TE) were made using RepeatMasker available from the University of California Santa Cruz (UCSC) Genome Browser. SQuIRE (Software for Quantifying Interspersed Repeat Expression) with “total” mode was used to quantify and determine locus-specificity of TE expression.

DESeq2 was used to analyze differentially expressed genes (DEGs) and Tes. DEGs and Tes with expression fold change >1.5 and adjusted P-value<0.05 were significantly differentially expressed. Gene ontology (GO) and KEGG (Kyoto Encyclopedia of Genes and Genomes) analysis was performed using the R package “clusterProfiler”. GSEA software (Gene Set Enrichment Analysis) was performed using GSEApy and the gene sets were downloaded directly from the website: gsea-msigdb.org. The bigWig files for RNA-seq signals or coverage tracks were generated using bamCoverage from deepTools, and Integrative Genomics Viewer (IGV) was used for visualization of the genomic data. The sequences of HERV-W, HERV-FRD and HERV-K were mapped to the reference sequences provided in GenBank accession numbers AY101582.1, BC068585.1 and N675077.1 respectively and the reads were aligned using HISAT2. GenBank accession number MN985325 was used as the reference for mapping the SARS-CoV-2 sequences, the reads were aligned using HISAT2, and then quantified as described above.

scRNA Preprocessing

Preprocessing of scRNA sequences was performed according to the SCANPY pipelines (Single-Cell Analysis in Python, Wolf et al. (2018) Genome Biology 19(1), article number 15). Briefly, the normalization method TPM (Transcripts Per Million) was used to extract pre- to post-implantation cells from the dataset published by Zhou and colleagues (available at the GEO accession number: GSE109555; Zhou, F. et al. (2019) Nature 572(7771), pages 660-664), and placenta cells from the dataset published by Liu and colleagues (available at the GEO accession number: GSE89497; Liu, Y. et al. (2018) Cell Research 28(8), pages 819-832). In the published studies, cells were categorized according to their stages of differentiation and lineages into 7 coarse clusters (3 clusters in pre- to post-implantation, 3 clusters in the first-trimester placenta, and 1 cluster in the second-trimester placenta). The annotated dataset of Zhou and colleagues (Zhou, F. et al. (2019) Nature 572(7771), pages 660-664) was obtained from Castel et al. (Castel, G. et al. (2020) Cell Reports 33(8), article number 108419). Pseudotime analysis was performed on the sequence datasets from Zhou et al. (2019) and Liu et al. (2018) to map the sequences on to a series of one-dimensional quantities, called pseudotime. The combined dataset was pre-processed by (1) log-transforming of the TPM counts, (2) scaling to 10 on each gene, and (3) regressing out on the sequencing depth. The diffusion pseudotime (DPT) was calculated using scanpy.tl.dpt with default parameters as described in previous studies (Haghverdi, L. et al., (2016) Nature Methods 13(10), pages 845-848). Cells with inconsistent stage-lineage annotations and pseudotime values were removed, and 4041 and 952 cells were retained for downstream analysis, respectively. Pearson correlations were computed based on log counts for selected markers with ACE2 and visualized using scatter plots. Batch effects among bulk RNA-seq were also filtered prior to comparing the in-vitro cells to the in-vivo reference.

Integration of In Vitro and In Vivo Extraembryonic Datasets

For integrative analysis, the in vitro bulk RNA sequences were mapped to the in vivo scRNA reference using singular value decomposition (SVD) modeling (Halko, N., et al. (2011) Society for Industrial and Applied Mathematics, Vol. 53(2), pages 217-288); Pedregosa, F. et al. (2011) Journal of Machine Learning Research 12, pages 2825-2830). The assumption underlying the integrative analysis was that top components could capture cell identity and biological variations, regardless of sequencing types. First, the batch effects between the two scRNA datasets were filtered out to construct an extraembryonic landscape containing peri-implantation to placental stages. The SVD model was fitted using the whole transcriptome of the combined in vivo scRNA reference, and a 50-component decomposition result for the in vivo reference was generated. The fitted model was applied to project the in vitro cells (bulk RNA-seq) to the corresponding 50 components in the same space. The bulk RNA and scRNA datasets were concatenated as 50-component samples, (Uniform Manifold Approximation and Projection (UMAP) visualization was generated. As proof of effectivity of the SVD modelling in segregating cell types, for the in vivo sector, inter-cell type variations were captured by each cluster in the UMAP.

Whole-Transcriptome Correlation Analysis Between Bulk RNA-Sequencing of the In Vitro Cell Line and scRNA-Sequencing of the In Vivo Cell Line

Pseudo-bulk sets from the in vivo scRNA-sequences were generated with the assumption that bulk RNA-sequencing reflected additive effects of scRNA levels. For each in vivo subtype, two sets of 50 cells were randomly selected as two “pseudo-bulk cells” and an average was calculated to simulate two bulk samples for each subtype, thereby generating 14 pseudo-bulk samples. The 14 pseudo-bulk samples were merged with in vitro cells containing 19 samples of 10 types, and pair-wise Pearson coefficients were calculated across the whole transcriptome between each two samples. The coefficients were visualized in the cluster map to impute the corresponding in vivo stage of each in vitro cell line.

Integrative Pseudotime Analysis

Diffusion pseudotime (DBT) was computed for the whole in vivo reference using SCANPY (scanpy.tl.dpt, default parameters). The PI_TB to PI_STB subsets were extracted as the pseudotime reference set for downstream analysis of in vitro STB differentiated cells. There was 2,295 PI_TB cells and 1,282 PI_STB cells, constituting a unidirectional differentiation trajectory. As described above, a 50-component SVD model was fitted using the scRNA reference to reduce the dimensions of both scRNA and bulk RNA to a common 50-component space. It was hypothesized that these 50 components could predict the pseudotime calculated by SCANPY. The machine learning prediction process was conducted using sklearn.linear_model.LinearRegression (Zhao, C. et al. (2021) bioRxiv, doi:10.1101/2021.05.07.442980). Briefly, the 50 components and the pseudotimes of 1,721 PI_TB cells and 961 PI_STB cells (75% of each subtype) were used to train the linear regression model and the remaining 25% of each subtype were used as test sets to validate the model. The validation result was visualized using a density plot. The effectivity of the model was confirmed, and the model was applied to predict the pseudotime of in vitro differentiation cells. The theoretical “observed pseudotime” was calculated for in vitro cells using the following linear equation between the predicted and observed pseudotime in the in vivo reference:

Observed pseudotime=1.022×Predicted pseudotime−0.0032.

The pseudotime of each dataset was then merged to visualize the relative differentiation stage of each cell.

Quantification and Statistical Analysis

Statistical analysis was performed via GraphPad Prism (version 8) software using one- or two-tailed unpaired Student t-tests for comparisons between two groups and ANOVAs for comparisons between multiple groups. The threshold for statistical significance was p<0.05. Details on sample sizes, the number of replicates, statistical tests and p values for each experiment are provided in the relevant description of the drawings. Unless specified otherwise, “n” refers to the number of replicates.

### Results

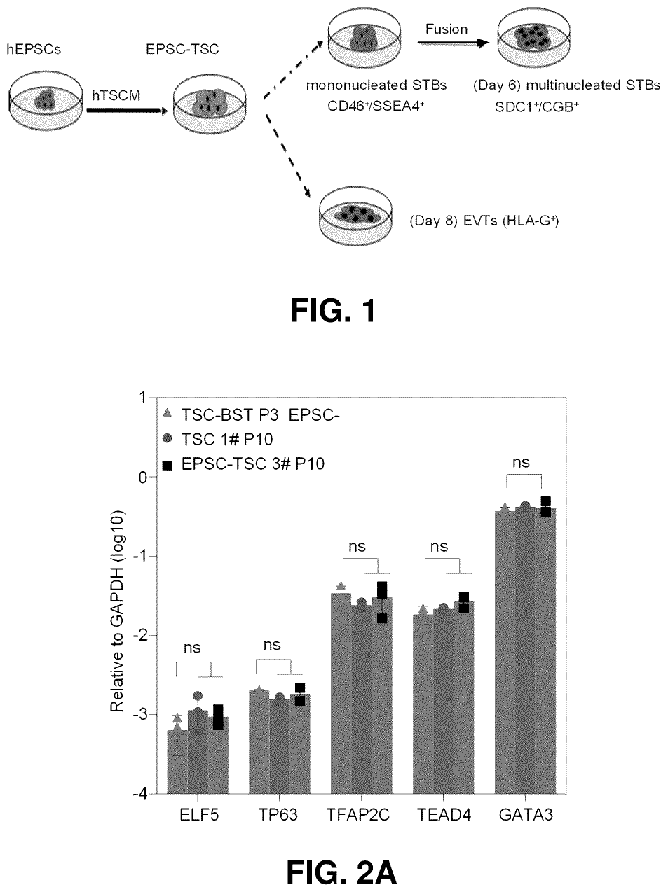

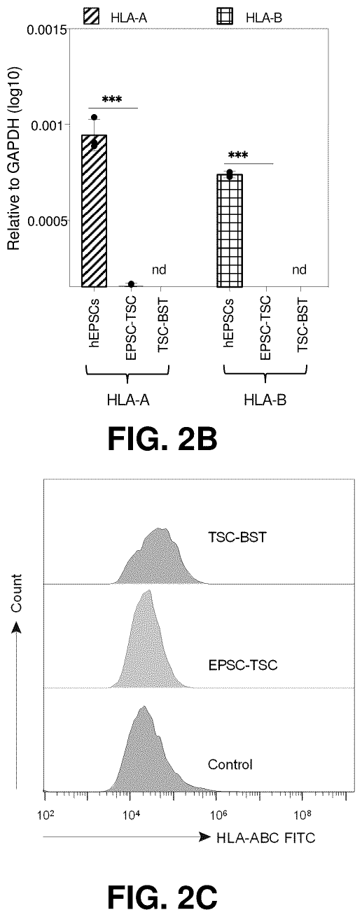

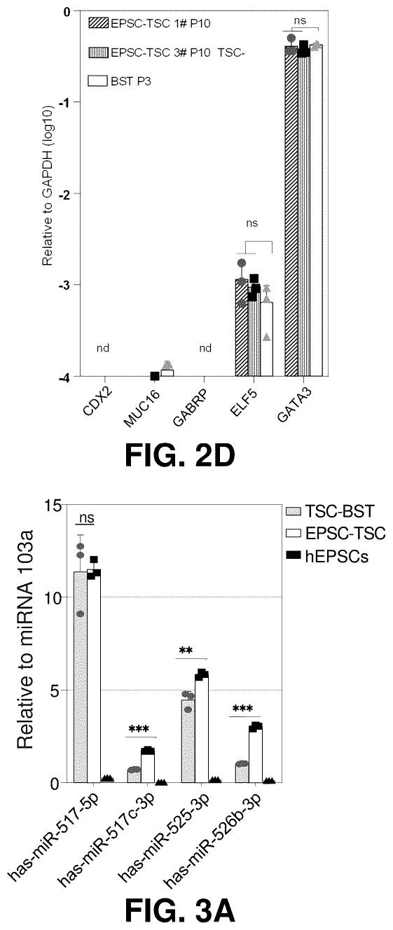

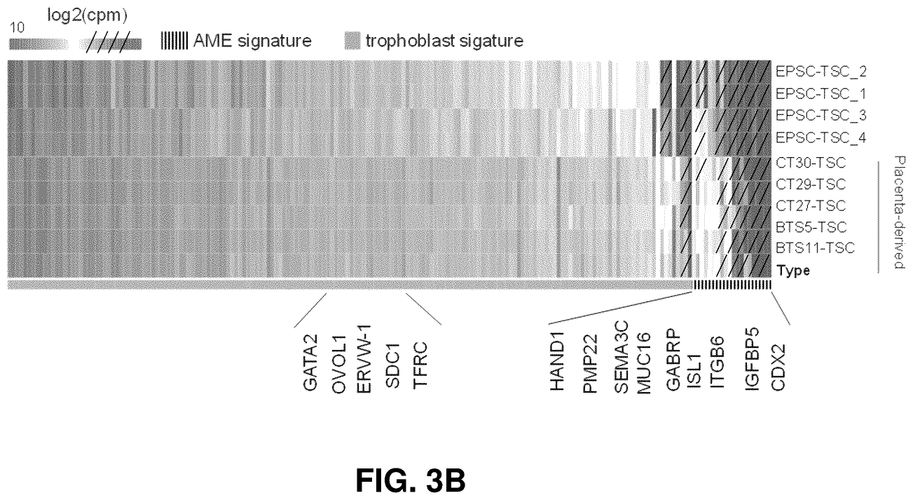

To develop hEPSCs as a model for studying COVID-19, several new human trophoblast stem cell (hTSC) lines were created based on previous studies with modifications (Okae, H. et al. (2018) Cell Stem Cell 22(1), pages 50-63. E6). The hEPSCs were generated from M1 hEPSCs, which were converted from primed human ESC line M1 using methods inspired by previous studies (Gao, X. et al. (2019) Nature Cell Biology 21(6), pages 687-699) and cultured as compact colonies with smooth colony edges in the medium containing three small-molecule inhibitors CHIR99021, A-419259 and XAV939 inhibiting glycogen synthase kinase 3β (GSK3β), SRC kinase family and Tankyrases, respectively (FIG. 1). The new EPSC-TSCs were confirmed via brightfield microscopy of hEPSCs and EPSC-TSCs EPSC-TSCs formed cobblestone-shaped colonies and expressed typical hTSC factors such as ELF5, TP63, TFAP2C, and GATA3, (FIG. 2A). They were low or negative for the classical HLA class I molecules HLA-A and HLA-B, like BST-TSC (FIGS. 2B and 2C). In both RNAseq analysis and individual gene RT-PCR, EPSC-TSCs did not express putative AME signature genes such as CDX2, MUC16, GABRP, ITGB6, or VTCN1 (Zheng, Y. et al., (2019) Nature 573(7774), pages 421-425) (FIG. 2D and Table 2), similar to those derived from human blastocyst and placenta cytotrophoblasts (BST-TSC, CT-TSC, CT27-TSC, CT29-TSC, CT30-TSC, BTS5-TSC and BTS-11-TSC) (Okae, H. et al. (2018) Cell Stem Cell 22(1), pages 50-63. E6; Sheridan, M. A. et al., (2021). Development 148(41): article dev199749). EPSC-TSCs also expressed the trophoblast-specific C19MC miRNAs including has-miR-517c-3p, 517-5p, 525-3p, and 526b-3p (Lee, C. Q., et al. (2016) Stem cell reports Vol. 6(2), pages 257-272; FIG. 3A), similar to human blastocyst-derived TSCs (BST-TSC) (Okae, H. et al. (2018) Cell Stem Cell 22(1), pages 50-63. E6).

The] transcriptomes of hTSCs derived from EPSCs were further compared with primed and naïve stem cells and of in vivo origins (Dong, C. et al. (2020) Elife Vol. 9, Article e52504; Gao, X. et al. (2019) Nature cell biology Vol. 21 (6), pages 687-699; Guo, G. et al. (2021). Cell Stem Cell 28(6), pages 1040-1056. E6; Okae, H. et al. (2018) Cell Stem Cell 22(1), pages 50-63. E6; Sheridan, M. A. et al., (2021). Development 148(41): article dev199749; Wei, Y. et al. (2021) Science Advances Vol. 7(33), Article eabf4416) (Table 3), and found that EPSC-TSC lines and naïve-TSCs were distinct from primed-TSCs (FIG. 3B).

TABLE 2
Amnion and Trophoblast Signature Genes

| GENE | TYPE | ANNOTATION |
|---|---|---|
| CA3 | Amnion | carbonic anhydrase 3 |
| DIRAS2 | Amnion | DIRAS family GTPase 2 |
| FXYD6 | Amnion | FXYD domain containing ion |
| | | transport regulator 6 |
| GABRP | Amnion | gamma-aminobutyric acid type |
| | | A receptor pi subunit |
| HEPH | Amnion | hephaestin |
| IGFBP5 | Amnion | insulin like growth factor |
| | | binding protein 5 |
| ISL1 | Amnion | ISL LIM homeobox 1 |
| LMO1 | Amnion | LIM domain only 1 |
| LRRN1 | Amnion | leucine rich repeat neuronal 1 |
| MSRB2 | Amnion | methionine sulfoxide reductase B2 |
| RARRES2 | Amnion | retinoic acid receptor responder 2 |
| SDC2 | Amnion | syndecan 2 |
| CDX2 | Amnion | caudal type homeobox 2 |
| MUC16 | Amnion | mucin 16, cell surface associated |
| ITGB6 | Amnion | integrin subunit beta 6 |
| HAND1 | Amnion | heart and neural crest derivatives |
| | | expressed 1 |
| SEMA3C | Amnion | semaphorin 3C |
| PMP22 | Amnion | peripheral myelin protein 22 |
| TRIM55 | Amnion | tripartite motif containing 55 |
| AC011453.1 | TE | NA |
| ADAM15 | TE | ADAM metallopeptidase domain 15 |
| AEN | TE | apoptosis enhancing nuclease |
| AGPAT5 | TE | 1-acylglycerol-3-phosphate |
| | | O-acyltransferase 5 |
| AKAP13 | TE | A-kinase anchoring protein 13 |
| AKAP8 | TE | A-kinase anchoring protein 8 |
| ATP13A3 | TE | ATPase 13A3 |
| ATXN2L | TE | ataxin 2 like |
| B4GALT1 | TE | beta-1,4-galactosyltransferase 1 |
| BAIAP2 | TE | BAI1 associated protein 2 |
| BOP1 | TE | block of proliferation 1 |
| BUB1 | TE | BUB1 mitotic checkpoint serine/ |
| | | threonine kinase |
| BYSL | TE | bystin like |
| C6orf106 | TE | chromosome 6 open reading frame 106 |
| CARS | TE | cysteinyl-tRNA synthetase |
| CCDC86 | TE | coiled-coil domain containing 86 |
| CCKBR | TE | cholecystokinin B receptor |
| CCNA2 | TE | cyclin A2 |
| CCND3 | TE | cyclin D3 |
| CCR7 | TE | C-C motif chemokine receptor 7 |
| CD3EAP | TE | CD3e molecule associated protein |
| CDC6 | TE | cell division cycle 6 |
| CDCA4 | TE | cell division cycle associated 4 |
| CDK12 | TE | cyclin dependent kinase 12 |
| CEP85 | TE | centrosomal protein 85 |
| CHERP | TE | calcium homeostasis endoplasmic |
| | | reticulum protein |
| CYP11A1 | TE | cytochrome P450 family 11 |
| | | subfamily A member 1 |
| CYP19A1 | TE | cytochrome P450 family 19 |
| | | subfamily A member 1 |
| DCAF12 | TE | DDB1 and CUL4 associated |
| | | factor 12 |
| DDX54 | TE | DEAD-box helicase 54 |
| DEPP1 | TE | NA |
| DHX38 | TE | DEAH-box helicase 38 |
| DLG5 | TE | discs large MAGUK scaffold |
| | | protein 5 |
| DLX3 | TE | distal-less homeobox 3 |
| DNAJA3 | TE | DnaJ heat shock protein |
| | | family (Hsp40) member A3 |
| DNM2 | TE | dynamin 2 |
| DNMT3B | TE | DNA methyltransferase 3 beta |
| DNMT3L | TE | DNA methyltransferase 3 like |
| DPH2 | TE | DPH2 homolog |
| DPPA3 | TE | developmental pluripotency |
| | | associated 3 |
| EAF1 | TE | ELL associated factor 1 |
| ECPAS | TE | NA |
| EHD1 | TE | EH domain containing 1 |
| EHD4 | TE | EH domain containing 4 |
| ELMSAN1 | TE | ELM2 and Myb/SANT domain |
| | | containing 1 |
| EP300 | TE | E1A binding protein p300 |
| ERVW-1 | TE | endogenous retrovirus group |
| | | W member 1, |
| | | envelope |
| FAM98A | TE | family with sequence similarity |
| | | 98 member A |
| FSCN1 | TE | fascin actin-bundling protein 1 |
| FURIN | TE | furin, paired basic amino acid |
| | | cleaving enzyme |
| FYB1 | TE | NA |
| GABARAPL1 | TE | GABA type A receptor associated |
| | | protein like 1 |
| GATA2 | TE | GATA binding protein 2 |
| GATAD2A | TE | GATA zinc finger domain |
| | | containing 2A |
| GCM1 | TE | glial cells missing homolog 1 |
| GCN1 | TE | GCN1, eIF2 alpha kinase activator |
| | | homolog |
| GDE1 | TE | glycerophosphodiester |
| | | phosphodiesterase 1 |
| GEMIN4 | TE | gem nuclear organelle associated |
| | | protein 4 |
| GJA5 | TE | gap junction protein alpha 5 |
| GNPNAT1 | TE | glucosamine-phosphate N- |
| | | acetyltransferase 1 |
| GREM2 | TE | gremlin 2, DAN family BMP |
| | | antagonist |
| GSE1 | TE | Gse1 coiled-coil protein |
| GTSF1 | TE | gametocyte specific factor 1 |
| GYS1 | TE | glycogen synthase 1 |
| HCFC1 | TE | host cell factor C1 |
| HK2 | TE | hexokinase 2 |
| HMOX1 | TE | heme oxygenase 1 |
| KNL1 | TE | kinetochore scaffold 1 |
| KNSTRN | TE | kinetochore localized astrin/ |
| | | SPAG5 binding protein |
| LASP1 | TE | LIM and SH3 protein 1 |
| LCMT1-AS2 | TE | NA |
| LETM1 | TE | leucine zipper and EF-hand |
| | | containing transmembrane protein 1 |
| LINC00668 | TE | NA |
| LMNB2 | TE | lamin B2 |
| LRRC58 | TE | leucine rich repeat containing 58 |
| LYAR | TE | Ly1 antibody reactive |
| MAK16 | TE | MAK16 homolog |
| MBD3 | TE | methyl-CpG binding domain protein 3 |
| MCM10 | TE | minichromosome maintenance 10 |
| | | replication initiation factor |
| MED12L | TE | mediator complex subunit 12 like |
| MED13 | TE | mediator complex subunit 13 |
| METTL13 | TE | methyltransferase like 13 |
| MFN2 | TE | mitofusin 2 |
| MIEF1 | TE | mitochondrial elongation factor 1 |
| MKI67 | TE | marker of proliferation Ki-67 |
| MRGPRX1 | TE | MAS related GPR family member X1 |
| MTHFD2 | TE | methylenetetrahydrofolate |
| | | dehydrogenase (NADP+ dependent) |
| | | 2, methenyltetrahydrofolate |
| | | cyclohydrolase |
| MTIF2 | TE | mitochondrial translational |
| | | initiation factor 2 |
| MYBBP1A | TE | MYB binding protein 1a |
| NABP1 | TE | nucleic acid binding protein 1 |
| NAT10 | TE | N-acetyltransferase 10 |
| NCLN | TE | nicalin |
| NCOA3 | TE | nuclear receptor coactivator 3 |
| NID1 | TE | nidogen 1 |
| NLRP2 | TE | NLR family pyrin domain containing 2 |
| NLRP7 | TE | NLR family pyrin domain containing 7 |
| NOL10 | TE | nucleolar protein 10 |
| NOL6 | TE | nucleolar protein 6 |
| NOP2 | TE | NOP2 nucleolar protein |
| NUP98 | TE | nucleoporin 98 |
| OGT | TE | O-linked N-acetylglucosamine |
| | | (GlcNAc) transferase |
| OVOL1 | TE | ovo like transcriptional repressor 1 |
| PFAS | TE | phosphoribosylformylglycinamidine |
| | | synthase |
| PGF | TE | placental growth factor |
| PIM1 | TE | Pim-1 proto-oncogene, serine/ |
| | | threonine kinase |
| PIP5K1A | TE | phosphatidylinositol-4-phosphate |
| | | 5-kinase |
| | | type 1 alpha |
| PKMYT1 | TE | protein kinase, membrane associated |
| | | tyrosine/threonine 1 |
| PMM2 | TE | phosphomannomutase 2 |
| PNP | TE | purine nucleoside phosphorylase |
| POLR3E | TE | RNA polymerase III subunit E |
| PPIF | TE | peptidylprolyl isomerase F |
| PPP1R10 | TE | protein phosphatase 1 regulatory |
| | | subunit 10 |
| PPRC1 | TE | peroxisome proliferator-activated |
| | | receptor gamma, coactivator-related 1 |
| PRPS2 | TE | phosphoribosyl pyrophosphate |
| | | synthetase 2 |
| PRRC2B | TE | proline rich coiled-coil 2B |
| PSME4 | TE | proteasome activator subunit 4 |
| PTDSS1 | TE | phosphatidylserine synthase 1 |
| PTGES | TE | prostaglandin E synthase |
| RAB11FIP4 | TE | RAB11 family interacting protein 4 |
| RAB35 | TE | RAB35, member RAS oncogene family |
| RACGAP1 | TE | Rac GTPase activating protein 1 |
| NLRP2 | TE | NLR family pyrin domain containing 2 |
| NLRP7 | TE | NLR family pyrin domain containing 7 |
| NOL10 | TE | nucleolar protein 10 |
| NOL6 | TE | nucleolar protein 6 |
| NOP2 | TE | NOP2 nucleolar protein |
| NUP98 | TE | nucleoporin 98 |
| OGT | TE | O-linked N-acetylglucosamine |
| | | (GlcNAc) transferase |
| OVOL1 | TE | ovo like transcriptional repressor 1 |
| PFAS | TE | phosphoribosylformylglycinamidine |
| | | synthase |
| PGF | TE | placental growth factor |
| PIM1 | TE | Pim-1 proto-oncogene, serine/ |
| | | threonine kinase |
| PIP5K1A | TE | phosphatidylinositol-4-phosphate |
| | | 5-kinase type 1 alpha |
| PKMYT1 | TE | protein kinase, membrane associated |
| | | tyrosine/threonine 1 |
| PMM2 | TE | phosphomannomutase 2 |
| PNP | TE | purine nucleoside phosphorylase |
| POLR3E | TE | RNA polymerase III subunit E |
| PPIF | TE | peptidylprolyl isomerase F |
| PPP1R10 | TE | protein phosphatase 1 regulatory |
| | | subunit 10 |
| PPRC1 | TE | peroxisome proliferator-activated |
| | | receptor |
| | | gamma, coactivator-related 1 |
| PRPS2 | TE | phosphoribosyl pyrophosphate |
| | | synthetase 2 |
| PRRC2B | TE | proline rich coiled-coil 2B |
| PSME4 | TE | proteasome activator subunit 4 |
| PTDSS1 | TE | phosphatidylserine synthase 1 |
| PTGES | TE | prostaglandin E synthase |
| RAB11FIP4 | TE | RAB11 family interacting protein 4 |
| RAB35 | TE | RAB35, member RAS oncogene family |
| RACGAP1 | TE | Rac GTPase activating protein 1 |
| RANGAP1 | TE | Ran GTPase activating protein 1 |
| RBM14 | TE | RNA binding motif protein 14 |
| RBM47 | TE | RNA binding motif protein 47 |
| RCC1 | TE | regulator of chromosome condensation 1 |
| RCL1 | TE | RNA terminal phosphate cyclase like 1 |
| RDH13 | TE | retinol dehydrogenase 13 |
| REEP1 | TE | receptor accessory protein 1 |
| RHOBTB1 | TE | Rho related BTB domain containing 1 |
| RHOG | TE | ras homolog family member G |
| RHOT2 | TE | ras homolog family member T2 |
| RNF168 | TE | ring finger protein 168 |
| RP2 | TE | RP2, ARL3 GTPase activating protein |
| RRM2 | TE | ribonucleotide reductase regulatory |
| | | subunit M2 |
| RRP12 | TE | ribosomal RNA processing 12 homolog |
| RRS1 | TE | ribosome biogenesis regulator homolog |
| RYBP | TE | RING1 and YY1 binding protein |
| S1PR2 | TE | sphingosine-1-phosphate receptor 2 |
| SDC1 | TE | syndecan 1 |
| SENP5 | TE | SUMO1/sentrin specific peptidase 5 |
| SESN2 | TE | sestrin 2 |
| SLC1A3 | TE | solute carrier family 1 member 3 |
| SLC1A5 | TE | solute carrier family 1 member 5 |
| SLC35F6 | TE | solute carrier family 35 member F6 |
| SLC40A1 | TE | solute carrier family 40 member 1 |
| SLC4A2 | TE | solute carrier family 4 member 2 |
| SLC7A5 | TE | solute carrier family 7 member 5 |
| SLC7A6 | TE | solute carrier family 7 member 6 |
| SMG5 | TE | SMG5, nonsense mediated mRNA decay |
| | | factor |
| SNX8 | TE | sorting nexin 8 |
| SOAT1 | TE | sterol O-acyltransferase 1 |
| SP6 | TE | Sp6 transcription factor |
| SRCAP | TE | Snf2 related CREBBP activator protein |
| SRRT | TE | serrate, RNA effector molecule |
| ST6GAL1 | TE | ST6 beta-galactoside alpha-2,6- |
| | | sialyltransferase 1 |
| STS | TE | steroid sulfatase |
| SURF6 | TE | surfeit 6 |
| TEAD1 | TE | TEA domain transcription factor 1 |
| TFRC | TE | transferrin receptor |
| TGFBR3 | TE | transforming growth factor beta |
| | | receptor 3 |
| TIGAR | TE | TP53 induced glycolysis regulatory |
| | | phosphatase |
| TLE3 | TE | transducin like enhancer of split 3 |
| TMEM109 | TE | transmembrane protein 109 |
| TOB2 | TE | transducer of ERBB2, 2 |
| TRAF4 | TE | TNF receptor associated factor 4 |
| TRIP12 | TE | thyroid hormone receptor interactor 12 |
| TRIP13 | TE | thyroid hormone receptor interactor 13 |
| TYRO3 | TE | TYRO3 protein tyrosine kinase |
| UBR4 | TE | ubiquitin protein ligase E3 component |
| | | n-recognin 4 |
| USP5 | TE | ubiquitin specific peptidase 5 |
| UTP20 | TE | UTP20, small subunit processome |
| | | component |
| VARS | TE | valyl-tRNA synthetase |
| ZFHX3 | TE | zinc finger homeobox 3 |
| TFRC | TE | transferrin receptor |
| TGFBR3 | TE | transforming growth factor beta |
| | | receptor 3 |
| TIGAR | TE | TP53 induced glycolysis regulatory |
| | | phosphatase |
| TLE3 | TE | transducin like enhancer of split 3 |
| TMEM109 | TE | transmembrane protein 109 |
| TOB2 | TE | transducer of ERBB2, 2 |
| TRAF4 | TE | TNF receptor associated factor 4 |
| TRIP12 | TE | thyroid hormone receptor interactor 12 |
| TRIP13 | TE | thyroid hormone receptor interactor 13 |
| TYRO3 | TE | TYRO3 protein tyrosine kinase |
| UBR4 | TE | ubiquitin protein ligase E3 component |
| | | n-recognin 4 |
| USP5 | TE | ubiquitin specific peptidase 5 |
| UTP20 | TE | UTP20, small subunit processome component |
| VARS | TE | valyl-tRNA synthetase |
| ZFHX3 | TE | zinc finger homeobox 3 |
| UBR4 | TE | ubiquitin protein ligase E3 component |
| | | n-recognin 4 |

TABLE 3
Culture conditions for hTSCs

| | CO₂ | | TSCM | TSCM | TSCM | TSCM | |
|---|---|---|---|---|---|---|---|
| Cell | and | | modification | modification | modification | modification | |
| Type | O₂ | ECM | 1 | 2 | 3 | 4 | REF |
| TSC- | 5% CO₂ | Collagen | 0.5 uM | 1 uM | 1.5 ug/ml | 0.8 mM | 1 |
| BST/ct | | IV | A83-01 | SB431542 | L-ascorbic | VPA | |
| | | | | | acid | | |
| Primed- | 5% CO₂ | Matrigel | 0.5 uM | 1 uM | 1.5 ug/ml | 0.8 mM | 2 |
| TSC | | | A83-01 | SB431542 | L-ascorbic | VPA | |
| | | | | | acid | | |
| Naïve- | 7% CO₂ | Collagen | 1.0 uM | Without | 1.5 mg/ml L- | 0.8 mM | 3 |
| TSC | and 5% | IV/MEF | A83-01 | SB431542 | ascorbic acid | VPA | |
| (PXGL) | O2 | | | | | | |
| Naïve- | 5% CO₂ | Collagen | 0.5 uM | 1 uM | 1.5 ug/ml | 0.8 mM | 4 |
| TSC | and 20% | IV | A83-01 | SB431542 | L-ascorbic | VPA | |
| (5iLA) | O2 | | | | acid | | |
| EPSC- | 5% CO₂ | Geltrex | 0.5 uM | 1 uM | 50 ug/ml | 10 uM | 5 |
| TSC | | | A83-01 | SB431542 | | VPA | |

REF1: Okae, H., et al. (2018)
REF2: Wei, Y., et al. (2021)
REF3: Guo, G. et al. (2021)
REF4: Dong, C et al. (2020)
REF5: This Study

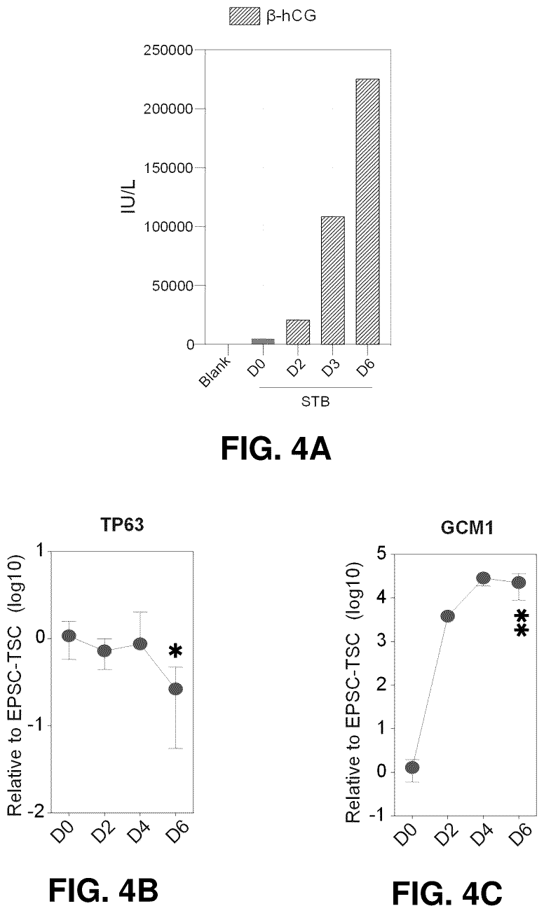

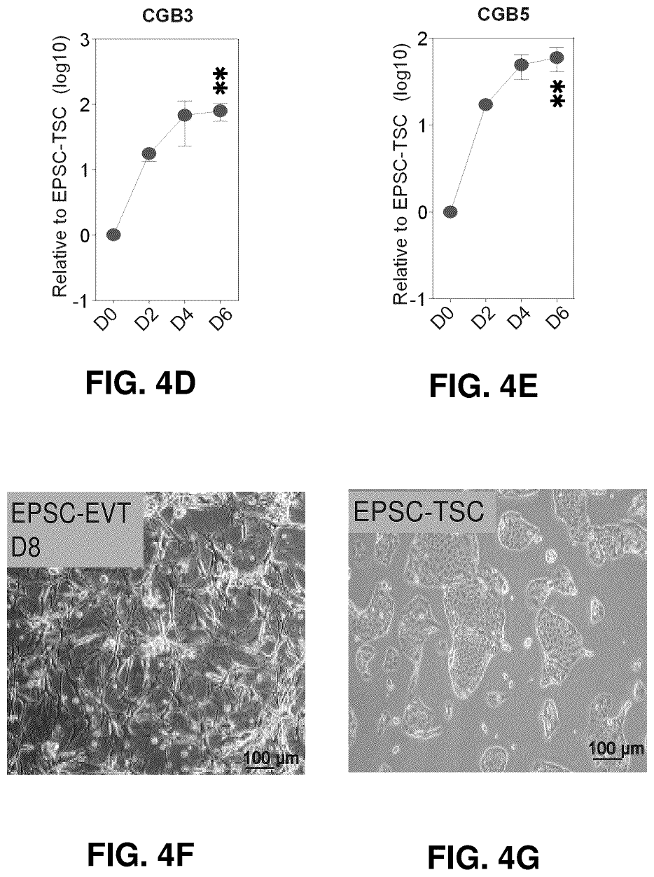

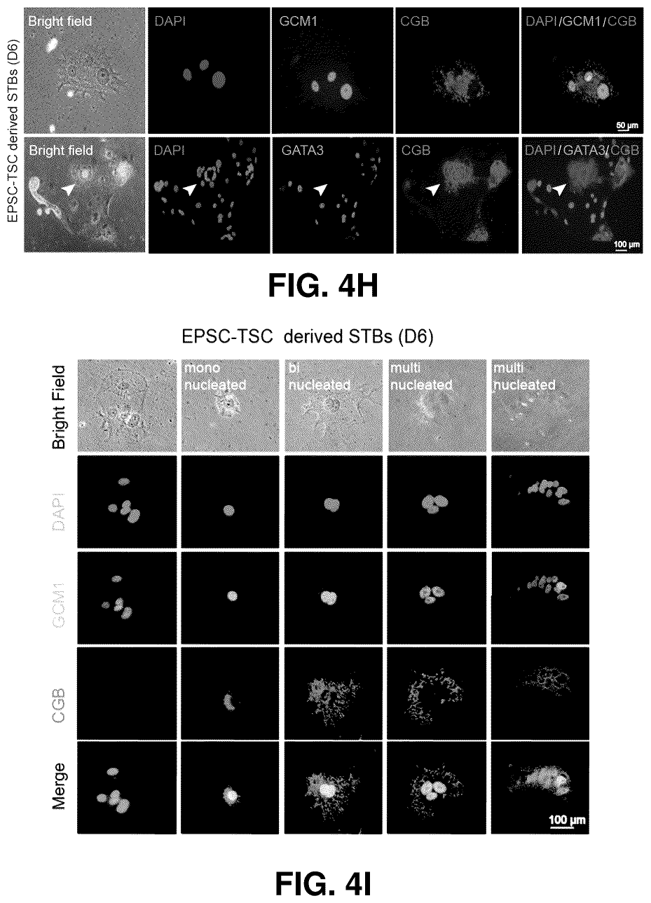

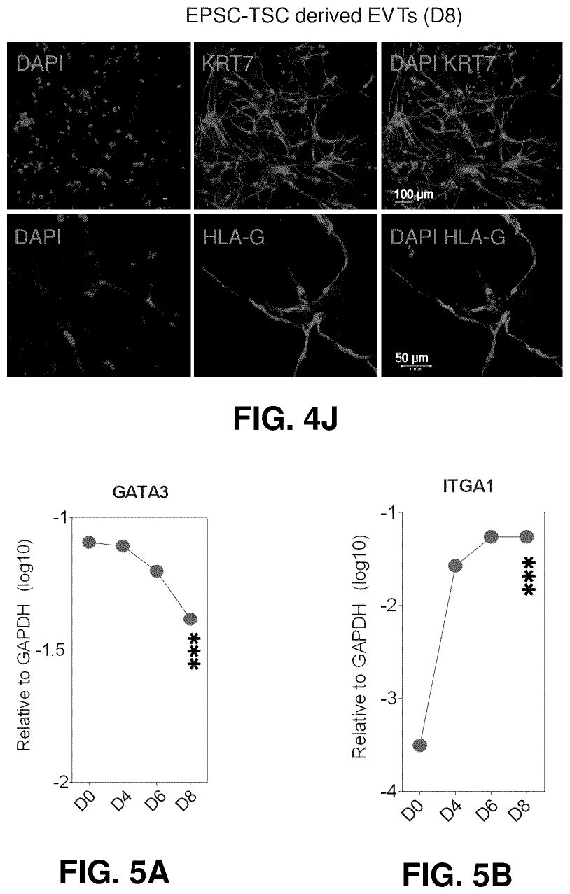

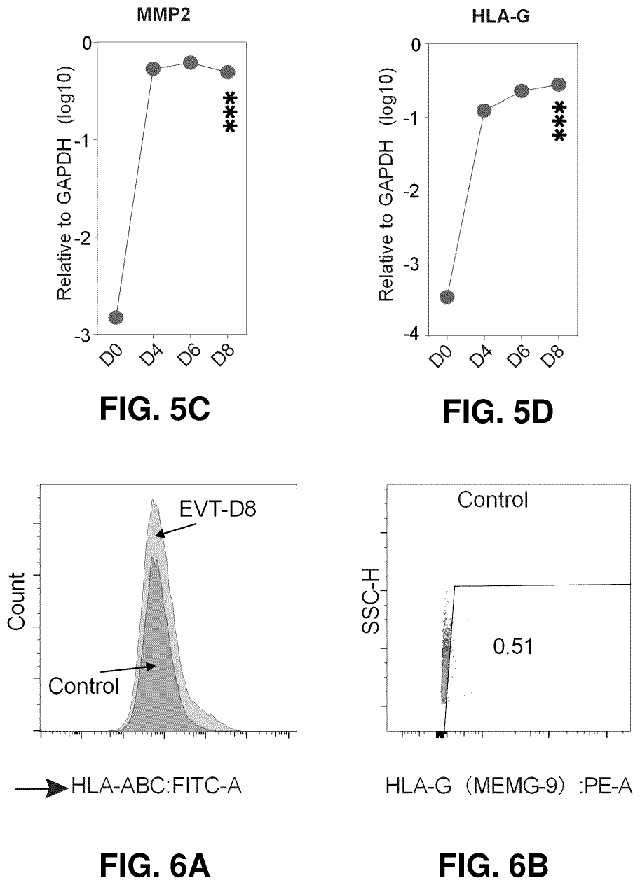

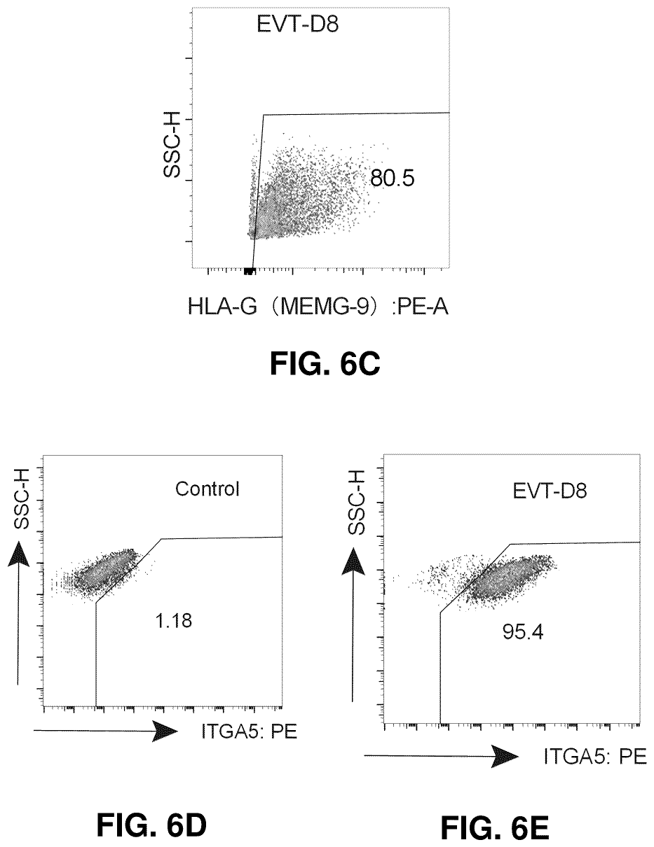

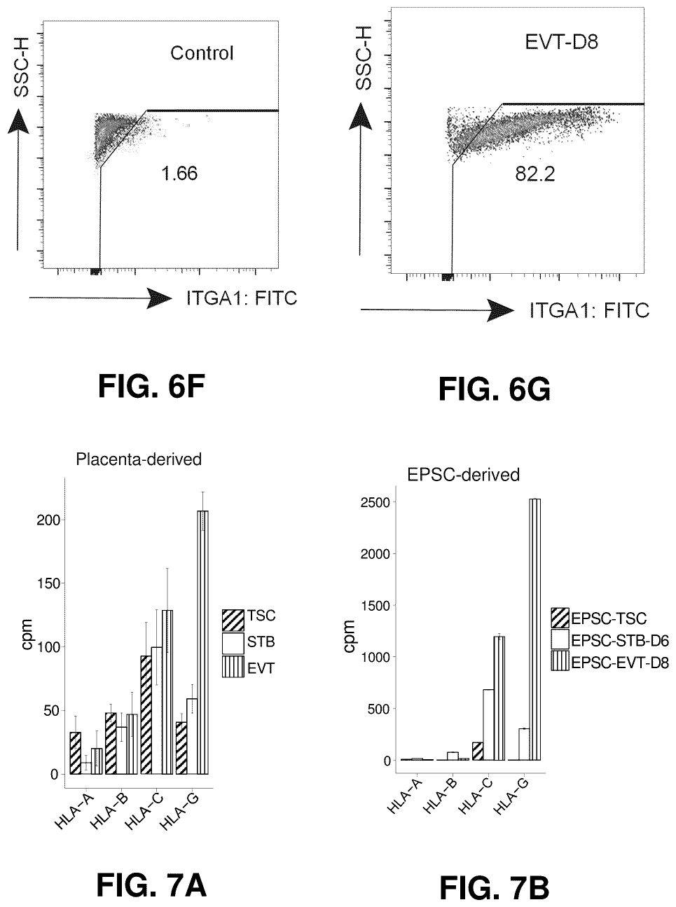

EPSC-TSCs were induced to generate STBs (FIGS. 4A, 4F, and 4G). Trophoblast progenitor transcription factor gene TP63 was reduced whereas STB genes such as GCM1, β chorionic gonadotrophin 3 gene (CGB3) and CGB5 quickly increased (FIGS. 4B-4E). Immunofluorescence staining of day 6 differentiated cells (STB-D6) detected GCM1⁺ and CGB⁺ and multinucleated STBs (FIGS. 4H and 4I), which appeared to have lost GATA3 expression (FIG. 4H). Functionally, properly folded, and secreted β-hCG hormone were detected in the supernatant of STB-D6 cells via ELISA tests (FIG. 4A). Under TGFβ inhibition, EPSC-TSCs efficiently generated EVTs, which had the typical EVT morphology and were stained positively for KRT7, HLA-G, ITGA1 and IGTA5 (FIG. 4J). The EVTs are cells with extensive cytoplasm, which contains necessary organelles for endocrine functions. EVT genes ITGA1, MMP2 and HLA-G were rapidly increased whereas GATA3 was reduced (FIGS. 5A-5D). Some HLA-G+ EVTs were GATA3. On day 8, most cells were positive for HLA-G (80%) (FIGS. 6B and 6C), ITGA1 (82.2%) (FIGS. 6F and 6G) and ITGA5 (95.4%) (FIGS. 6D and 6E), but negative for the classical HLA class I molecules, HLA-A and -B (FIG. 6A). Functionally, EPSC-TSC derived EVTs possessed potent invasiveness capability.

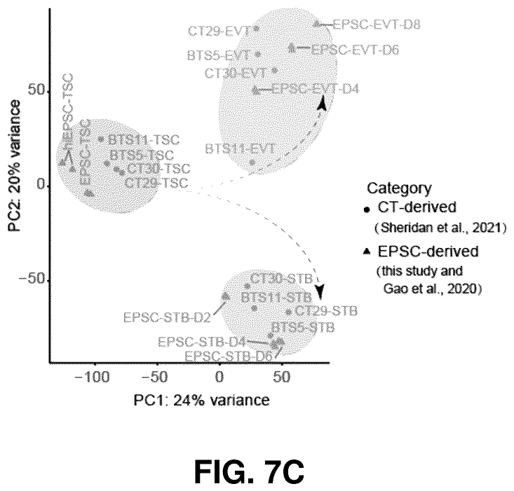

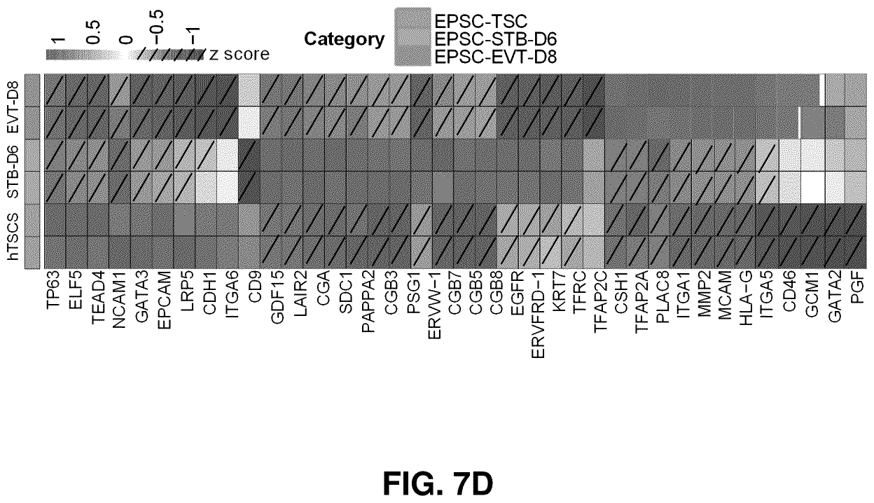

RNAseq analysis was performed on the EPSC-TSCs and their derivative STBs (D1, D2, D4 and D6) and EVTs ((D4, D6 and D8). Each set of cells were compared to human primary trophoblast-derived hTSCs (CT-TSC, BTS-TSC) and their derivatives, STB and EVT (Sheridan, M. A. et al., (2021). Development 148(41): article dev199749) (FIG. 7D). Principal component analysis (PCA) showed that EPSC-TSCs again clustered close to both CT-TSC and BTS-TSC, while EPSC-EVTs cluster most closely with CT-EVTs/BTS-EVTs and EPSC-STBs to CT-STBs/BTS-STBs (FIG. 7C). RNAseq analysis also confirmed low classical HLA class I molecules HLA-A and -B (FIG. 7B) in all TSCs as in those originated from the placenta trophoblasts (FIG. 7A) (Sheridan, M. A. et al., (2021). Development 148(41): article dev199749).

### Example 2: Trophoblasts Derived from hEPSCs Molecularly Resemble Those in the Human Peri-Implantation Embryos and the Placenta

Methods

The methods for Example 2 are described in Example 1.

Results

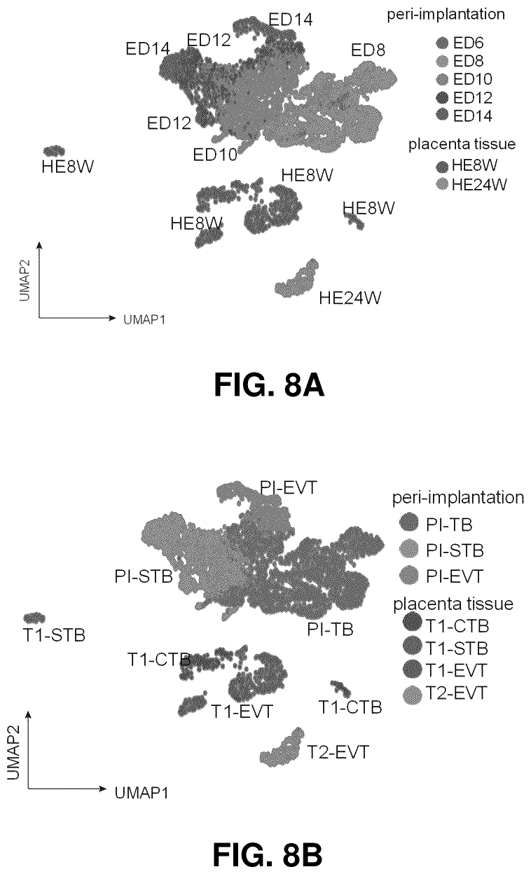

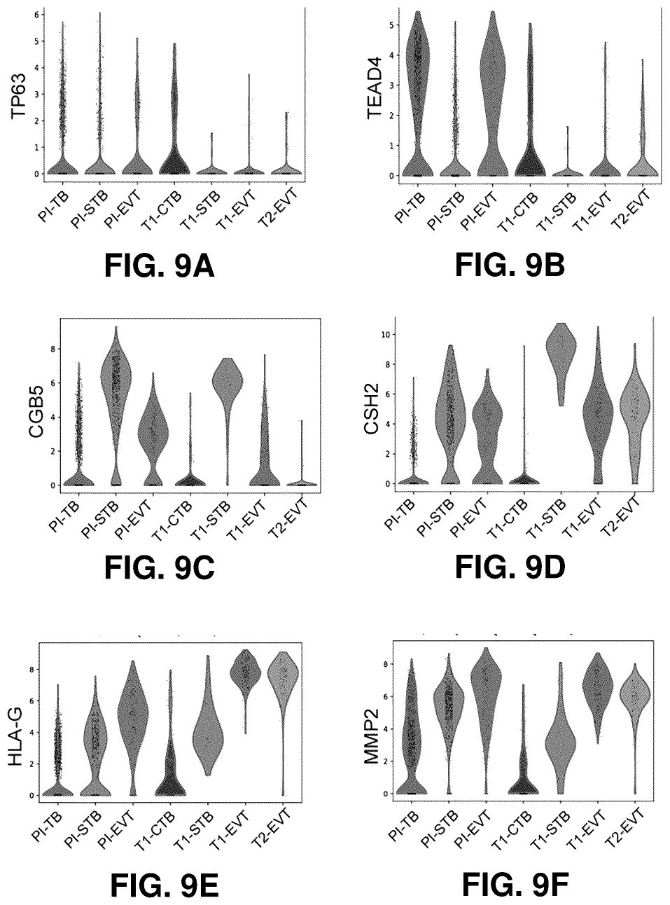

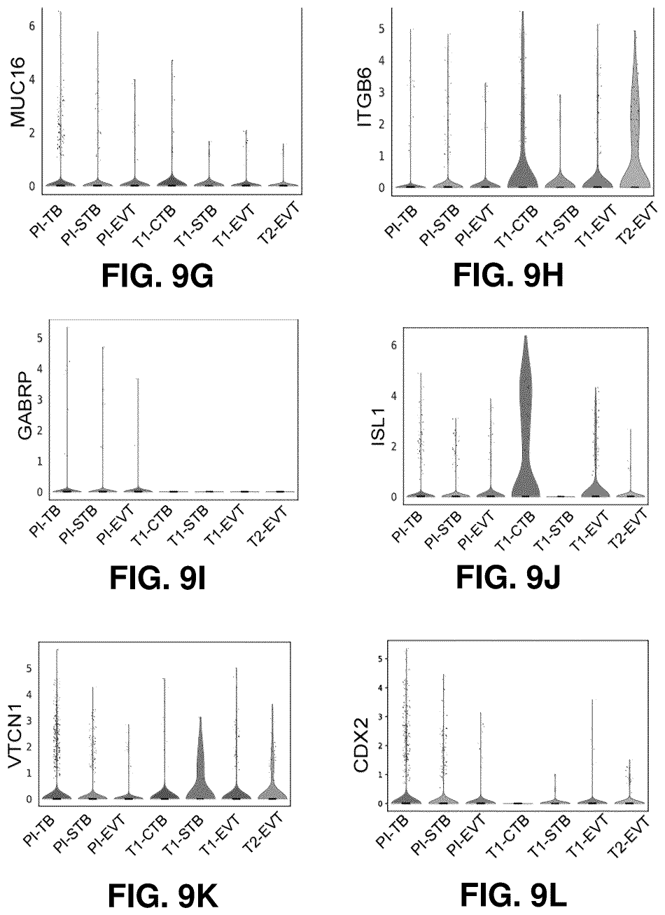

The EPSC-derived trophoblasts were computationally compared to trophoblasts in human peri-implantation embryos and in the placenta in order to validate the lineage identity of the in vitro generated trophoblasts. Extracted were the scRNA-seq data of 4041 peri-implantation extraembryonic cells (embryonic days 6 to 14) in the prolonged culture of human embryos in vitro (Zhou, F. et al. (2019) Nature 572(7771), pages 660-664), and 952 placental cells from first and second trimester pregnancies (Liu, Y. et al. (2018) Cell Research 28(8), pages 819-832). These scRNA-seq datasets were subsequently combined to compute the joint uniform manifold approximation and projection (UMAP). The developmental times were highlighted (in embryonic day or ED, or gestational week W) (FIG. 8A) and the developmental stages and subtypes were annotated (FIG. 8B). The peri-implantation sector contained the trophoblast cells (PI-TB) which possessed sternness and the potency to differentiate into EVT (PI-EVT) and STB (PI-STB) as shown in the same sector (FIG. 8B). Correspondingly, the placenta cells could be identified as either first trimester cytotrophoblast cells (T1-CTB), T1-EVT and T1-STB, or second-trimester placenta EVT (T2-EVT) (FIG. 8B). The trophoblast marker genes were examined in each stage-specific subtypes, which validated their trophoblast identities (FIGS. 9A-9F). Notably, these human in vivo trophoblasts did not appear to highly express the reported putative amnion marker genes MUC16, GABRP or CDX2, whereas some in vivo trophoblasts did express VTCN1, ITGB6 and ISL1 (FIGS. 9G-9L).

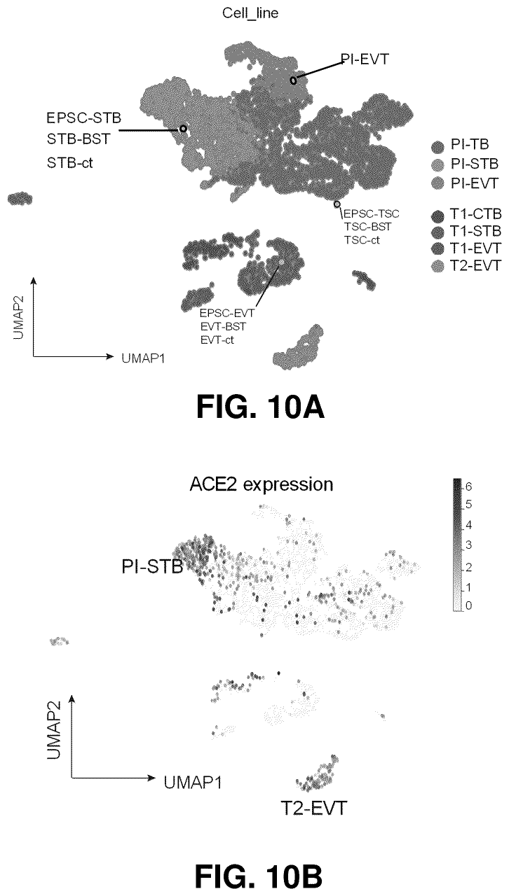

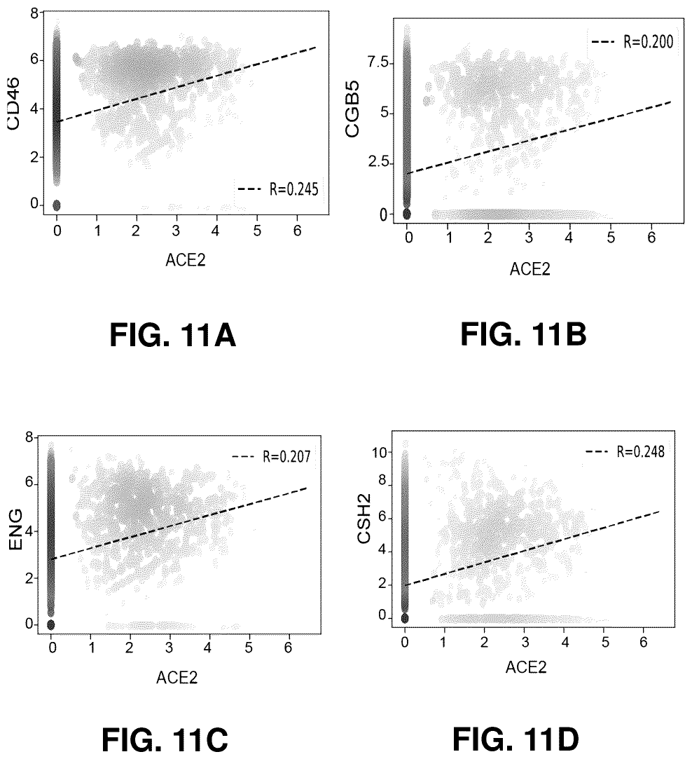

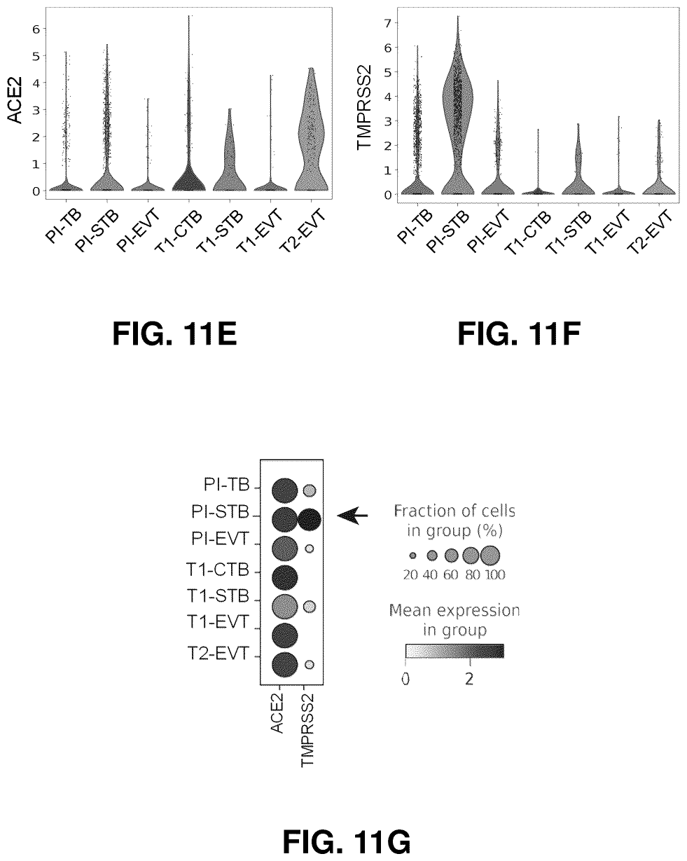

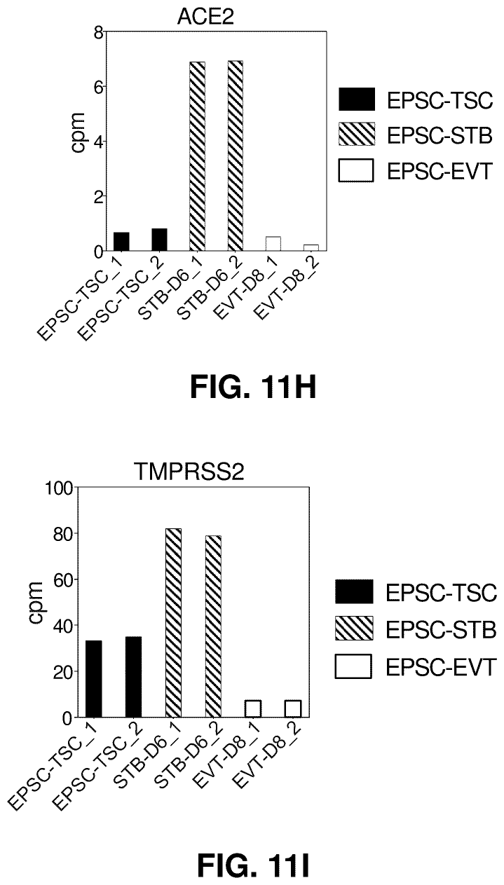

The EPSC-TSCs generated in this study and their STBs and EVTs derivatives, as well as the blastocyst- or cytotrophoblast-derived TSCs (BST-TSC/CT-TSC) and their derived STBs/EVTs as reported in previous studies (Okae, H. et al. (2018) Cell Stem Cell 22(1), pages 50-63. E6), were projected and mapped against the in vivo trophoblast clusters categorized by stage and lineage as described in FIGS. 8A and 8B. All hTSCs (EPSC-TSC, BST-TSC, CT-TSC) were projected in a proximity to PI-TB which possess stemness (Castel, G. et al. (2020). Cell Reports Vol. 33(8), Article 108419), whereas STB cells were projected to PI-STB and EVT cells were projected to T1-EVT, respectively, regardless of cell line origins (FIG. 10A). The resemblance between in vitro and the in vivo counterparts was supported by whole-transcriptome Pearson correlation. Notably, EPSC-TSCs and the STBs and EVTs did not express these putative AME genes at high levels, similar to BST-TSCs, CT-TSCs and their STBs/EVTs (FIGS. 11A-11D).

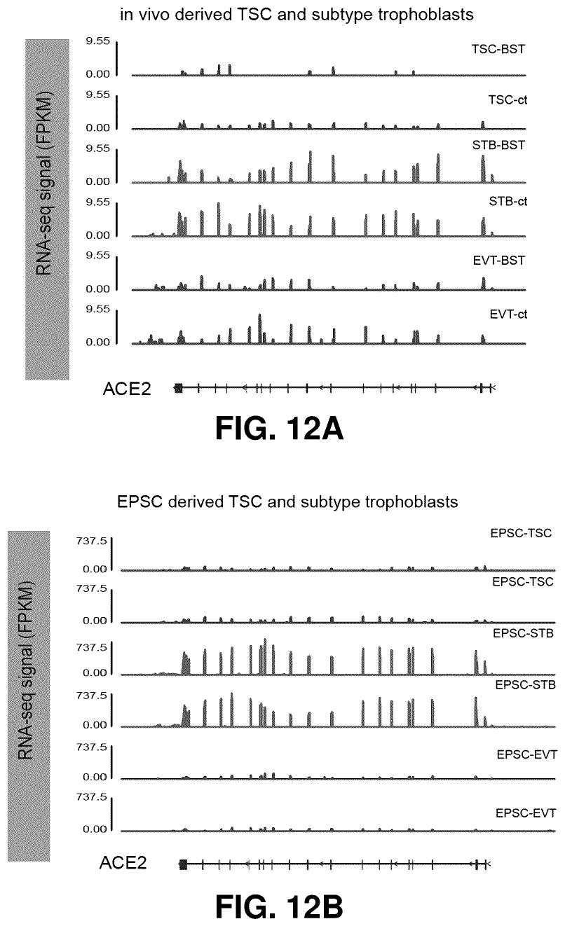

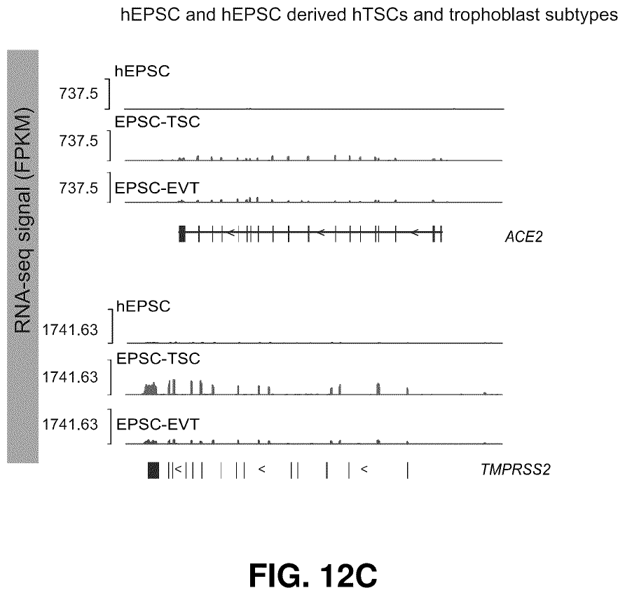

SARS-CoV-2 host factor genes in the in vivo trophoblasts was examined to explore the potential trophoblast susceptibility to SARS-CoV-2. The expression of the recognized SARS-CoV-2 receptor gene ACE2 expression was high in many cells in the PI-STB cluster (FIG. 10B). In T2-EVT cells, ACE2 expression is increased, whereas in PI-STB cells, both ACE2 and TMPRSS2 expression are increased. Correlation analysis between ACE2 and trophoblast subtype markers only detected significant positive correlations with STB markers such as CD46, CGB5, ENG, and CSH2 (r=0.245, 0.200, 0.207, 0.248, respectively, P<0.0001) (FIGS. 11A-11D) but not with EVT genes (Table 4). In the in vivo trophoblast datasets, the PI-STB cluster expressed highest levels of both ACE2 and TMPRSS2, whereas PI-TB, T1-STB and T2-EVT also had some co-expression (FIGS. 11E-11G) similar to previously reported studies (Ashary, N. et al. (2020). Frontiers in Cell and Developmental Biology Vol. 8, Article 783; Chen, W. et al. (2020) Engineering (Beijing) Vol. 6(10), pages 1162-1169). In contrast to ACE2 and TMPRSS2, other reported SARS-CoV-2 receptor-related genes such as BSG (as reported in Wang, K. et al. (2020) Signal Transduction and Targeted Therapy Vol. 5(1), Article 283) and AXL (as reported in Wang, S. et al. (2021) Cell Research Vol. 31, pages 126-140), did not appear to be specifically expressed in any particular cell cluster (FIG. 12A).

In the in vitro cultured trophoblasts, EPSC-TSCs, BST-TSC, CT-TSC, and the STBs and EVTs derived from them, all expressed ACE2 and TMPRSS2 with STBs having the highest levels (FIGS. 11H-11I, 12B, and 12C). The host factor gene expression profiles in both the in vivo and in vitro human trophoblasts highlighted STB's potential susceptibility to SARS-CoV-2 infection.

TABLE 4
Correlation index between ACE2 and Trophoblast markers.

| Marker | coefficient | p_value |
|---|---|---|
| CDX2 | 0.04266543 | 0.00256635 |
| CD46 | 0.24451753 | 7.03E−69 |
| TFRC | −0.0493468 | 0.00048636 |
| GCM1 | −0.0636706 | 6.72E−06 |
| CGB3 | 0.10253989 | 3.79E−13 |
| CGB5 | 0.19950932 | 5.37E−46 |
| SDC1 | 0.10222731 | 4.46E−13 |
| GATA2 | −0.0356747 | 0.01170267 |
| GATA3 | −0.0968373 | 7.03E−12 |
| TP63 | −0.0269452 | 0.05692952 |
| TEAD4 | −0.1328758 | 4.15E−21 |
| ACE2 | 1 | 0 |
| ENG | 0.20688926 | 2.11E−49 |
| ITGA5 | 0.10266201 | 3.55E−13 |
| ITGA6 | −0.0676585 | 1.71E−06 |
| CSH1 | 0.18957838 | 1.27E−41 |
| CSH2 | 0.24808141 | 6.54E−71 |
| HLA-G | 0.0706351 | 5.85E−07 |
| MMP2 | 0.03941273 | 0.00534726 |

### Example 3: hTSCs are Susceptible to SARS-CoV-2 Infection

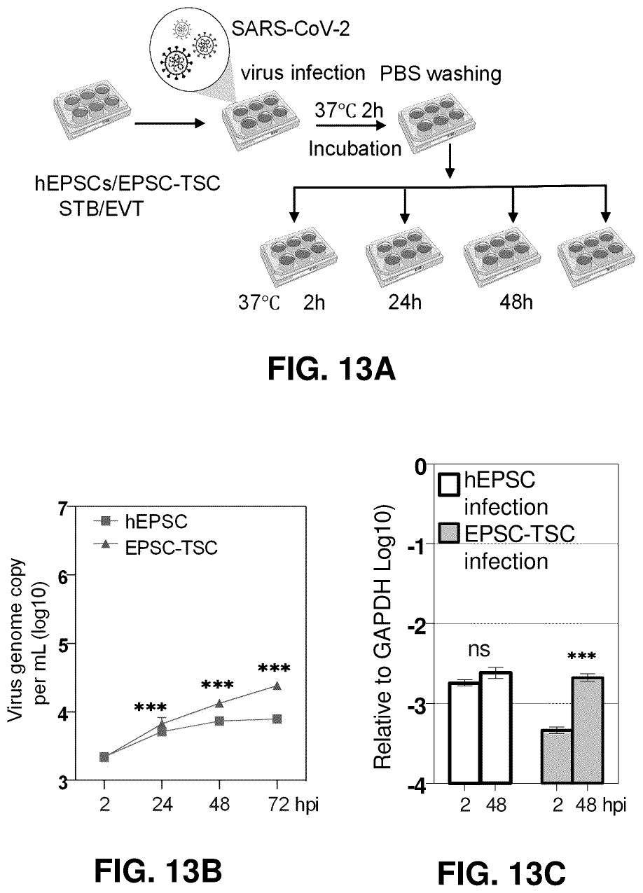

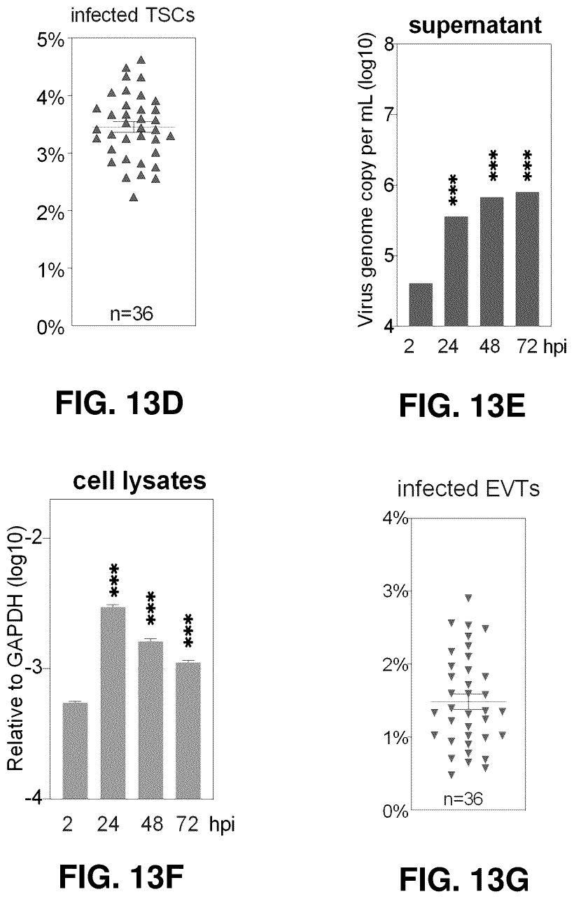

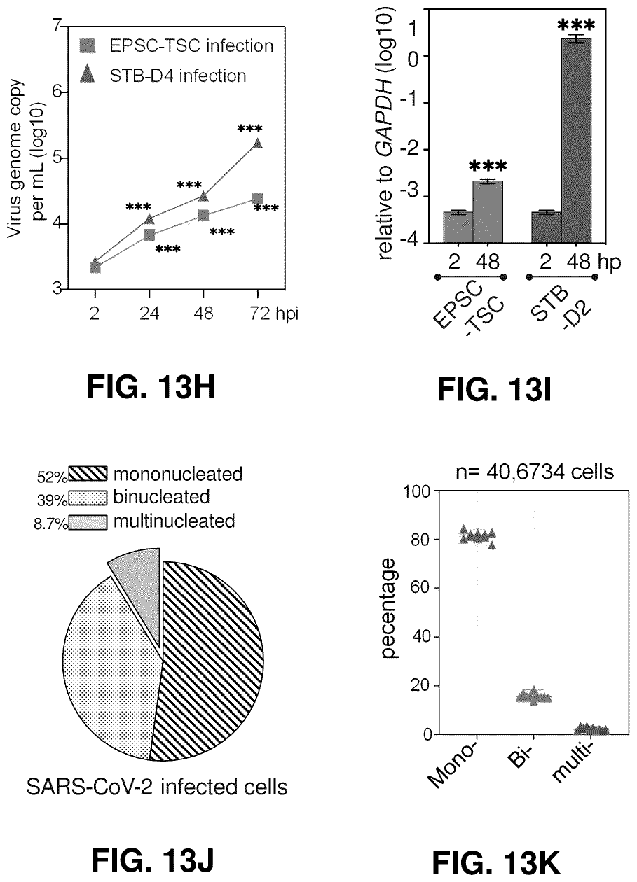

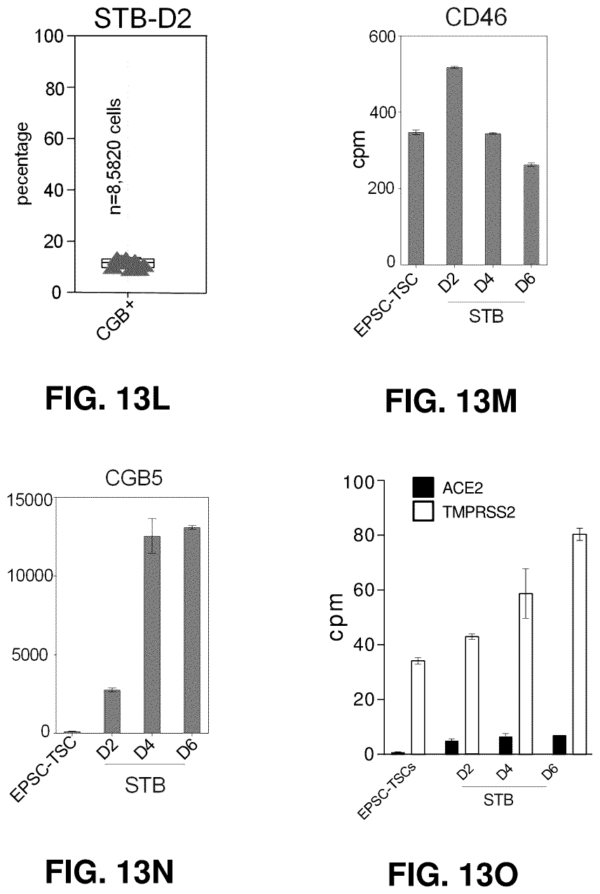

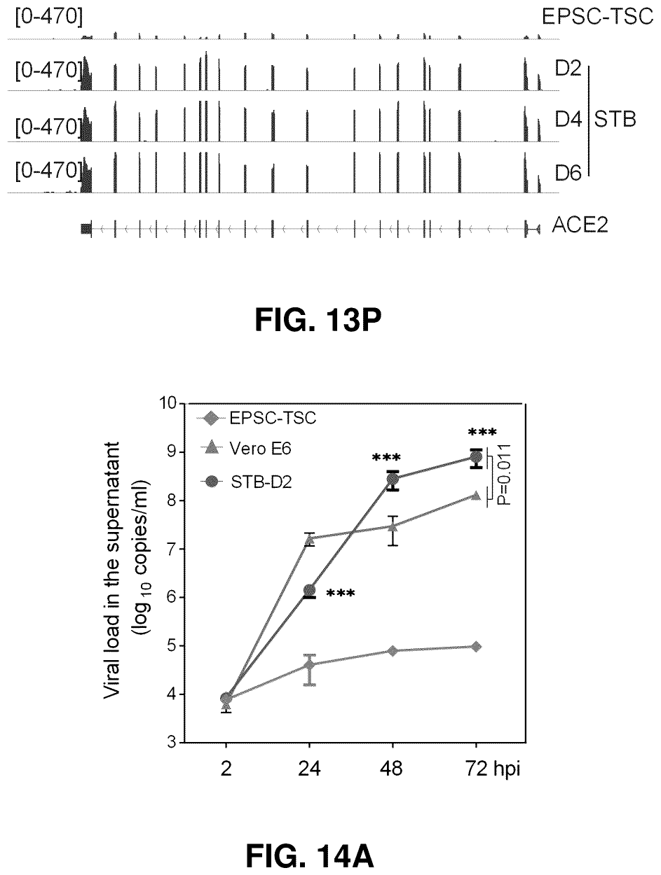

To experimentally investigate trophoblast susceptibility to SARS-CoV-2 infection, the viral replication kinetics among hEPSCs, EPSC-TSCs and the derived STBs and EVTs were compared following infection with the SARS-CoV-2 virus. Briefly, cells were infected with SARS-CoV-2 (SARS-CoV-2 HKU-001 a strain; GenBank accession number MT230940) for 2 hours followed by incubation in fresh medium for another 24, 48, or 72 hours or hpi (hours post infection) (FIG. 13A). The supernatant and cell lysates were collected for viral genome and antigen detection. hEPSCs did not express ACE2 (FIG. 12C) thus were poorly infected by SARS-CoV-2, evidenced by the marginally increased viral genome in the supernatant or cell lysate (FIGS. 13B and 13C), neither was the viral N protein was visible by immunofluorescence staining. In line with relatively low ACE2 and TMPRSS2 expression levels, SARS-CoV-2 infected EPSC-TSCs, but with only about 3-4% cells being positive for the viral N protein antigen (FIG. 13D). Only a small number of hTSCs express ACE2 and can therefore be infected by the virus (data not shown). The infected EPSC-TSCs were positively stained for ACE2 and the pan-trophoblast marker KRT7. Intriguingly, the infected EPSC-TSCs expressed the blastocyst trophectoderm (TE) marker ENPEP (as reported in Jo, S. et al. (2021). Cell Stem Cell Vol. 28(6), pages 1023-1039.e1013), which is a candidate co-receptor for SARS-CoV-2 (Qi, F., et al. (2020). Biochemical and Biophysical Research Communication Vol. 526(1), pages 135-140. Similar to EPSC-TSCs, about 0.5-1% of BST-TSCs derived from the human blastocyst (Okae, H. et al. (2018) Cell Stem Cell 22(1), pages 50-63.e6) were infected by SARS-CoV-2 (FIG. 13E) and were stained positive for KRT7, ACE2 and ENPEP. The BST-TSC cultured in the present study appeared to contain some differentiated trophoblasts, which may account for lower percentages of ACE2+ cells.

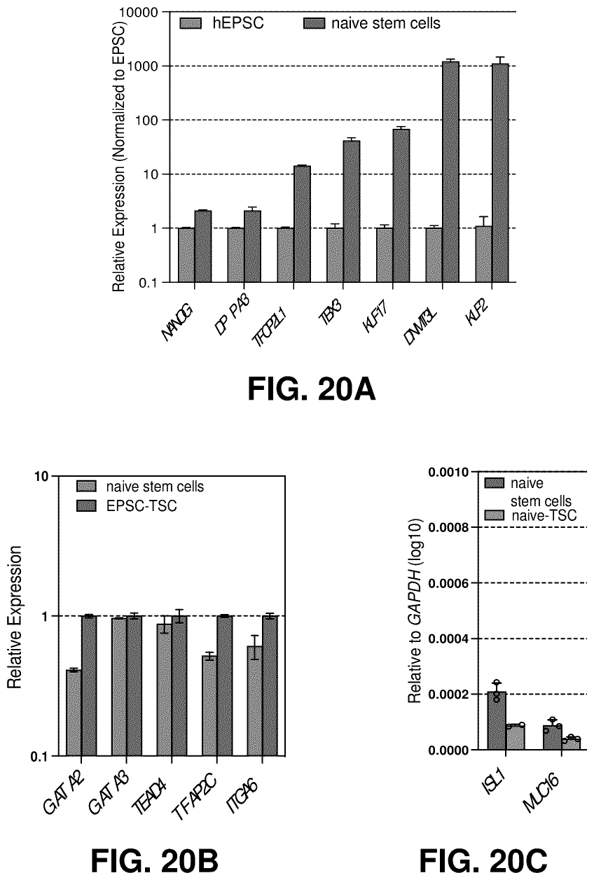

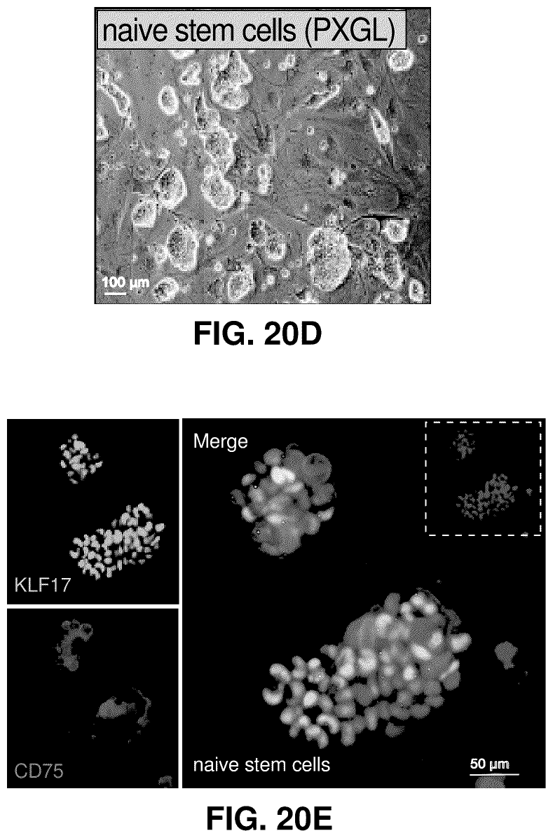

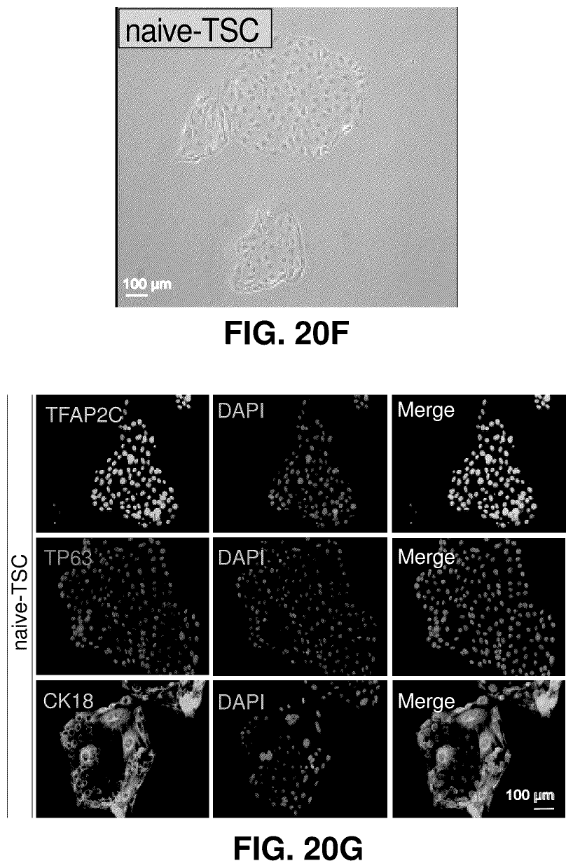

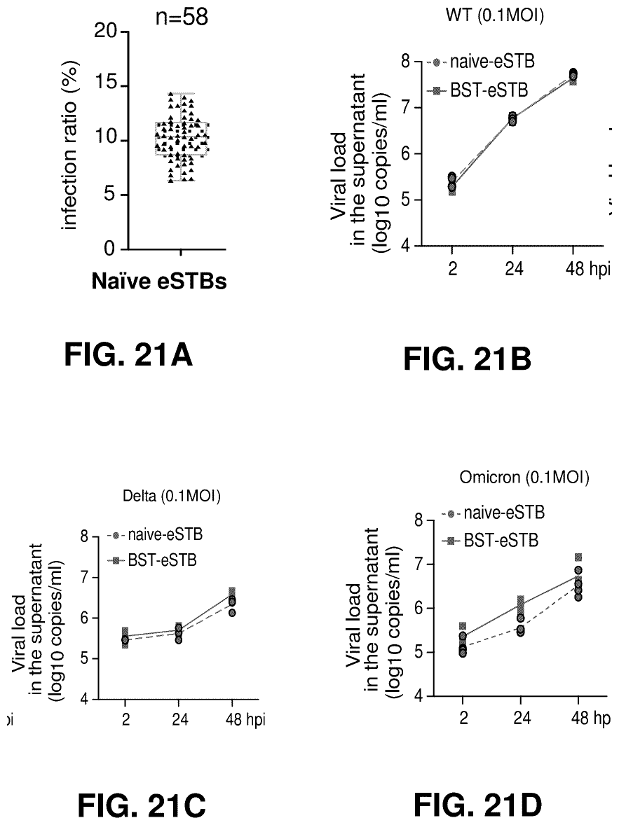

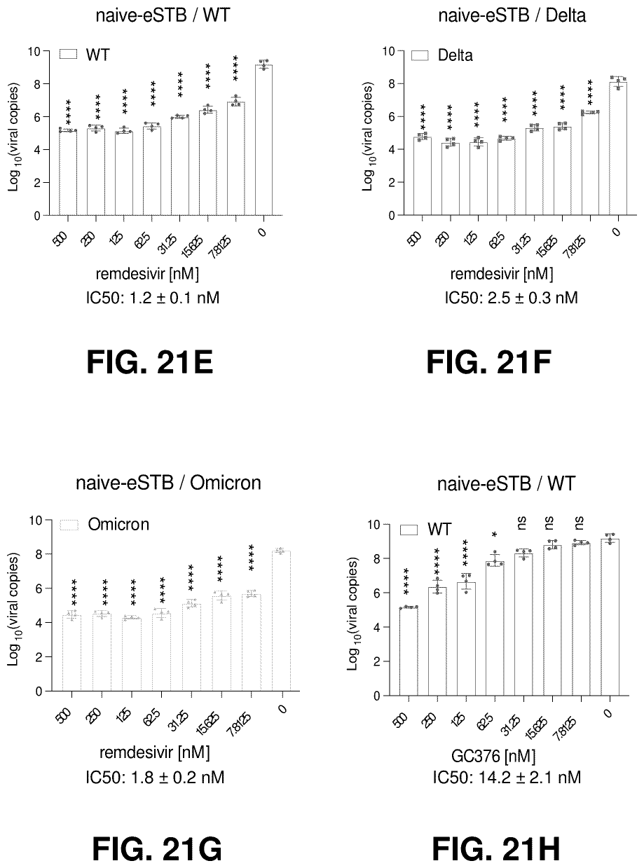

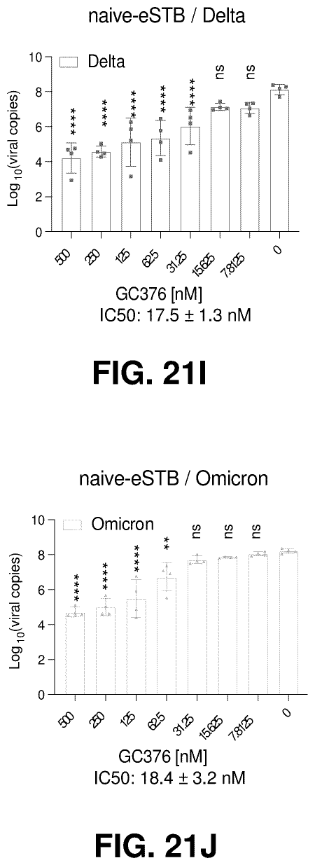

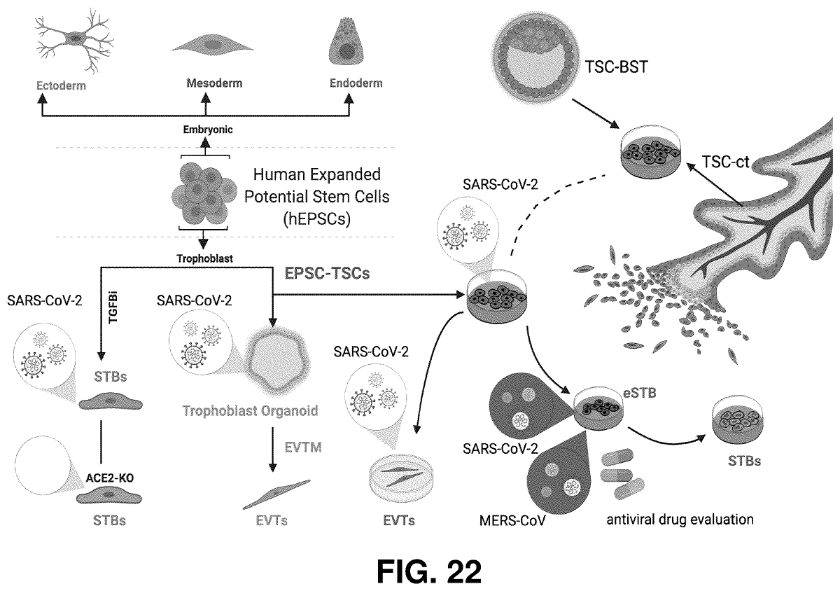

To experimentally investigate hTSCs derived from naïve stem cells in SARS-CoV-2 infection, human primed ESCs (H1) were converted to naïve stem cells in the PXGL culture condition (Guo, G. et al. (2021). Cell Stem Cell 28(6), pages 1040-1056. E6) (FIG. 20D). These cells expressed the naïve pluripotency genes DPPA3, TFCP2L1, TBX3, KLF17, KLF2, DNMT3L and the surface marker CD75 (FIGS. 20A and 20E), and were used to derive naïve-TSCs which formed typical cobblestone-shaped TSC colonies (FIG. 20F), were morphologically reminiscent of EPSC-TSCs and other hTSCs (FIG. 20F), and expressed the trophoblast genes GATA2, GATA3, TFAP2C, TP63, TEAD4, CK18 (FIGS. 20B and 20G) at high levels but low or undetectable putative AME genes ISL1 or MUC16 (FIG. 20C). Again, 2-3% of the naïve-TSCs were infected by SARS-CoV-2 (FIG. 21A) and expressed ACE2. The infected cells also co-expressed the TE marker ENPEP.

The presence of a small number of ACE2-positive and SARS-CoV-2 susceptible cells in hTSCs revealed the heterogeneity of the current hTSC cultures, which warrants future investigation. The observation that hTSCs are similar to PI-TB at the transcriptome level (Castel, G. et al. (2020). Cell Reports Vol. 33(8), Article 108419) (FIG. 10A) and that ACE2-positive hTSCs were susceptible to SARS-CoV-2 indicate that human peri-implantation embryos potentially are at risk of SARS-CoV-2 infection.

### Example 4: eSTBs are Highly Susceptible to SARS-CoV-2 Infection

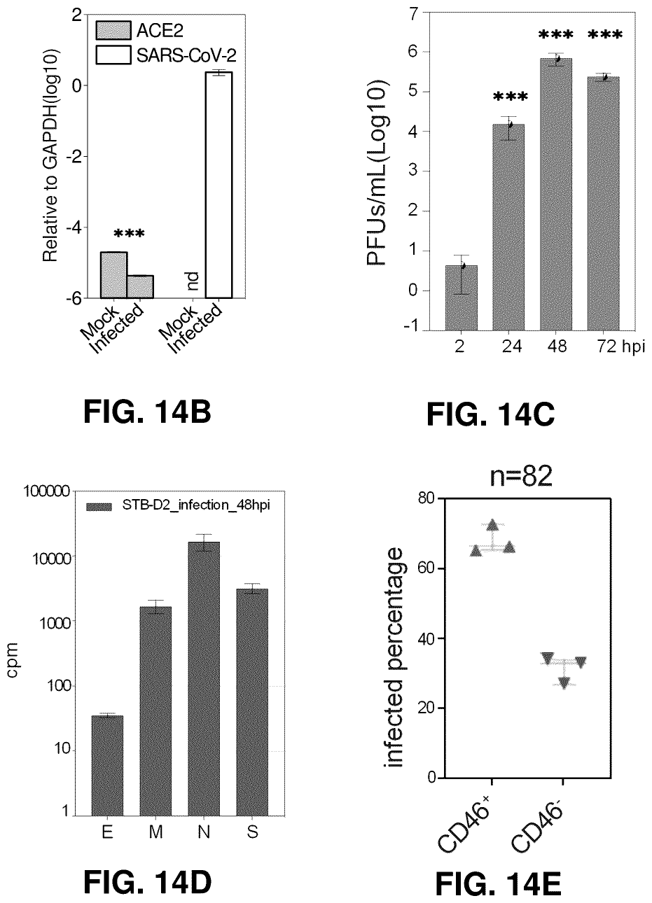

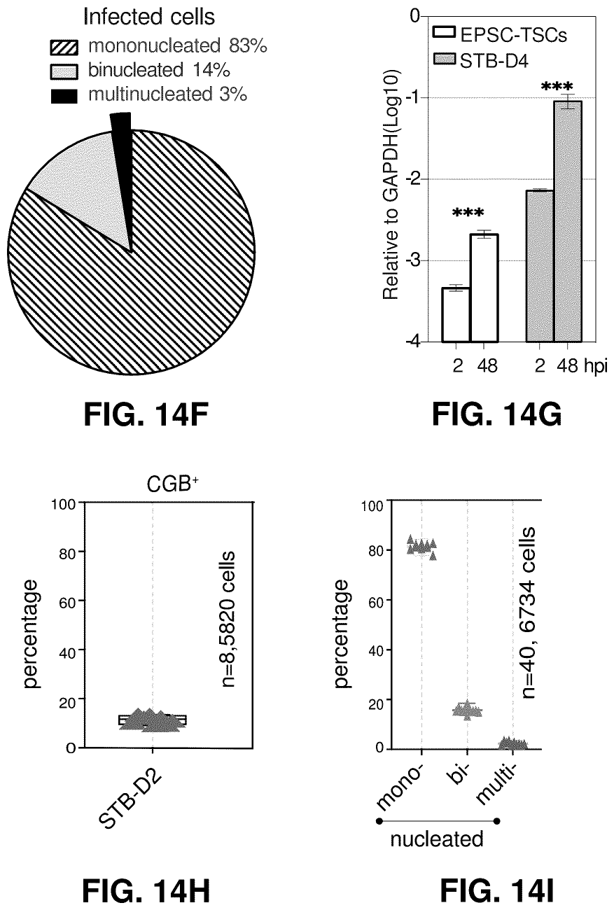

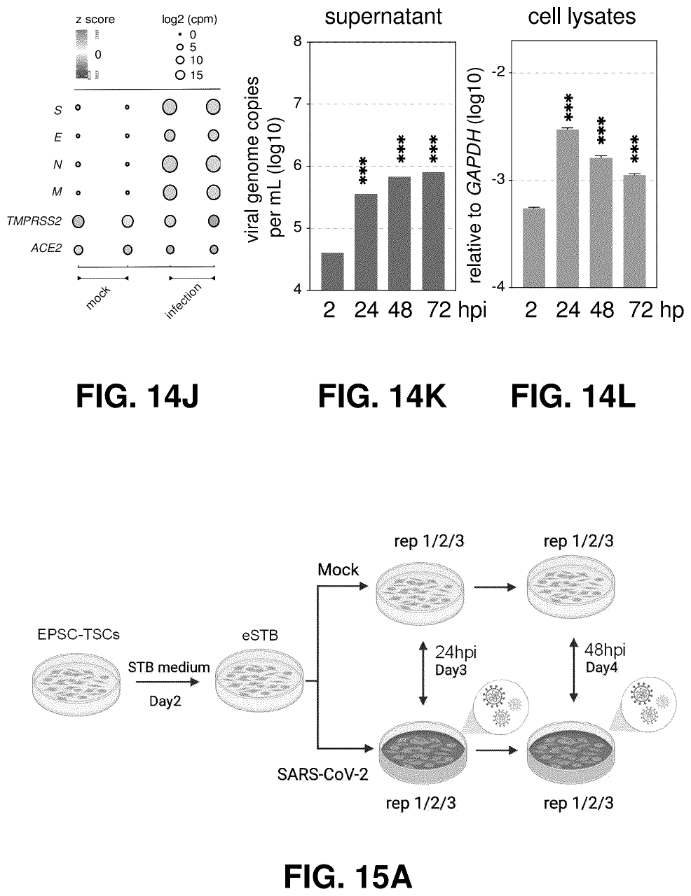

The in vivo STBs in the peri-implantation embryos and those generated from hTSCs co-expressed ACE2 and TMPRSS2 (FIGS. 11E-11G; and 11H-11I). In hTSC differentiation toward STBs, expression levels of both ACE2 and TMPRSS2 were substantially increased starting from day 2 (STB-D2) (FIGS. 14A and 14B), in line with more efficient infection by SARS-CoV-2 in STBs than hTSCs (FIGS. 13H and 13I). It was observed that the virus-infected cells were generally positive for early STB markers (SSEA4 and CD46) (Gamage, T. K., et al. (2016) Hum Reprod Update Vol. 23, pages 77-103; Holmes, C. H., et al. (1992) European Journal of Immunology Vol. 22, pages 1579-1585) (FIG. 14E). Indeed, multinucleated and CGB⁺ mature STBs did not express high levels of ACE2 and only accounted for minor population of the infected cells (FIG. 13J). These results indicate that SARS-CoV-2 appears to preferentially infect immature or early STBs than multinucleated mature STBs.

In STB-D2, most cells were mononucleated and negative for CGB and only 3% of cells were multinucleated (FIGS. 13K and 13L). The early STB gene CD46 was transiently up-regulated in STB-D2 whereas the mature STB gene CGB markedly increased after D2 (FIGS. 13M and 13N). Notably, ACE2 expression was rapidly upregulated in STB-D2, together with TMPRSS2 (FIGS. 13O and 13P). Thus, STB-D2 cells were used for the subsequent infection experiments and are empirically referred to as early STBs (eSTBs).

Following eSTB cell infection with SARS-CoV-2 (MOI: 0.1), supernatants were collected at 2 h.p.i., 24 h.p.i., 48 h.p.i., and 72 h h.p.i. and quantified for viral RNA loads (FIG. 14A). The results revealed that eSTBs produced high amounts of supernatant viral RNA at 48- and 72-h.p.i (FIG. 14C). Quantification of immunofluorescence staining at 24 h.p.i. showed substantially higher rates of infection than that of EPSC-TSCs (FIG. 14F). In line with this observation, a >3 log₁₀ viral genome copies in the cell lysates were documented when comparing eSTBs to and hTSCs (FIG. 14E). A continuous release of infectious virus particles from the infected eSTBs was also detectable in plaque assays (FIG. 14G). The robust production of SARS-CoV-2 was further revealed in RNAseq where abundant transcripts of viral Envelope (E), Membrane glycoprotein (M), Nucleocapsid (N) and Spike (S) genes were detected, whereas ACE2 and TMPRSS2 were decreased (FIG. 14D) (Banu, N. et al. (2020). Life Sciences Vol. 256, Article 117905). Quantification of immunostained SARS-CoV-2 N protein revealed that the virus infected cells were primarily CD46⁺ whereas those multinucleated cells were rarely infected (FIG. 14E).

To exclude the possibility that eSTB's susceptibility to SARS-CoV-2 is cell line specific, eSTBs were generated from naïve-TSCs and substantial infection was observed (FIG. 14H). Quantification of immunostained infected cells demonstrated that they were mostly mononucleated and CGB− cells.

Besides STBs, hTSCs could efficiently generate EVTs. EPSC-EVTs expressed low levels of ACE2 and TMPRSS2 (FIG. 14J). Consistent with low ACE2 expression, only 1%-2% of EVTs were infected by SARS-CoV-2 but virus gene expression was still detectable (FIGS. 14K and 14L).

### Example 5: Susceptibility of Human Trophoblasts to SARS-CoV-2 Variants of Concern

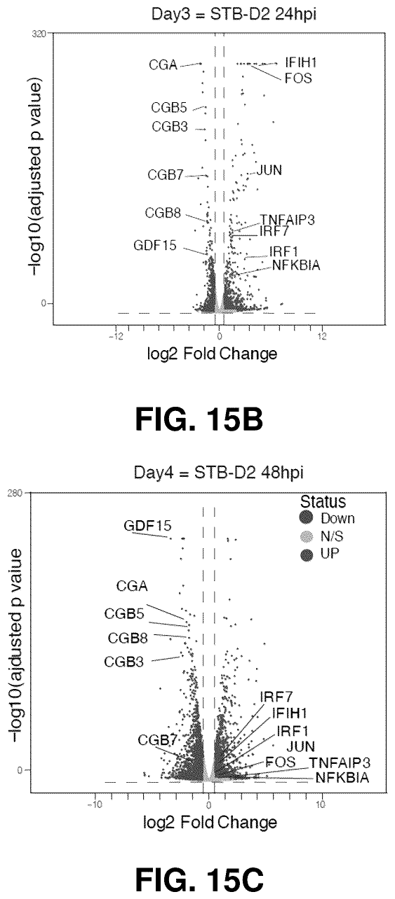

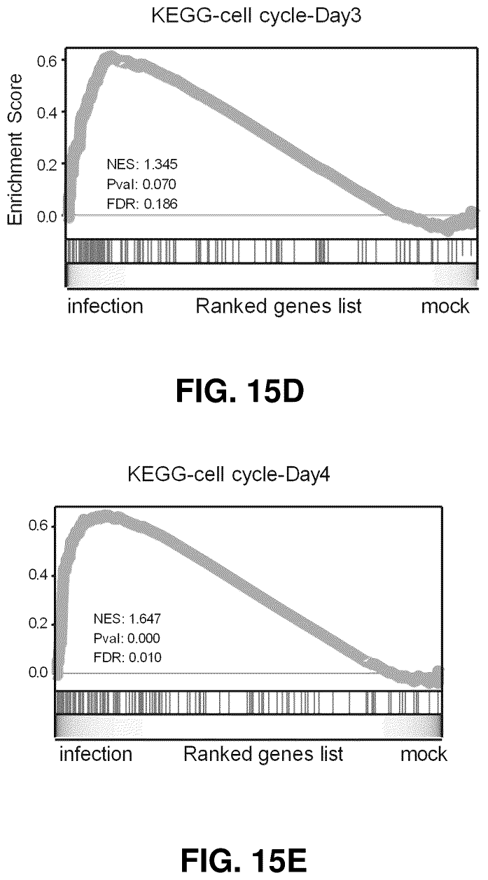

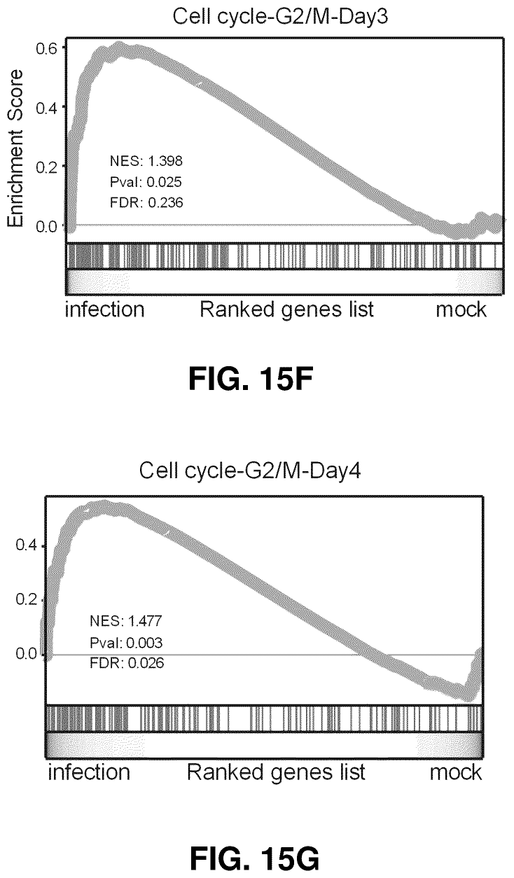

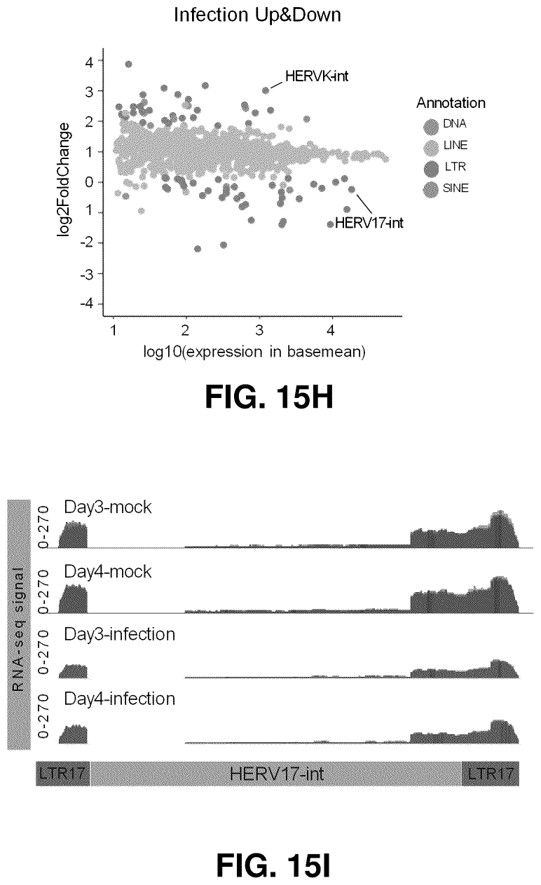

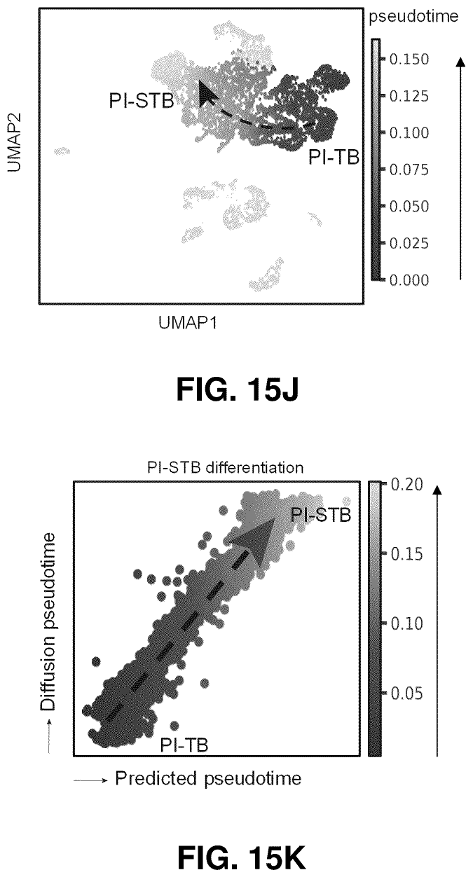

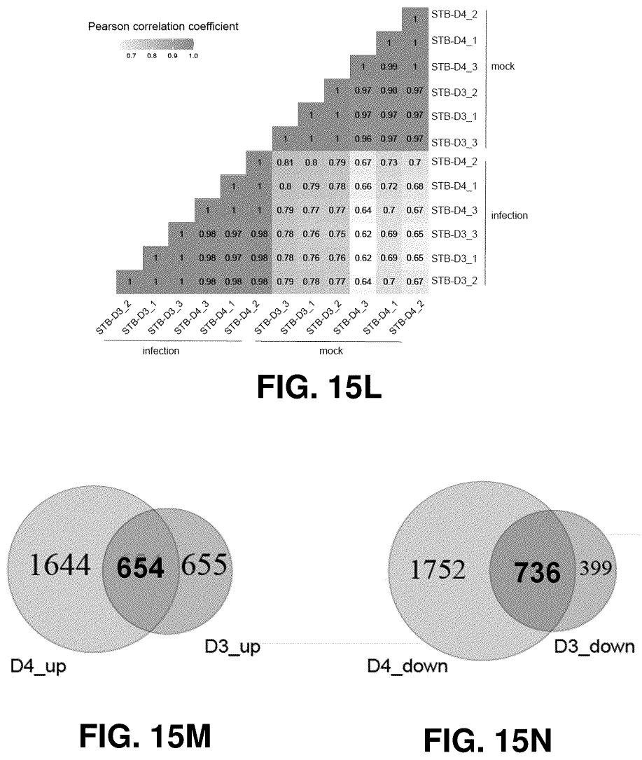

Since its emergence in 2019, SARS-CoV-2 variants have arisen with mutations throughout its genome that impact virus replication, infectivity, transmission, and infection- and vaccine-induced immunity (DeGrace, M. M. et al. (2022) Nature doi: 10.1038/s41586-022-04690-5). The Delta variant was the previous predominant circulating SARS-CoV-2 strain, which was overtaken by the Omicron variants since late November 2021. Utilizing area under the curve (AUC) quantification of viral RNA genome copies generated over a period of 48 hours, it was observed that both the Delta (B.1.617.2) and Omicron (B.1.1.529) variants replicated robustly in EPSC-eSTB cells compared to Vero E6 cells (FIGS. 15A and 15B; Shuai, H. et al. (2022) Vol. 603(7902): pages 693-699).

Among the three lineages of SARS-COV-2, less viral NP antigen in EPSC-eSTB cells were found in Delta- (˜18.8%) and Omicron-infected cells (˜22%) when compared to that of the SARS-CoV-2 wild type (WT) (˜54%) (FIGS. 15C-15E), which was confirmed by quantification of the viral genome (FIG. 15F). In line with a previous in vivo study (Banu, N. et al. (2020). Life Sciences Vol. 256, Article 117905), ACE2 expression was significantly downregulated in EPSC-eSTB following either Delta or Omicron infection (FIG. 15G). Similar phenotypes were observed in eSTBs generated from additional EPSC-TSC lines (EPSC-TSC 3 #, and hiEPSC-TSC (C5) (Gao, X. et al. (2019) Nature Cell Biology 21(6), pages 687-699)) and from naïve-TSCs and BST-TSCs for their susceptibility to the WT, Delta, and Omicron variants. Thus, eSTB's susceptibility to SARS-CoV-2 infection is independent of cell origin and genetic background.

### Example 6: SARS-CoV-2 Infection Impairs hEPSC-TSC Differentiation to STBs and Induces Potent Innate Immune Response

The global gene transcriptional changes in infected EPSC-eSTBs were explored (FIG. 15A). Pearson correlation and cluster analysis revealed the SARS-CoV-2 infected cells (24 h.p.i., or STB-D3; 48 h.p.i. or STB-D4) were clustered together and separated from the mock infection controls (FIG. 15L). Comparative transcriptomic analysis identified 654 upregulated differentially expressed genes (DEGs) and 736 downregulated DEGs in SARS-CoV-2 infected cells (FIGS. 15M and 15N).

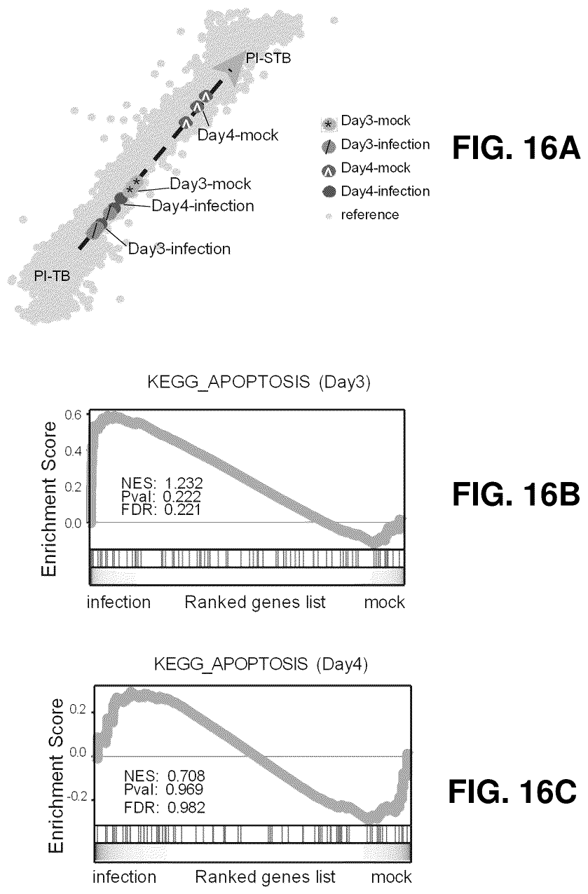

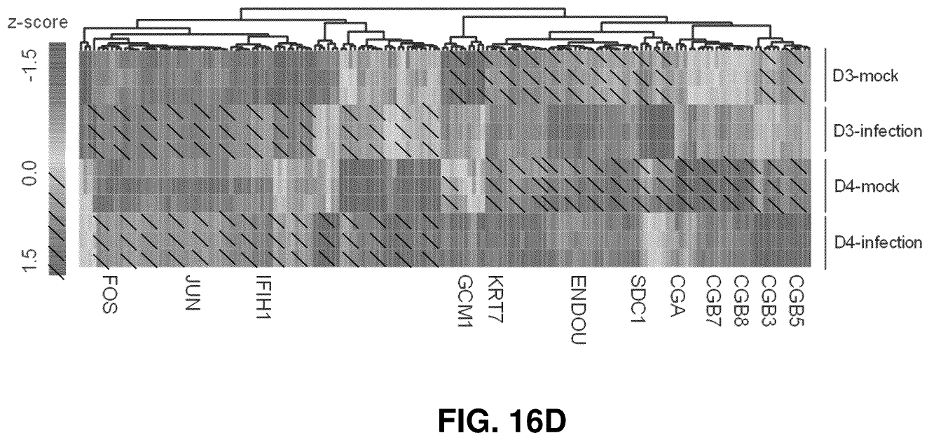

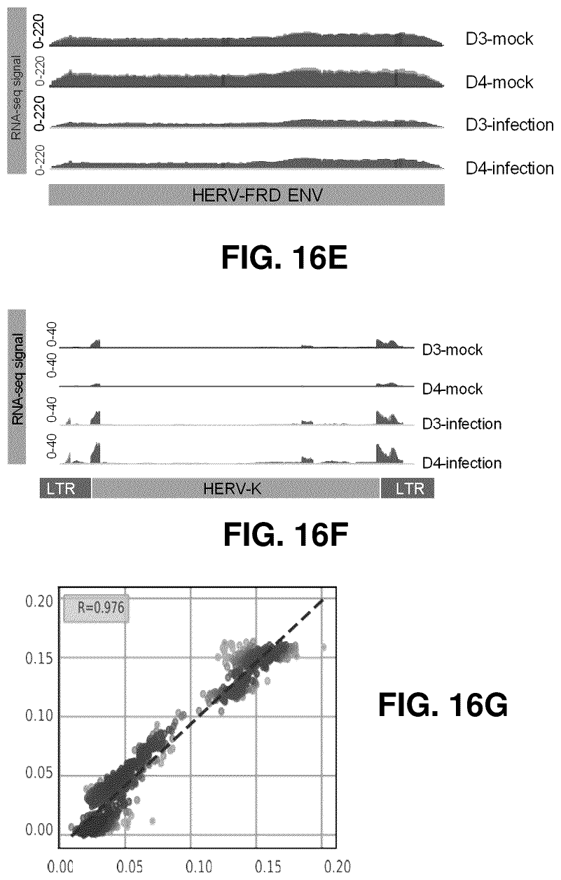

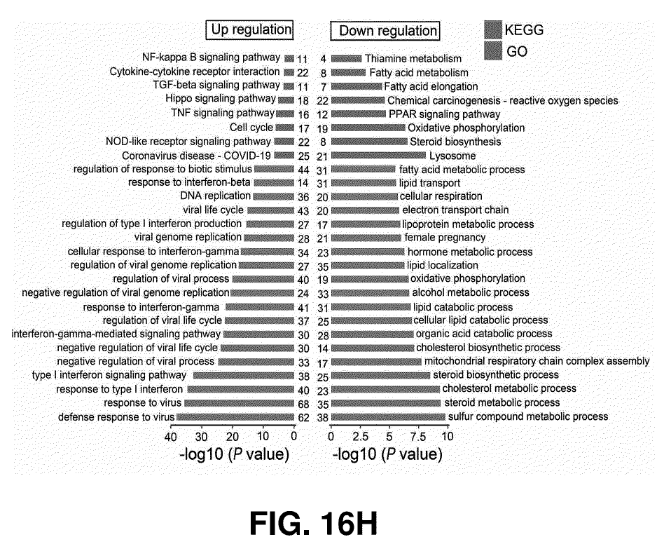

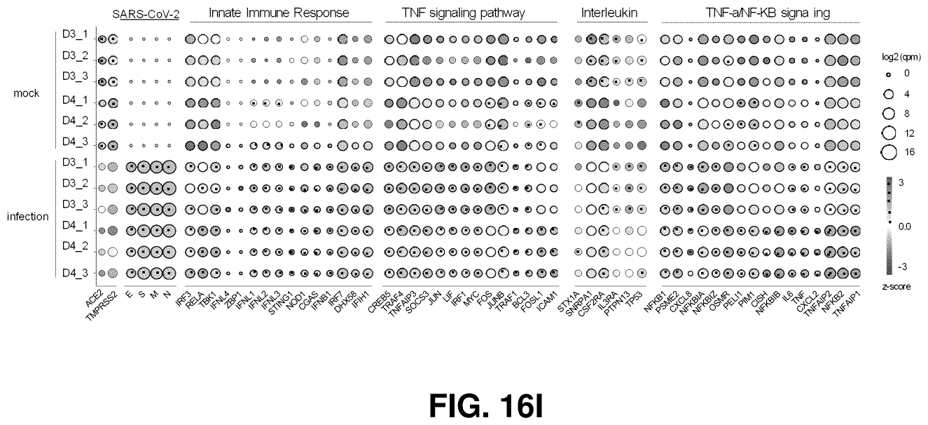

Among the genes that were significantly decreased in the SARS-CoV-2 infected cells were CGA, CGBs (CGB3, 5, 7, 8), GCM1, SDC1 and ENDOU and others that are highly expressed in mature and multinucleated STBs (FIGS. 15B, 15C, and 16D), indicating that the infection caused a possible developmental blockage. Gene Set Enrichment Analysis (GSEA) revealed a significant enrichment of cell cycle in particular G2-M related genes in the infected cells compared to the mock control (FIG. 16D). The lack of mature STB signature and higher cell cycle genes in infected cells are consistent since mature and multinucleated STBs are known to have minimal cell cycle activities.

Interestingly, GSEA revealed that SARS-CoV-2 infection did not substantially affect the infected cell viability as apoptosis-related genes were not significantly changed in the infected cells (FIG. 16B). In contrast, the infected Vero E6 cells had enriched expression of apoptosis-related genes (Riva, L. et al. (2020). Nature Vol. 586(7827): pages 113-119) (FIG. 16C). Experimentally, Vero E6 cells mostly detached 72 h.p.i. due to cell death whereas eSTB did not show obvious cytopathogenic effect (CPE) (FIG. 16G). Furthermore, TUNEL cell apoptosis assay confirmed that distinct from Vero E6 cells, eSTB did not undergo appreciable apoptosis 72 h.p.i. (FIG. 16G). Therefore, the lack of mature STBs in the infected cells is more likely caused by developmental block.

The fusion of uninfected eSTBs to form multinucleate STBs requires endogenous retrovirus (HERV) proteins Syncytin-1, an envelope gene of HERV-W, and Syncytin-2 produced from HERV-FRD (Dupressoir et al. (2005) Proceedings of the National Academy of Sciences Vol. 102(3): pages 725-730). SARS-CoV-2 infection of eSTBs resulted in lower levels of both HERV-W (HERV17-int, Syncytin-1) and HERV-FRD (Syncytin-2) (FIG. 16E) but substantially increased HERV-K expression (FIGS. 15H, 15I, 16E, and 16F). HERV-K is known to be exclusively expressed in cytotrophoblast cells (progenitors) in the human placenta (Kämmerer et al., (2011) Journal of Reproductive Immunology Vol. 91(1-2), pages 1-8). These data indicate that SARS-CoV-2 infection impaired eSTB differentiation and maturation.

To further investigate eSTB development following the infection, the transcriptomic pseudo time of the mock and infected STBs were compared. At the transcriptome level, EPSC-TSCs resembled PI-TB, and TSC-derived STB to PI-STB as described above in FIG. 10A. The PI-TB and PI-STB differentiation process was extracted from the in vivo trophoblast scRNA-seq data (Zhou, F. et al. (2019) Nature Vol. 572, pages 660-664) to generate a pseudo time trajectory, which increased from the immature PI-TB to the relatively mature PI-STB (FIG. 15K). Using machine learning methods, the mock and virus-infected STB cells (STB-D3 and STB-D4) were mapped to the PI-TB to PI-STB pseudotime trajectory and discovered that the infected cells were closer to PI-TB along the pseudotime trajectory compared to the mock control cells (FIG. 16A). This result again demonstrates that SARS-CoV-2 infection of eSTBs adversely affected normal trophoblast development and function, which may implicate potential COVID-19 infection risk in early pregnancy, which may be asymptomatic and unnoticed (Zhou, J. et al. (2021) The Journal of Infectious Diseases Vol. 224 (Suppl. 6), pages 5660-5669).

SARS-CoV-2 infection caused a strong innate immunity response. Genes encoding interferon signaling components (IFNL1, IFIH1) and genes associated with TNFα signaling via NF-κB such as TNFAIP3 and NFKBIA were all up regulated in the infected cells (FIGS. 15B, 15C, and 16D). Gene Ontology (GO) term analysis found enriched GO terms related to virus cellular response and immune response pathways in SARS-CoV-2 infected STBs (FIG. 16H). As expected, KEGG pathway analysis revealed that the coronavirus disease-COVID19 pathway was among the over-represented ones (FIG. 16H). Bubble plot analysis further demonstrated in the upregulated genes an enrichment of those associated with innate immune response, interleukin and TNF signaling pathways (FIG. 16I).

The double-stranded RNA (dsRNA), generated during coronavirus genome replication and transcription, could be recognized by melanoma differentiation gene 5 (MDA5/IFIH1) in the cytoplasm to trigger innate immune activation upon coronavirus infection (Kindler, E. et al. (2016) Advances in virus research Vol. 96, pages 219-243; Li, J. et al. (2010) Journal of virology Vol. 84, pages 6472-6482; Yin, X. et al. (2021). Cell Reports Vol. 34(2), Article 108628). IFIH1 was highly up regulated in the infected cells (FIG. 16I). Meanwhile, DNA is not known to be involved in the SARS-CoV-2 life cycle. Consistently, genes encoding cGAS and STING1, both being components of the cGAS-STING pathway of the innate immune system detecting cytosolic DNA, were not substantially altered (FIG. 16I).

In response to viral infections, interferons (IFNs) initiate a signaling cascade that stimulates the expression of many genes and creates an intracellular antiviral defense. Type III IFNs have are important antiviral factors functions (Ye, L., et al. (2019) Nature Reviews Immunology Vol. 19, pages 614-625). Particularly, IFN-λ1 is known to be constitutively released from human placental trophoblasts to protect the fetus from viral infections (Bayer, A., et al. (2016) Cell Host Microbe Vol. 19, pages 705-712; Chen, J., et al. (2017) Cell Rep Vol. 21, pages 1588-1599). Type III IFNs have also been shown to restrict SARS-CoV-2 infection in airway and intestinal epithelia (Stanifer, M. L., et al. (2020) Cell Rep Vol. 32, Article 107863; Vanderheiden, et al. (2020) Journal of Virology Vol. 94). Infected cells expressed high levels of genes encoding Type III IFN including IFN-λ1 (IL-29 or IFNL1), IFN-λ2 (IL-28A or IFNL2), IFN-λ3 (IL-28B or IFNL3) and IFN-λ4 (IFNL4) (FIG. 16I). 2′-5′-oligoadenylate synthetase 1 (OAS1) is a recently identified IFN signaling downstream gene to stimulate Rnase L and to specifically inhibit the virus (Wickenhagen et al., 2021). It was highly expressed in the infected eSTBs (FIG. 16I).

In line with that Vero E6 cells are genetically defective in interferon signaling and TMPRSS2 (Osada, N., et al. (2014) DNA Res Vol. 21, pages 673-683; Sasaki, M., et al. (2021) PloS Pathog Vol. 17, Article e1009233), they expressed ACE2 but not TMPRSS2 (Riva, L., et al. (2020) Nature Vol. 586, pages 113-119). In the infected Vero E6 cells, the interferon genes IFNb1, IRF3, and TBK1 showed no response and the antiviral effector OAS1 only had mild upregulation).

The relatively high Omicron replication in TMPRSS2-deficient Vero E6 cells, is possibly due to Omicron infection being more dependent on cathepsins or other endosomal proteases than the other variants of concern (Beumer et al., 2021; Hui et al., 2022; Koch et al., 2021; Shuai et al., 2022). Vero E6 expressed Cathespin L (CTSL) and Cathepsin L2 (CTSV), which were downregulated following the infection. Similarly, eSTBs expressed both Cathepsin genes and the infected cells had decreased expression (FIG. 16I), which may be a mechanism for Omicron replication in eSTBs.

### Example 7: Remdesivir and GC376 Effectively Eliminate eSTB SARS-CoV-2 and Variant Infection

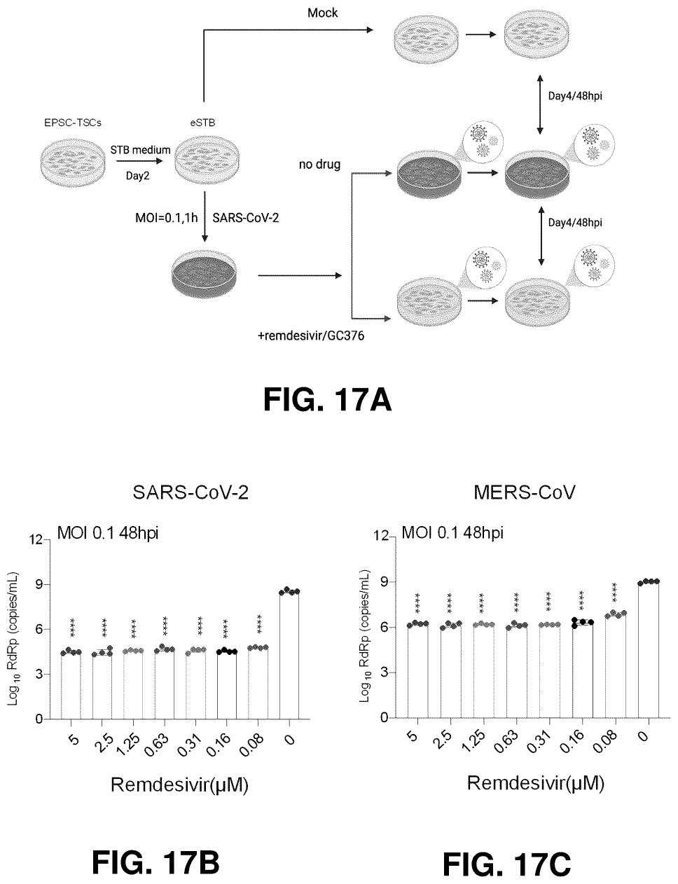

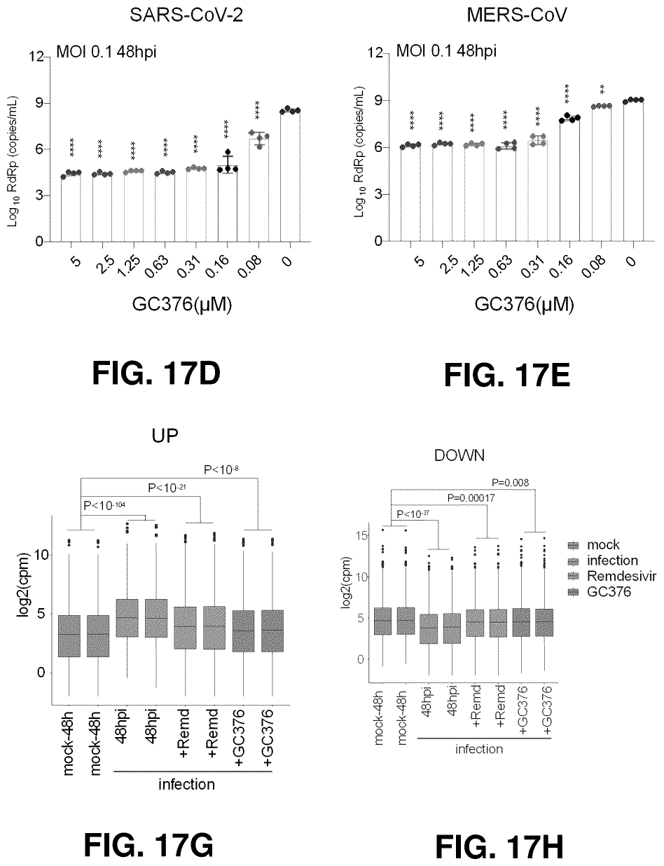

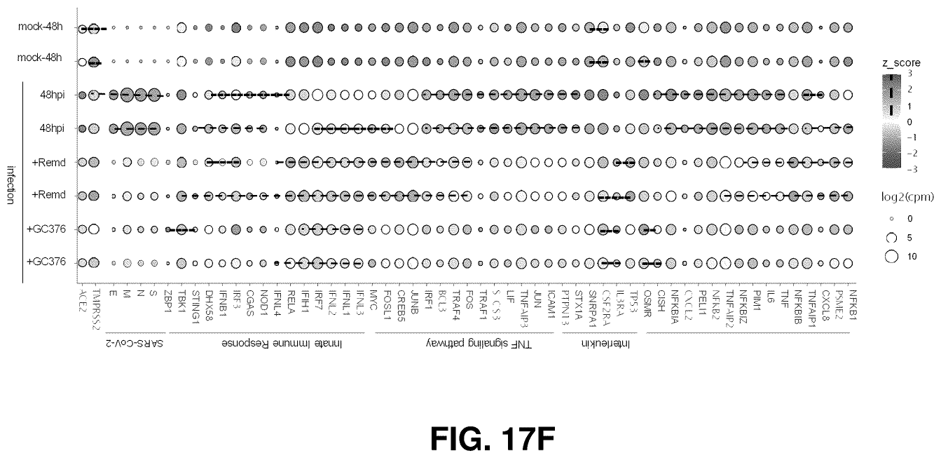

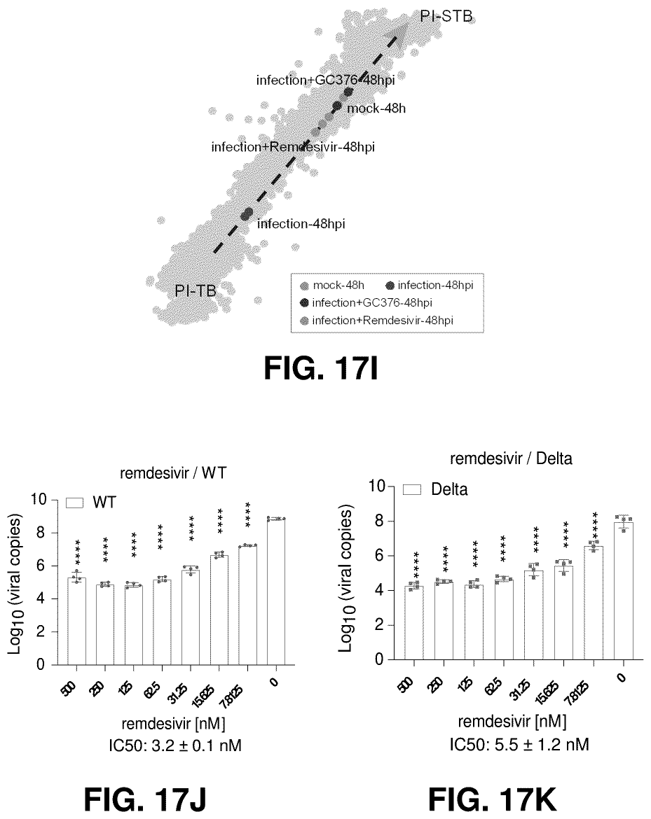

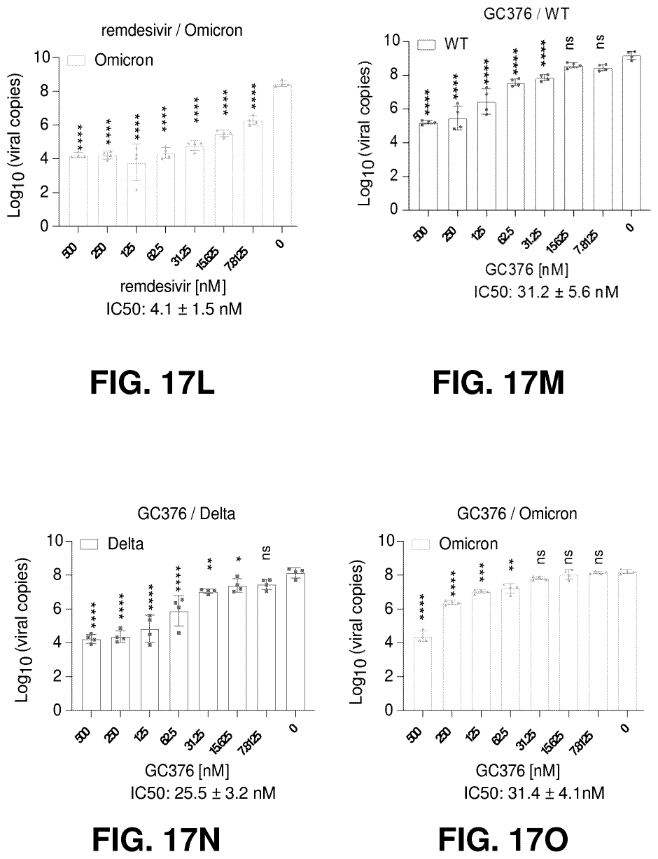

eSTBs permitted robust SARS-CoV-2 infection and thus provide normal and physiologically relevant cells for evaluating antiviral drugs. To this end, the FDA-approved antiviral drug remdesivir (Rubin, D., et al. (2020) N Engl J Med Vol. 383, pages 2598-2600) and a veterinary drug GC376 (Ma, C., et al. (2020) Cell Research Vol. 30, pages 678-692) were tested in the current cell models (FIG. 17A). Remdesivir effectively eliminated SARS-CoV-2 infection in Vero E6 cells at around 5 μM with an IC₅₀ of 0.77 μM (Wang, M., et al. (2020b) Cell Research Vol. 30, pages 269-271). In eSTBs, remarkably, remdesivir demonstrated an IC₅₀ of 3.2±0.1 nM for SARS-CoV-2; and 5.5±1.2 nM for Delta and 4.1±1.5 nM for Omicron variants, respectively (FIGS. 17K and 17L respectively).

GC376 is a repurposed SARS-CoV-2 main protease inhibitor that increases survival of mice with a fatal SARS-CoV-2 infection (Dampalla, C. S., et al. (2021) Proceedings of the National Academy of Sciences Vol. 118, Article e2101555118). It was demonstrated that GC376 suppresses the virus infection in eSTBs with an IC₅₀ of 31.2±5.6 nM against WT, 25.5±3.2 nM against Delta, and 31.4±4.1 nM against Omicron variants, respectively (FIGS. 17N and 17O), which were much lower than in Vero E6 cells (0.70 μM) (Fu, L., et al. (2020) Nature Communications Vol. 11, Article 4417). The effectiveness of these two drugs against SARS-CoV-2 in eSTBs was further confirmed by the substantially reduced viral N antigen expression in immunofluorescence staining.

In naïve-eSTBs, similarly low IC₅₀ of remdesivir (FIGS. 21E-21G) and GC376 (FIGS. 21H-21J) against SARS-CoV-2 and the Delta and Omicron variants. Expression of the DPP4 gene, the host entry receptor of MERS-CoV, another highly pathogenic human coronavirus, is highly correlated with ACE2 and ENPEP in human preimplantation embryos (Qi, F., et al. (2020 Biochem Biophys Res Commun Vol. 526, pages 135-140) and was detected in eSTBs. MERS-CoV infected eSTBs derived from hEPSCs at an efficiency comparable to that in Vero E6 cells (de Wilde, A. H., et al. (2013) J Gen Virol Vol. 94, pages 1749-1760), and both remdesivir (FIG. 17C) and GC376 (FIG. 17E) could effectively block MERS-CoV replication in eSTBs, with an efficiency comparable to that in Vero E6 cells. These results indicate that eSTBs can serve as a good model for antiviral evaluation of SARS-CoV-2, MERS-CoV and probably other coronaviruses.

Next, the global gene expression of the infected and drug-treated cells was examined. Both drug treatments drastically reduced expression of SARS-CoV-2 genes (E, M, N, and S) (FIG. 17F). Neither drug appeared to cause substantial changes of host innate immune response genes such as MDA5, IFNL1-4, IFNB1 and OAS, but proinflammatory cytokine genes such as TNF, IL-6 and IL-8 were all down-regulated in the drug treated cells (FIG. 17F).

Because SARS-CoV-2 infection of eSTBs impaired STB differentiation as presented in FIG. 16A, it was questioned whether remdesivir and GC376 treatment could mitigate this developmental defect. Indeed, hierarchical clustering analysis confirmed a shift of the infected-drug-treated cells towards the mock infection control STBs. After drug treatment, up-regulated and down-regulated genes in the infected cells were substantially reduced when compared to those in the mock infected cells (FIGS. 17G and 17H). Furthermore, machine learning analysis based on global expression decomposition revealed that, along the PI-TB to PI-STB pseudotime trajectory, remdesivir and GC376 treatment of the infected STB cells induced a shift away from PI-TB and toward PI-STB, indicating that antiviral treatment drugs partially rescued eSTB development and differentiation (FIG. 17I).

### Example 8: Direct Derivation of 3D Trophoblast Organoids from EPSC-TSCs for SARS-CoV-2 Infection

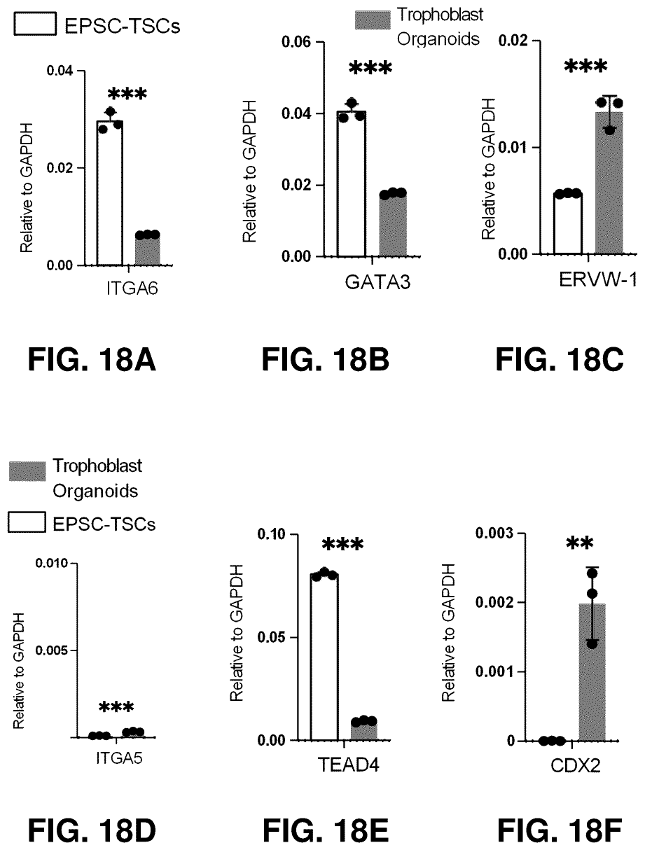

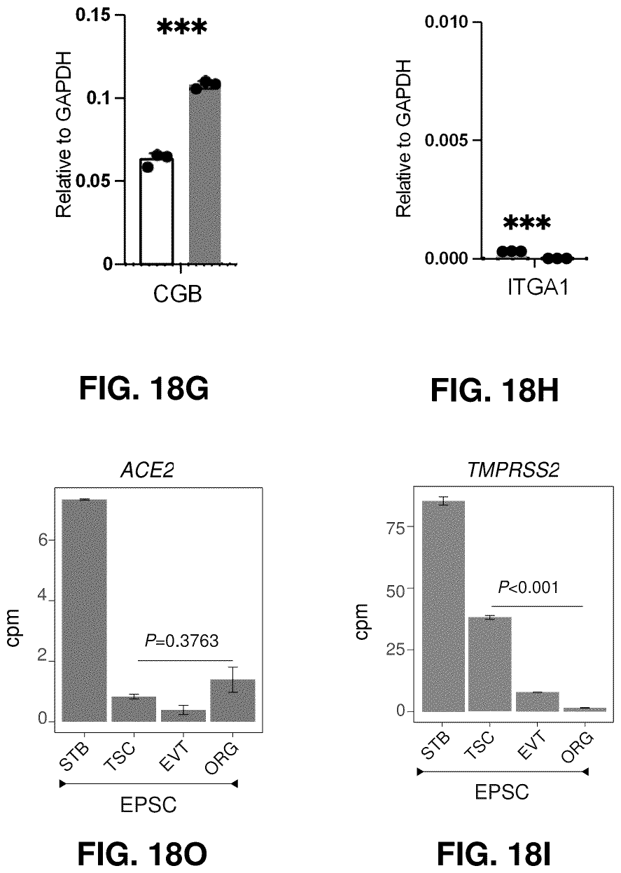

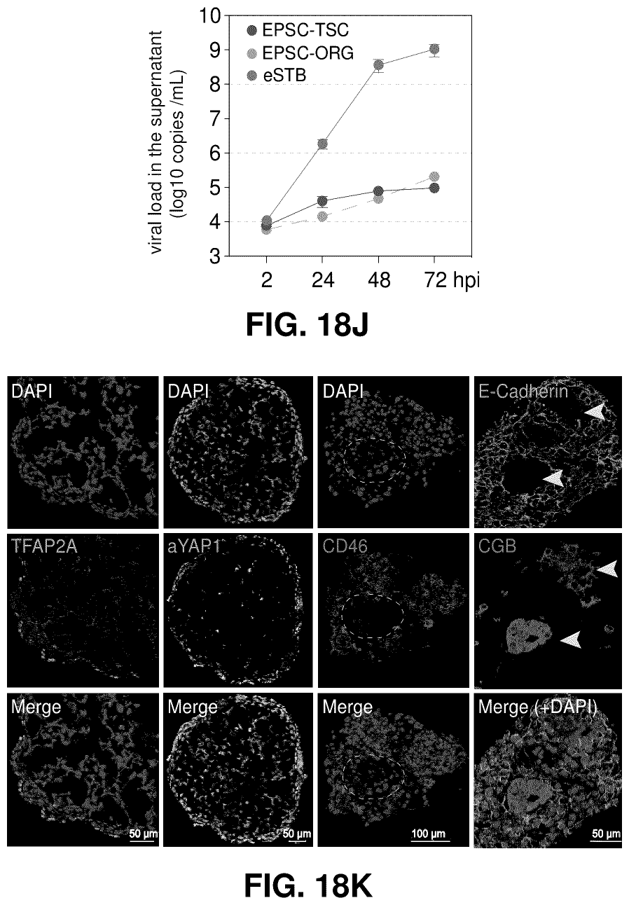

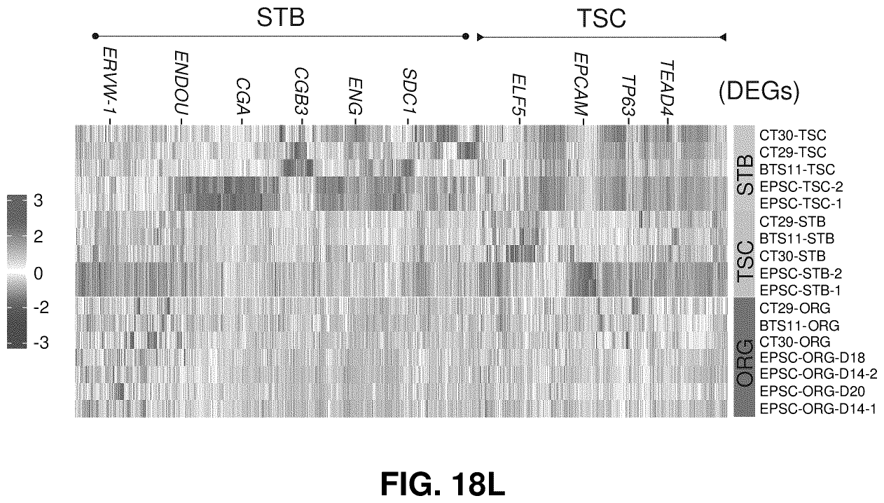

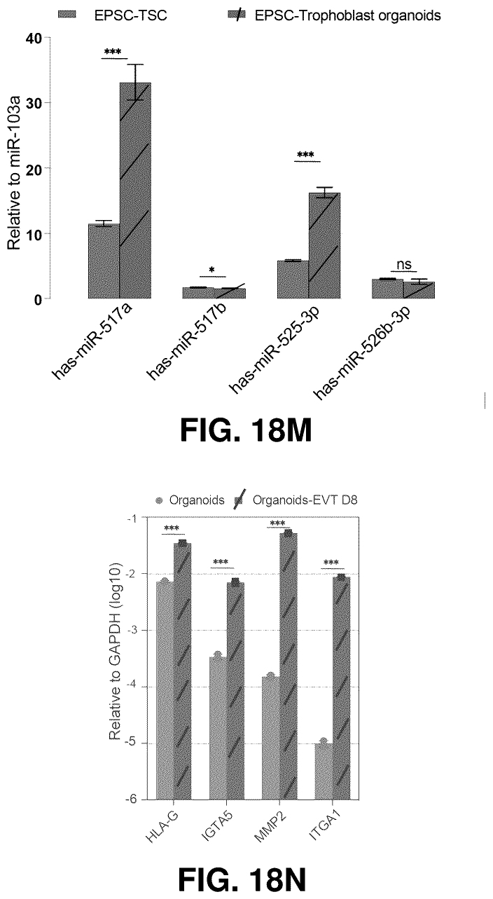

Organoids, which undergo self-renewal and exhibit more physiological relevance to the development and disease pathology in vivo, can serve as powerful platforms for studying COVID-19 in vitro. Various types of human organoids, particularly respiratory cells, have been extensively investigated for SARS-CoV-2 infection (Han, Y., et al. (2022) Nature Methods). Long term and genetically stable trophoblast organoids are derived from first trimester placenta tissues or the blastocyst, which grow as complex structures that closely recapitulate the organization of in vivo placental villi (Haider, S., et al. (2018) Stem Cell Reports Vol. 11, pages 537-551; Turco, M. Y., et al. (2018) Nature Vol. 564, pages 263-267). It was recently reported that 3D or organoid culture of hTSCs more closely resembled the in vivo counterparts than 2D hTSCs (Sheridan, M. A. et al., (2021). Development 148(41): article dev199749). The observation that human trophoblasts were susceptible to SARS-CoV-2 prompted the investigation of whether the 3D trophoblast organoids could be adopted for studying SARS-CoV-2 infection. From EPSC-TSCs, established trophoblast organoids were established under a published culture condition (Sheridan, M. A., et al. (2020) Nature Protocols Vol. 15, pages 3441-3463), where individual hTSCs self-aggregated into small clumps in a few days and eventually matured into 3D organoids in about 20 days. Observed were the morphologies of single cell dissociated EPSC-TSCs (day 0), and cell clumps on day 4 and trophoblast organoids on day 20 (data not shown). The dissociated cells were typically round cells. The organoids were mechanically dissociated, passaged, and maintained for at least 10 passages. These hTSC-derived organoids self-organized into villous-like structures which were reminiscent of those derived from the placenta (Haider, S., et al. (2018) Stem Cell Reports Vol. 11, pages 537-551; Turco, M. Y., et al. (2018) Nature Vol. 564, pages 263-267), where the basement membrane was on the outside in contact with the Matrigel substratum, whereas syncytial masses lined the central cavity. H&E staining of cryosections and paraffin sections of trophoblast organoids were examined. Cells at the organoid peripheral were progenitors (Haider et al., 2018; Turco et al., 2018). Densely packed cell clusters in the outer layer of trophoblast organoids were observed in the cryosections. The multinucleated STBs inside the trophoblast organoid were observed in the paraffin sections. Immunofluorescence staining paraffin sections of SARS-CoV-2 infected trophoblast organoids revealed the expression of GATA3, CD46, E-Cadherin and CGB. Most infected cells were stained positive for CD46 but were low or negative for CGB. White arrows indicate the mononucleated infected cells, whereas yellow arrows point to a large multinucleated STB which expresses high CGB. Multinucleated mature STBs expressing CGB, and ENDON were found at the center of the organoids and most of them did not highly express the stemness transcription factors GATA3, TFAP2A and TFAP2C or the eSTB marker CD46 (FIG. 18K). Trophoblast organoids harbored both stem cells and STBs (Haider, S., et al. (2018) Stem Cell Reports Vol. 11, pages 537-551; Turco, M. Y., et al. (2018) Nature Vol. 564, pages 263-267). They expressed genes of both hTSCs and STBs including GATA3, ITGA6 and TEAD4, and ERVW-1 and CGB, although the levels were lower compared to those in hTSCs or STB cultures, respectively (FIG. 18L). Nevertheless, ELISA detected full-length and properly folded 3-hCG hormone secreted by trophoblast organoids. Consistent with a recent study (Sheridan, M. A. et al., (2021). Development 148(41): article dev199749), trophoblast organoids expressed higher levels of trophoblast-specific miRNAs (has-miR-517a and 525-3p) compared to the 2D TSCs (FIG. 18M). Although trophoblast organoids did not express appreciable levels of EVT genes, they could be induced to robustly generate migrating HLA-G+ and ITGA5+ EVT cells (FIG. 18N), confirming the presence of bi-potential stem cells.

RNA-seq analysis further unraveled the global gene expression similarities between trophoblast organoids derived from EPSC-TSCs (EPSC-ORGs) and from placenta CT-TSCs (CT-TSC-ORGs) (Sheridan, M. A. et al., (2021). Development 148(41): article dev199749). Hierarchical clustering of differentially expressed genes (DEGs) between TSCs and STBs indicates that EPSC-ORGs showed co-expression of markers for both TSCs and STBs, similar to the trophoblast organoids derived from primary villous placenta consisting of villous cytotrophoblasts (vCTBs) and STBs (Haider, S., et al. (2018) Stem Cell Reports Vol. 11, pages 537-551; Turco, M. Y., et al. (2018) Nature Vol. 564, pages 263-267)(FIG. 18L).

To investigate trophoblast organoid SARS-CoV-2 infection, the expression of ACE2 and TMPRSS2 was examined and found that ACE2 was expressed at levels comparable to that in EPSC-TSCs (FIG. 18O) but TMPRSS2 being much lower in EPSC-ORGs (FIG. 18I), resembling that in the placenta (Ashary, N. et al. (2020). Frontiers in Cell and Developmental Biology Vol. 8, Article 783; Chen, W. et al. (2020) Engineering (Beijing) Vol. 6(10), pages 1162-1169; Pique-Regi, R., et al. (2020) Elife Vol. 9). EPSC-TSC-derived 20-day-old trophoblast organoids were next infected with SARS-CoV-2 at an MOI of 10 inspired by previous studies for SARS-CoV-2 infection in organoids (Zhang, B.-Z., et al. (2020) Cell Research Vol. 30, pages 928-931). SARS-CoV-2 RNA copy number in the supernatants from infected trophoblast organoids was detected at levels comparable to EPSC-TSC infection (FIG. 18J). Immunofluorescence staining revealed that a small number of cells located along the periphery of the trophoblast organoids were positive for SARS-CoV-2 N, which varied substantially among organoids possibly due to organoid's cellular heterogeneity. The infected cells tended to co-express ACE2, CD46 and E-cadherin but not CGB, although the co-expression was much more clearly demonstrated in 2D cultured eSTBs. The low SARS-CoV-2 infection in the trophoblast organoids in vitro supports the clinical observations that opportunistic SARS-CoV-2 infection of the human placenta can occur but are uncommon (Male, V. (2022) Nature Reviews Immunology; Roberts, et al. (2021) American journal of obstetrics and gynecology Vol. 225(593), pages e591-593.e599).

### Example 9: ACE2 is Essential for SARS-CoV-2 Infection in Trophoblasts

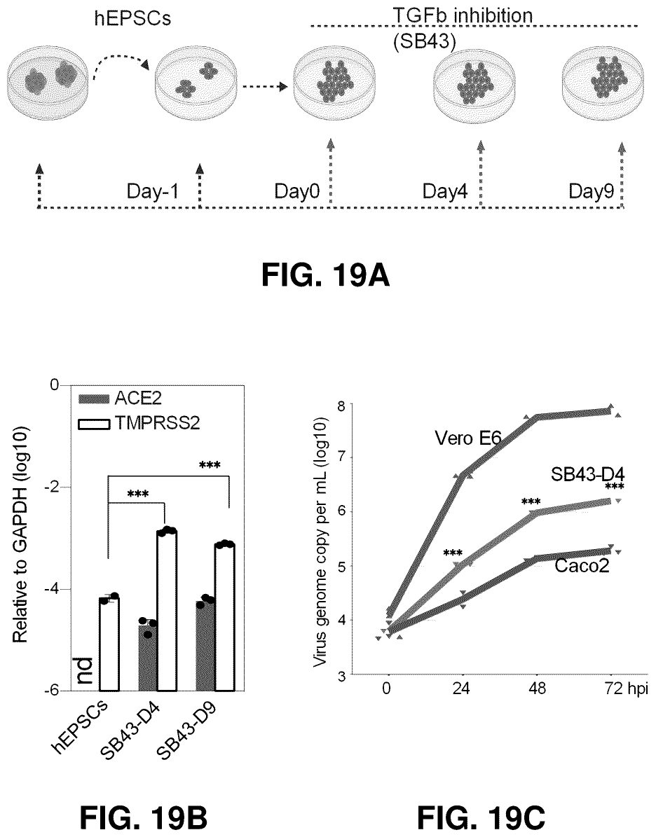

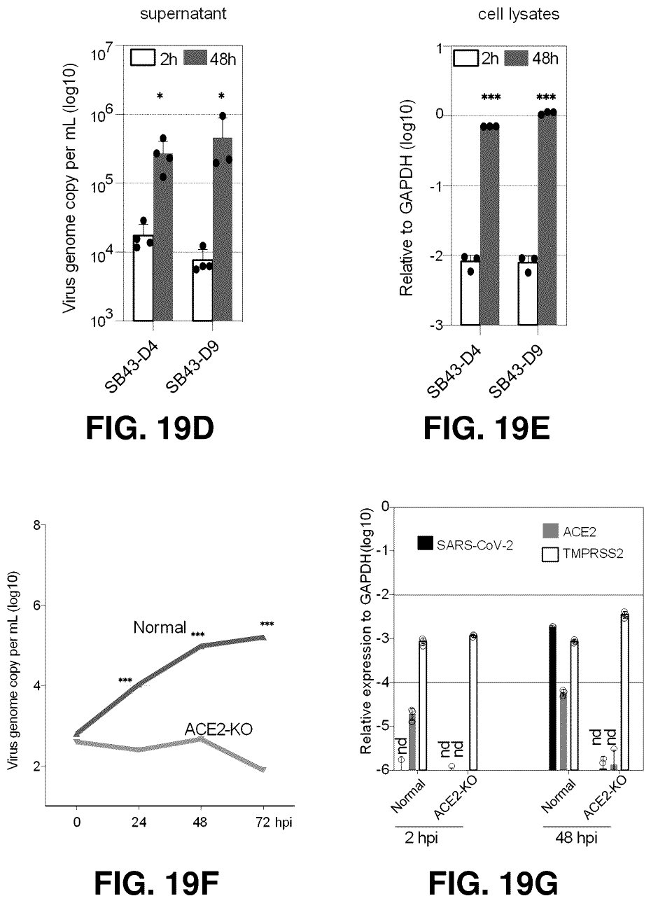

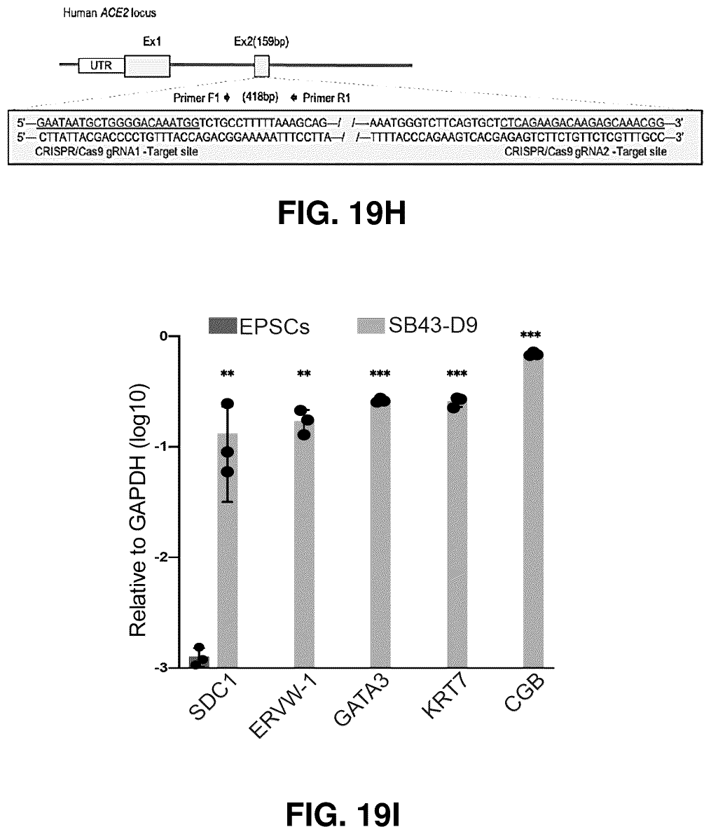

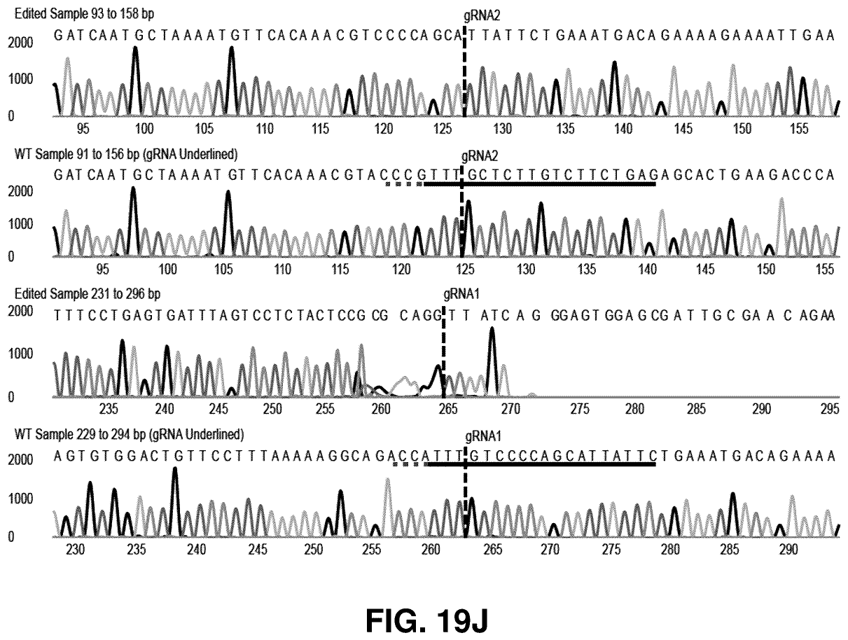

In the peri-implantation embryos, PI-STBs co-expressed high levels of ACE2 and TMPRSS2. All the SARS-CoV-2 infected trophoblasts regardless of cell origin expressed ACE2. To genetically validate the role of ACE2 in SARS-CoV-2 infection in human trophoblasts, homozygous deletions in ACE2 coding exon 2 in hEPSCs were made using the CRISPR/Cas9 (FIGS. 19H and 19J, Table 5 and Table 6). The ACE2 knockout hEPSCs (ACE2-KO) had normal morphology and expressed high levels of key pluripotency genes (OCT4 and NANOG) and markers (SSEA34 and TRA-1-60) but low levels of lineage genes (CDX2, ELF5, SOX17, GATA6, SOX1) (FIG. 19I), comparable to the normal parental hEPSCs.

TABLE 5
Sanger sequencing of the mutant PCR fragment reveals a 148 bp deletion
between the two CRISPR gRNAs in exon 2 as expected.
Sanger sequencing

| | |
|---|---|
| wildtype | 5′-GTTCACAAACGTACCCGTTTGCTCTTG--//-GGCAGACCATTCCCCAGCATTATTCTGAAAT-3′ |
| Knock-out | 5′-GTTCAAACGT------------(148bp)--------------CCCCAGCATTATTOTGAAAT-3' |

TABLE 6
Genome editing efficiency at the ACE2 locus in hEPSCs.
Five out 24 genotyped colonies were the homozygous mutant ones.
summary of ACE2 Knock-out in EPSCs

| | no. of picked | no. of | no. of | no. of |
|---|---|---|---|---|
| cell line | single clones | genotyped | heterozygote | homozygote |
| M1 EPSCs | 24 | 24 | 4 | 5 |

Normal and ACE2-KO hEPSCs were induced to trophoblasts using a simple and efficient protocol by the TGF-β inhibitor SB431542 (Gao, X. et al. (2019) Nature Cell Biology 21(6), pages 687-699) (FIG. 19A). Normal hEPSCs did not express ACE2 (FIG. 19B) and were not infected by SARS-CoV-2 as described above. The differentiated cells expressed ACE2, TMPRSS2, and typical STB markers such as SDC1, ERVW-1 (SYNCYTIN-1), GATA3, and KRT7, and CGB and CD46 (FIG. 19B and FIG. 19I). In contrast, no ACE2 protein was detected in cells differentiated from the ACE2-KO hEPSCs. Western blotting confirmed the loss of ACE2 protein in the SB43-treated hEPSCs on day 9 (SB43-D9) of differentiation.

The cells differentiated from normal and ACE2-KO hEPSCs were subsequently subjected to SARS-CoV-2 infection. Cells of day-4 and day-9 differentiation were selected for infection as they expressed ACE2 and TMPRSS2 (FIG. 19B). Infection of these cells produced substantial amounts of viral genome in the supernatant and cell lysates (FIG. 19G). The infected cells, detected as SARS-CoV-2 nucleocapsid (N) protein-positive, all expressed ACE2 and trophoblast factor and markers TFAP2C, SSEA4 and KRT7, and most were mononucleated. Loss of ACE2 abolished the infection as indicated by the drastic decrease of viral genome in the supernatants (FIG. 19F) and in cell lysates (FIG. 19G). In immunofluorescence staining, no cells were stained positive for either SARS-CoV-2 N protein or ACE2 in the cells differentiated from ACE2-KO EPSCs (Therefore, in hEPSC-derived trophoblasts, ACE2 is essential for SARS-CoV-2 infection.

### SUMMARY

It has been challenging both technically and ethically to directly study trophoblast SARS-CoV-2 infection in pregnancy in general and in early pregnancy in particular. COVID-19 cases in many countries are dropping, however, the emergence of SARS-CoV-2 variants of concern with progressively increased transmissibility and jeopardizing the protective antiviral immunity induced following infection or vaccination still posts a threat to global public health (DeGrace, M. M., et al. (2022) Nature). Reported in the present study is a stem cell-based system to study human trophoblast's susceptibility to SARS-CoV-2, and its variants and MERS-CoV. Specifically, human trophoblast stem cells (hTSCs) originated from EPSCs, naïve stem cells and the in vivo cells (the blastocyst) were used to generate STBs and EVTs for SARS-CoV-2 infection.

An important issue about hTSCs from in vitro stem cells is that they may possess AME gene signature (Gao, X. et al. (2019) Nature Cell Biology 21(6), pages 687-699; Guo, G., et al. (2021) Cell Stem Cell Vol. 28, pages 1040-1056 e1046). The transcriptomic identity of hTSCs originated from stem cells were examined by comparing them to those of in vivo origins. All the hTSCs expressed low or no of those genes that are thought to be enriched in putative AME cells. The hTSCs of EPSCs were examined, naïve stem cells and the blastocyst and found that they were highly similar in differentiation capacity and in susceptibility to SARS-CoV-2 and its variants.

hTSCs are thought to represent in vivo post-implantation cells (Castel, G. et al. (2020). Cell Reports Vol. 33(8), Article 108419). SARS-CoV-2 infected 0.5-4% hTSCs that were ACE2+ and co-expressed ENPEP, a trophectoderm marker gene. The presence of ACE2+ cells in the current hTSC cultures warrants further investigation for their molecular properties and developmental potential and for the in vivo counterparts.

hTSCs could be readily induced to differentiate to both STBs and EVTs. Remarkably Day-2 STB or eSTBs expressed high levels of both ACE2 and TMPRSS2 and potently supported the replication of SARS-CoV-2 and the Delta and Omicron variants and of MERS-CoV. The infection was effectively suppressed, and the development delay caused by the infection was partially rescued by the two known antiviral drugs, remdesivir and GC376. hTSCs and eSTBs have enriched transcriptomic features of peri-implantation trophoblasts. Their susceptibility to coronavirus and the resultant developmental defects raises the possibility that early pregnancy is at risk of COVID-19 infection which may not easily be noticed if it occurs at peri-implantation stages, which warrants future clinical investigation. SARS-CoV-1, which caused SARS pandemic in 2003, also uses ACE2 for cell entry. SARS patients were reported to have first trimester miscarriage (Wong, S. F., et al. (2004) Am J Obstet Gynecol Vol. 191, pages 292-297).

The syncytialized trophoblasts generated from human ESCs (primed) by BAP treatment (BMP4, plus a TGFβ inhibitor A83-01 and a FGF2 signaling inhibitor PD173074) were found to co-express ACE2 and TMPRSS2 and supported replicative and persistent infection by SARS-CoV-2 (Zhou, J., et al. (2021) The Journal of Infectious Diseases Vol. 224, pages S660-S669). These results are different from the data of the present study that the early STBs derived from hTSCs had high ACE2 and were highly susceptible to SARS-CoV-2 infection whereas the syncytialized or more mature STBs didn't and were much less susceptible. The discrepancy could be due to either technical reasons as eSTBs are expected to be only transiently present in hTSC differentiation, or to the difference between BAP cells (primitive trophoblasts) and hTSCs/eSTBs. Indeed, the discovery of eSTBs in the present study was made possible by the stem cell-based in vitro system since eSTB-like cells also likely only transiently exist in the placenta trophoblast development before they differentiate to the non-proliferative multinucleated STBs. Neither trophoblastic cell lines nor primary trophoblasts might be representative of these eSTBs. Importantly, the findings of the present study are in line with the clinical reports that opportunistic SARS-CoV-2 infection during pregnancy can occur but is an infrequent event. Nevertheless, in both eSTBs and the primitive trophoblasts, co-expression of ACE2 and TMPRSS2 in these in vitro derived cells again indicate a possible risk in early pregnancy. A recent study indeed highlights that one health benefit of vaccination are statistically better pregnancy outcomes (Stock, S. J., et al. (2022) Nat Med).

Several human cell lines have been used in coronavirus research. However, they suffer from genetic and/or innate immune defects and have some of the long-standing technical challenges such as cell transfection and genetic manipulation. Vero E6 cells originated from African green monkey are commonly used for isolation and propagation of SARS-CoV-2 (Zhou, P., et al. (2020) Nature Vol. 579, pages 270-273) and have been approved for use in vaccine manufacturing. They have genetic defects including large genomic deletions encompassing the interferon gene clusters (IFN-α and -b) and the CDKN2A/B loci and are thus deficient in IFN response (Osada, N., et al. (2014) DNA Res Vol. 21, pages 673-683), which have prevented its application in the study of antiviral response. Furthermore, the lack of TMPRSS2 in Vero E6 cells results in the selection of SARS-CoV-2 variants that have lost the polybasic Furin cleavage site at the S1-S2 junction (Sasaki, M., et al. (2021) PloS Pathog Vol. 17, Article e1009233). On the other hand, the current dominant SARS-CoV-2 variant Omicron, which relies more on Cathepsins and other endosomal proteases, replicates well in Vero E6 cells and the upper airway that are more abundant in ACE2 and cathepsins, but not in Calu3 and Caco2 cells or in lung cells (Hui, K. P. Y., et al. (2022) Nature Vol. 603, pages 715-720; Shuai, H., et al. (2022). Nature). eSTBs express high levels of ACE2 and TMPRSS2 that SARS-CoV-2 uses for entry into cells, and support highly productive SARS-CoV-2 propagation. eSTBs also express the Cathepsins important for the virus entry and permitted robust Omicron replication.

In contrast to Vero E6 cells, stem cell-generated trophoblasts are normal human cells with an intact innate immune system but without major known genetic or epigenetic defects. SARS-CoV-2 infection causes massive cell death in Vero E6 cells but not in eSTBs. The lack of cell death in the infected eSTBs is different from recent reports that the primitive human trophoblasts generated from human ESCs were susceptible to an African lineage strain Zika virus, which lysed the trophoblasts (Sheridan, M. A., et al. (2018) PloS One Vol. 13, Article e0200086; Sheridan, M. A., et al. (2017) Proceedings of the National Academy of Sciences of the United States of America Vol. 114, pages E1587-e1596).

Although eSTBs are highly susceptible to SARS-CoV-2 infection in 2D culture, COVID-19 placenta infection is uncommon. It was reported that the placenta, in late pregnancy, expresses little ACE2 and TMPRSS2 (Pique-Regi, R., et al. (2020) Elife Vol. 9). Indeed, one report claimed that term placenta trophoblasts were not susceptible to SARS-CoV-2 infection (Colson, A., et al. (2021) Am J Pathol Vol. 191, pages 1610-1623). To better understand these in vivo data, hTSC-derived trophoblast organoids were generated and tested to model the 3D placenta infection. The trophoblast organoids expressed low levels of ACE2 and TMPRSS2 and were much less susceptible to the virus infection, reminiscent of the placenta.

Human EPSCs and naïve stem cells permit efficient genome editing and have the developmental potential to generate all embryonic and extraembryonic cell lineages (Gao, X. et al. (2019) Nature Cell Biology 21(6), pages 687-699; Guo, G. et al. (2021). Cell Stem Cell 28(6), pages 1040-1056. E6). Besides trophoblasts, these stem cells can be explored to derive diverse cell types and to functionally assess their susceptibility to SARS-CoV-2 and its variants, including airway epithelial, gastrointestinal epithelial and neuronal cells. These qualities grant them potentials in overcoming some of the long-standing technical challenges such as cell transfection and genetic manipulation of various target cells. Cells generated from these stem cells, in particular eSTBs, may thus represent a new normal human cell source for investigating interactions between the hosts and the viruses of the current and upcoming pandemics, for performing genetic screens to identify biological determinants of the infection and for antiviral evaluation and discovery.

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a component, such as a cell, is disclosed and discussed and a number of modifications that can be made to a number of components including the cell are discussed, each and every combination and permutation of cell and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a,” “an,” and “the” include plural reference unless the context clearly dictates otherwise. Thus, for example, reference to “a cell” includes a plurality of such cells, reference to “the cell” is a reference to one or more cells and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different types of components does not indicate that the listed types of components are obvious one to the other, nor is it an admission of equivalence or obviousness.

Every cell disclosed herein is intended to be and should be considered to be specifically disclosed herein. Further, every subgroup of cells that can be identified within this disclosure is intended to be and should be considered to be specifically disclosed herein. As a result, it is specifically contemplated that any cell, or subgroup of cells can be either specifically included for or excluded from use or included in or excluded from a list of cells.

Those skilled in the art will recognize, or be able to ascertain, using no more than routine experimentation, many equivalents to the specific forms of the method and compositions described herein. Such equivalents are intended to be encompassed by the following claims.

We claim:

1. A method comprising incubating early syncytiotrophoblasts (eSTBs) in coronavirus medium comprising coronavirus particles, whereby the coronavirus particles infect and replicate in the eSTBs.

2. The method of claim 1, wherein the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles or variant particles thereof.

3. The method of claim 1, wherein the coronavirus particles are non-human coronavirus particles selected from the group comprising canine enteric coronavirus (CECoV) particles, feline coronavirus (FCoV) particles, porcine respiratory coronavirus (PRCV) particles, porcine epidemic diarrhea virus (PEDV) particles, transmissible gastroenteritis virus (TGEV) particles, canine respiratory coronavirus (CRCoV) particles, murine coronavirus (M-CoV) particles, porcine hemagglutinating encephalomyelitis virus (PHEV) particles, porcine enteric coronavirus (PEC) particles, swine acute diarrhea syndrome coronavirus (SADS-CoV), porcine delta coronavirus (PDCoV), hedgehog coronavirus 1 particles, bovine coronavirus (B-CoV) particles, equine coronavirus (E-CoV) particles, tylonycteris bat coronavirus HKU4 (Bat-CoV HKU4) particles, pipistrellus bat coronavirus HKU5 (Bat-CoV HKU5) particles, rousettus bat coronavirus HKU9 (Bat-CoV HKU9) particles, Avian Infectious Bronchitis (AIBV) particles, Beluga Whale CoV SW1 coronavirus particles and variant particles thereof.

4. The method of claim 1, wherein the coronavirus particles are SARS-CoV-2 particles selected from the group comprising SARS-CoV-2 alpha variant particles, SARS-CoV-2 beta variant particles, SARS-CoV-2 gamma variant particles, SARS-CoV-2 delta variant particles, SARS-CoV-2 epsilon variant particles, SARS-CoV-2 eta variant particles, SARS-CoV-2 iota variant particles, SARS-CoV-2 kappa variant particles, SARS-CoV-2 mu variant particles, SARS-CoV-2 omicron variant particles, SARS-CoV-2 zeta variant particles, SARS-CoV-2 1.617.3 variant particles and SARS-CoV-2 lambda variant particles.

5. The method of claim 1, wherein the eSTBs are primarily mononucleated cells and are not multi-nucleated or mature cells.

6. The method of claim 1, wherein the derived eSTBs are isolated by selecting cells expressing eSTB-like morphology, cells expressing an eSTB-like molecular signature, or cells expressing both eSTB-like morphology and an eSTB-like molecular signature,
wherein the eSTB-like molecular signature comprises the increased early STB markers CD46 and/or SSEA4; one or more increased STB markers, wherein the increased STB markers are GCM1, β chorionic gonadotrophin 3 gene (CGB3), CGB5, CD46, ENG, and/or CSH2, decreased mature STB markers such as trophoblast progenitor transcription factor TP63, and/or properly folded or secreted β-hCG hormone.

7. The method of claim 6, further comprising assessing a portion of the isolated eSTBs for coronavirus susceptible markers,
wherein the assessment of the portion of the eSTBs for coronavirus susceptible markers is done via a virus replication kinetic assay selected from the group comprising RT-qPCR, plaque assays, Trans-well invasion assays, and/or RNA sequencing, and
wherein the coronavirus susceptible markers are increased ACE2 and/or increased TMPRSS2.

8. The method of claim 1, further comprising quantifying the coronavirus load in the coronavirus medium,
wherein quantification of the coronavirus load is done via a plaque assay, RT-qPCR, and/or RNA sequencing, and
wherein the coronavirus particles are SARS-CoV-2 particles.

9. The method of claim 1, further comprising assessing the derived eSTBs for cytopathic effects,
wherein the eSTBs are incubated with the coronavirus medium comprising the coronavirus particles at about 37° C. for about 2 hours, and
wherein the coronavirus particles are diluted in the coronavirus medium, wherein the coronavirus medium comprises basal medium, wherein basal medium is DMEM/F-12 or DMEM.

10. The method of claim 1, wherein the coronavirus particles are isolated from a sample comprising the coronavirus particles,
wherein the sample comprising the coronavirus particles is coronavirus-infected VeroE6 cells or a directly obtained patient sample.

11. The method of claim 1, wherein the eSTBs are incubated in the coronavirus medium for about 1 day to 6 days.

12. The method of claim 1, wherein the eSTBs are derived from trophoblast stem cells (TSCs),
wherein the TSCs are human TSCs, bovine TSCs, ovine TSCs, porcine TSCs, canine TSCs, feline TSCs, equine TSCs, or non-human primate TSCs,
wherein the eSTBs are derived by culturing the TSCs in STB medium for about 1 to about 6 days, and
wherein the number of TSCs used to derive the eSTBs is about 0.5×10⁵/cm² to about 2.0×10⁵/cm², preferably about 1.0×10⁵/cm²TSCs.

13. The method of claim 12, wherein the STB medium comprises basal medium supplemented with one or more of a reducing agent, BSA, an antibiotic, a ROCK inhibitor, a cAMP inhibitor, KSR medium, and one or more differentiation agents,
wherein the basal medium is DMEM/F-12 or DMEM,
wherein the antibiotic is Penicillin-Streptomycin-Glutamine at about 0.5 weight percent,
wherein the reducing agent is β-mercaptoethanol in a concentration of about 50 μM,
wherein the ROCK inhibitor is Y-27632 in a concentration of about 2.5 μM,
wherein the cAMP inhibitor is forskolin in a concentration of about 2 μM, and
wherein the differentiation agent is ITS-X at about 1%.

14. The method of claim 12, wherein the TSCs are derived from reprogrammed somatic cells such as from fibroblasts or blood cells or from expanded potential stem cells (EPSCs), primed stem cells, naïve stem cells, embryonic stem cells, induced pluripotent stem cells, other pluripotent stem cells, peri-implantation embryos, placental tissues or genetically altered derivatives thereof.

15. The method of claim 14, wherein the derived TSCs are identified by screening the TSC colonies for expression of TSC-like morphology and/or a TSC-typical molecular signature,
wherein the TSC-typical molecular signature is selected from the group comprising increased TSC factors, increased trophoblast-specific miRNAs, decreased HLA class I molecules, and decreased AME genes,
wherein the increased TSC factors are selected from the group comprising TFAP2C, TP63, CK18, GATA3, ELF5, TEAD4, and/or KRT7,
wherein the increased trophoblast-specific miRNAs are selected from the group comprising has-miR-517c-3p, 517-5p, 525-3p, and/or 526b-3p,
wherein the decreased HLA class I molecules are selected from the group comprising HLA-A and HLA-B, and
wherein the decreased AME genes are selected from the group comprising CDX2, MUC16, GABRP, ITGB6, and/or VTCN1.

16. The method of claim 14, further comprising re-plating the TSCs and passaging the TSCs one or more times.

17. The method of claim 15, wherein when EPSCs are used to derive the TSCs, the TSCs are derived by:
(i) culturing dissociated EPSCs for about 24 hours in a first culture medium comprising one or more of knock-out serum replacement (KSR) medium, growth factors, and a ROCK inhibitor,
wherein the number of EPSCs used in the first culture medium is about 0.5×10⁵ cells to about 2.0×10⁵ cells per well of a 6-well cell culture dish, preferably about 1×10⁵ cells per well,
wherein the ROCK inhibitor is Y27632 or thiazovivin in a concentration of at least about 2 μM to about 10 μM;
(ii) culturing the EPSCs from step (i) in a second culture medium comprising a TGF-β inhibitor and KSR medium,
wherein the TGF-β inhibitor is SB431542 or A83-01 in a concentration of about 2 μM to about 10 μM; and
(iii) culturing the EPSCs from step (ii) in a third culture medium comprising human trophoblast-induced stem cell medium, thereby producing TSC colonies,
wherein the number of hEPSCs used in the third culture medium is about 2000 cells per well to about 10,000 cells per well.

18. One or more coronavirus infected eSTBs produced by the method of claim 1.

19. A method of screening an agent for an effect on coronavirus infected cells, the method comprising contacting the coronavirus infected eSTBs of claim 1 with the agent and determining the effect of the agent on survival, proliferation, differentiation, virus production, or morphologic, genetic, or functional parameters of the eSTBs.

20. The method of claim 19, wherein the effect of the agent is indicative of the agent being safe for treatment of a pregnant subject infected with coronavirus, a fetus infected with coronavirus, or both.

21. The method of claim 19, wherein the agent is an antiviral agent, wherein the effect of the agent is indicative of the agent being safe for treatment of viral infections.

22. The method of claim 19, wherein the coronavirus particles are Human Coronavirus 229E (HCoV-229E) particles, Human Coronavirus OC43 (HCoV-OC43) particles, Human Coronavirus NL63 (HCoV-NL63) particles, Human Coronavirus HKU1 (HCoV-HKU1) particles, Middle East Respiratory Syndrome Coronavirus (MERS-CoV) particles, Severe Acute Respiratory Syndrome Coronavirus 1 (SARS-CoV-1) particles, SARS-CoV-2 particles or variant particles thereof.

23. The method of claim 19, wherein the coronavirus particles are SARS-CoV-2 particles selected from the group comprising SARS-CoV-2 alpha variant particles, SARS-CoV-2 beta variant particles, SARS-CoV-2 gamma variant particles, SARS-CoV-2 delta variant particles, SARS-CoV-2 epsilon variant particles, SARS-CoV-2 eta variant particles, SARS-CoV-2 iota variant particles, SARS-CoV-2 kappa variant particles, SARS-CoV-2 mu variant particles, SARS-CoV-2 omicron variant particles, SARS-CoV-2 zeta variant particles, SARS-CoV-2 1.617.3 variant particles and/or SARS-CoV-2 lambda variant particles.

24. The method of claim 19, wherein the coronavirus particles are non-human coronavirus particles selected from the group comprising canine enteric coronavirus (CECoV) particles, feline coronavirus (FCoV) particles, porcine respiratory coronavirus (PRCV) particles, porcine epidemic diarrhea virus (PEDV) particles, transmissible gastroenteritis virus (TGEV) particles, canine respiratory coronavirus (CRCoV) particles, murine coronavirus (M-CoV) particles, porcine hemagglutinating encephalomyelitis virus (PHEV) particles, porcine enteric coronavirus (PEC) particles, swine acute diarrhea syndrome coronavirus (SADS-CoV), porcine delta coronavirus (PDCoV), hedgehog coronavirus 1 particles, bovine coronavirus (B-CoV) particles, equine coronavirus (E-CoV) particles, tylonycteris bat coronavirus HKU4 (Bat-CoV HKU4) particles, pipistrellus bat coronavirus HKU5 (Bat-CoV HKU5) particles, rousettus bat coronavirus HKU9 (Bat-CoV HKU9) particles, Avian Infectious Bronchitis (AIBV) particles, Beluga Whale CoV SW1 coronavirus particles and variant particles thereof.

25. A method for screening an agent for an effect on early syncytiotrophoblasts (eSTBs), the method comprising contacting the eSTBs with the agent and determining the effect of the agent on survival, proliferation, differentiation, or morphologic, genetic, or functional parameters of the eSTBs.

26. A kit for culturing the coronavirus particles in early syncytiotrophoblasts (eSTBs), the kit comprising a combination of two or more of:
(i) the fourth culture medium for deriving the EPSCs described in A(ii) comprising one or more of a RAS-ERK inhibitor, a SRC kinase inhibitor, a GSK3 inhibitor, and/or a WNT inhibitor;
(ii) the EPSC maintenance medium comprising one or more of a RAS-ERK inhibitor, a SRC kinase inhibitor, a GSK3 inhibitor and/or a WNT inhibitor;
(iii) the dissociating reagent for producing single EPSCs comprising a trypsin replacement agent;
(iv) the trophoblast stem cell medium comprising basal medium supplemented with one or more of a reducing agent, fetal bovine serum (FBS), an antibiotic, Bovine Serum Albumin (BSA), Epidermal Growth Factor (EGF), Glycogen synthase kinase 3 (GSK-3) inhibitor, an ALK-5 inhibitor, a ROCK inhibitor, a TGF-β inhibitor, and/or an HDAC inhibitor, wherein basal medium is DMEM/F-12 or DMEM;
(v) STB medium comprising basal medium supplemented with one or more of a reducing agent, BSA, an antibiotic, a ROCK inhibitor, a cAMP inhibitor, KSR medium, and/or one or more differentiation agents; and
(vi) coronavirus medium comprising basal medium, wherein basal medium is DMEM/F-12 or DMEM.
